(19) United States
(12) Patent Application Publication
(10) Pub. No.: US 2003/0232875 A1
(43) Pub. Date: Dec. 18, 2003

(54) Bicyclic pyrrolyl amides as glucogen phosphorylase inhibitors

(76) Inventors: Julie B Bartlett, Stockport (GB); Sue Freeman, Wilmslow (GB); Peter Kenny, Congleton (GB); Andrew Morley, Macclesfield (GB); Paul Whittamore, Congleton (GB)

Correspondence Address: ROPES & GRAY LLP, ONE INTERNATIONAL PLACE, BOSTON, MA, 02110-2624, US

(21) Appl. No.: 10/344,506

(22) PCT Filed: Aug. 31, 2001
(86) PCT No.: PCT/SE01/01880

(30) Foreign Application Priority Data
Sep. 6, 2000 (GB) 0021831.3

(51) Int. Cl. C07D 487/02; C07D 491/02; A61K 31/407

(52) U.S. Cl. 514/412; 548/453

(57) ABSTRACT

Heterocyclic amide derivatives, of formula (I): wherein —X-Y-Z- is selected from —S—CR⁴═CR⁵—, —CR⁴═CR⁵—S—, —O—CR⁴═CR⁵—, —CR⁴═CR⁵—O—, —N═CR⁴—S—, —S—CR⁴═N—, —NR⁶—CR⁴═CR⁵— and —CR⁴═CR⁵—NR⁶—; or a pharmaceutically acceptable salt or an in vivo hydrolysable ester thereof; (with provisos); possess glycogen phosphorylase inhibitory activity and accordingly have value in the treatment of disease states associated with increased glycogen phosphorylase activity. Processes for the manufacture of said heterocyclic amide derivatives and pharmaceutical compositions containing them are described.

The present invention relates to heterocyclic amide derivatives, pharmaceutically acceptable salts and in vivo hydrolysable esters thereof. These heterocyclic amides possess glycogen phosphorylase inhibitory activity and accordingly have value in the treatment of disease states associated with increased glycogen phosphorylase activity and thus are potentially useful in methods for the treatment of a warm-blooded animal such as man. The invention also relates to processes for the manufacture of said heterocyclic amide derivatives, to pharmaceutical compositions containing them and to their use in the manufacture of medicaments to inhibit glycogen phosphorylase activity in a warm-blooded animal such as man.

The liver is the major organ regulating glycaemia in the post-absorptive state. Additionally, although having a smaller role in the contribution to post-prandial blood glucose levels, the response of the liver to exogenous sources of plasma glucose is key to an ability to maintain euglycaemia. An increased hepatic glucose output (HGO) is considered to play an important role in maintaining the elevated fasting plasma glucose (FPG) levels seen in type 2 diabetics; particularly those with a FPG>140 mg/dl (7.8 mM). (Weyer et al, (1999), J Clin Invest 104: 787-794; Clore & Blackgard (1994), Diabetes 43: 256-262; De Fronzo, R. A., et al, (1992) Diabetes Care 15; 318-355; Reaven, G. M. (1995) Diabetologia 38; 3-13).

Since current oral, anti-diabetic therapies fail to bring FPG levels to within the normal, non-diabetic range and since raised FPG (and glycHbA1c) levels are risk factors for both macro- (Charles, M. A. et al (1996) Lancet 348, 1657-1658; Coutinho, M. et al (1999) Diabetes Care 22; 233-240; Shaw, J. E. et al (2000) Diabetes Care 23, 34-39) and micro-vascular disease (DCCT Research Group (1993) New. Eng. J. Med. 329; 977-986); the reduction and normalisation of elevated FPG levels remains a treatment goal in type 2 diabetes.

It has been estimated that, after an overnight fast, 74% of HGO is derived from glycogenolysis with the remainder derived from gluconeogenic precursors (Hellerstein et al (1997) Am J Physiol, 272: El 63). Glycogen phosphorylase is a key enzyme in the generation by glycogenolysis of glucose-1-phosphate, and hence glucose in liver and also in other tissues such as muscle and neuronal tissue.

Liver glycogen phosphorylase activity is elevated in diabetic animal models including the db/db mouse and the fa/fa rat (Aiston S et al (2000). Diabetalogia 43, 589-597).

Inhibition of hepatic glycogen phosphorylase with chloroindole inhibitors (CP91149 and CP320626) has been shown to reduce both glucagon stimulated glycogenolysis and glucose output in hepatocytes (Hoover et al (1998) J Med Chem 41, 2934-8; Martin et al (1998) PNAS 95, 1776-81, WO 96/39384 and WO 96/39385). Additionally, plasma glucose concentration is reduced, in a dose related manner, in db/db and ob/ob mice following treatment with these compounds.

Studies in conscious dogs with glucagon challenge in the absence and presence of another glycogen phosphorylase inhibitor, Bay K 3401, also show the potential utility of such agents where there is elevated circulating levels of glucagon, as in both Type 1 and Type 2 diabetes. In the presence of Bay R 3401, hepatic glucose output and arterial plasma glucose levels following a glucagon challenge were reduced significantly (Shiota et al, (1997), Am J Physiol, 273: E868).

ES 2,081,747 discloses that certain amide derivatives of 4H-thieno[3,2-b]pyrroles and 4H-thieno[2,3-b]pyrroles are CCK antagonists and are useful in the treatment of gastric secretion disorders and in the regulation of appetite. The compounds disclosed in this document are disclaimed from the compound claims of the present invention.

U.S. Pat. No. 3,706,810 discloses that certain N-(aminoalkyl) derivatives of thieno[3,2-b]pyrrole-5-carboxamide are useful as analgesic and anti-depressant agents. The compounds disclosed in this document are disclaimed from the compound claims of the present invention.

U.S. Pat. No. 4,751,231 discloses that certain thieno[2,3-b]pyrrole-5-sulfonamides are useful in the treatment of elevated intraocular pressure and glaucoma. Certain amides are disclosed as intermediates. The compounds disclosed in this document are disclaimed from the compound claims of the present invention.

U.S. Pat. No. 4,794,120 discloses 4H-thieno[3,2-b]pyrrole-5-carboxylic acid hydrazide and 6H-thieno[2,3-b]pyrrole-5-carboxylic acid hydrazide as intermediates in the preparation of corresponding (5-nitro-2-furanyl)methylenehydrazides which are antibacterials, fungicides and protozoacides. The compounds disclosed in this document are disclaimed from the compound claims of the present invention.

Co-pending application EP 1088824 discloses that a compound of Formula A: a stereoisomer, pharmaceutically acceptable salt or prodrug thereof, or a pharmaceutically

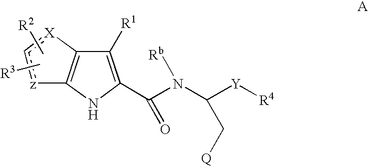

acceptable salt of the prodrug,

wherein

Q is aryl, substituted aryl, heteroaryl, or substituted heteroaryl;

each z and X are independently (C, CH or CH₂), N, O or S;

X¹ is NR^{a}, —CH₂—, O or S;

each - - - is independently a bond or is absent, provided that both - - - are not simultaneously bonds;

R¹ is hydrogen, halogen, —OC₁-C₈alkyl, —SC₁-C₈alkyl, —C₁-C₈alkyl, —CF₃, —NH₂—, —NHC₁-C₈alkyl, —N(C₁-C₈alkyl)₂, —NO₂, —CN, —CO₂H, —CO₂C₁-C₈alkyl, —C₂-C₈alkenyl, or —C₂-C₈alkynyl;

each R^{a} and R^{b} is independently hydrogen or —C₁-C₈alkyl;

Y is

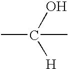

or absent;

R² and R³ are independently hydrogen, halogen, —C₁-C₈alkyl, —CN, —C≡C—Si(CH₃)₃, —OC₁-C₈alkyl, —SC₁-C₈alkyl, —CF₃, —NH₂, —NHC₁-C₈alkyl, —N(C₁-C₈alkyl)₂, —NO₂, —CO₂H, —CO₂C₁-C₈alkyl, —C₂-C₈alkenyl, or —C₂-C₈alkynyl, or R² and R³ together with the atoms on the ring to which they are attached form a five or six membered ring containing from 0 to 3 heteroatoms and from 0 to 2 double bonds;

R⁴ is —C(═O)-A;

A is —NR^{d}R^{d}, —NR^{a}CH₂CH₂OR^{a},

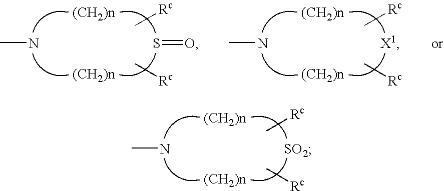

each R^{d} is independently hydrogen, C₁-C₈alkyl, C₁-C₈alkoxy, aryl, substituted aryl, heteroaryl, or substituted heteroaryl;

each R^{c} is independently hydrogen, —C(═O)OR, —OR^{a}, —SR^{a}, or NR^{a}R^{a}; and each n is independently 1-3,

are useful in treating diabetes, insulin resistance, diabetic neuropathy, diabetic nephropathy, diabetic retinopathy, cataracts, hyperglycemia, hypercholesterolemia, hypertension, hyperinsulinemia, hyperlipidemia, atherosclerosis, or tissue ischemia. These compounds are disclaimed from the present application.

The heterocyclic amides of the present invention possess glycogen phosphorylase inhibitory activity and accordingly are expected to be of use in the treatment of type 2 diabetes, insulin resistance, syndrome X, hyperinsulinaemia, hyperglucagonaemnia, cardiac ischaemia and obesity, particularly type 2 diabetes.

The present invention provides a compound of formula (I):

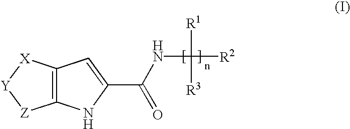

wherein:

—X-Y-Z- is selected from —S—CR⁴═CR⁵—, —CR⁴═CR⁵—S—, —O—CR⁴═CR⁵—, —CR⁴═CR⁵—O, —N═CR⁴S—, —S—CR⁴═N—, —NR⁶—CR⁴═CR⁵— and —CR═CR⁵—NR⁶—;

wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;

R⁶ is hydrogen or C₁₋₆alkyl;

R¹ is selected from hydrogen, halo, nitro, cyano, hydroxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₄alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, C₃₋₈cycloalkylC₁₋₆alkyl, aryl, arylC₁₋₆alkyl, heterocyclic group and (heterocyclic group)C₁₋₆alkyl; wherein R¹ may be optionally substituted on carbon by one or more groups selected from P and wherein if said heterocyclic group contains an —NH— moiety that nitrogen may be optionally substituted by a group selected from R;

R² is selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₆alkyl)₂amino, C₁₋₆alkanoylamino, N-(C₁₋₆alkyl)carbamoyl, N,N-(C₁₋₄alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, sulphamoylamino, N-(C₁₋₆alkyl)sulphamoylamino, N,N-(C₁₋₆alkyl)₂sulphamoylamino, C₁₋₆alkylsulphonylamino, C₁₋₆alkylsulphonylaminocarbonyl, C₁₋₆alkylsulphonyl-N-(C₁₋₆alkyl)amino and a group -E-F-G-H;

wherein E and G are independently selected from a direct bond, —O—, —S—, —SO—, —SO₂—, —OC(O)—, —C(O)O—, —C(O)—, —NR^{a}, —NR^{a}C(O)—, —C(O)NR^{a}—, —SO₂NR^{a}—, —NR^{a}SO₂—, —NR^{a}C(O)NR^{b}—, —OC(O)NR^{a}—, —NR^{a}C(O)O—, —NR^{a}SO₂NR^{b}—, —SO₂NR^{a}C(O)— and —C(O)NR^{a}SO₂—; wherein R^{a} and R^{b} are independently selected from hydrogen or C₁₋₆alkyl which is optionally substituted by a group V;

F is C₁₋₆alkylene optionally substituted by one or more Q or a direct bond;

H is selected from aryl, C₃₋₈cycloalkyl and heterocyclic group; wherein H may be optionally substituted on carbon by one or more groups selected from S and wherein if said heterocyclic group contains an —NH— moiety that nitrogen may be optionally substituted by a group selected from T;

R³ is hydrogen or C₁₋₆alkyl;

n is selected from 0-4; wherein the values of R¹ may be the same or different; and wherein the values of R³ may be the same or different;

P, S and Q are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein P, S and Q may be optionally and independently substituted on carbon by one or more groups selected from V and wherein if said heterocyclic group contains an —NH— moiety that nitrogen may be optionally substituted by a group selected from U;

V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl, and 4-hydroxypiperidinocarbonyl;

R, T and U are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)carbamoyl, phenyl, benzyl, benzyloxycarbonyl, benzoyl and phenylsulphonyl wherein R, T and U may be optionally and independently substituted on carbon by one or more groups selected from V;

or a pharmaceutically acceptable salt or an in vivo hydrolysable ester thereof;

with the provisos:

i) when —X-Y-Z- is —S—CH═CH—, R²—(CR¹R³)ₙ— cannot be amino, 1-phenyl-5-methyl-1H-1,5-benzodiazepine-2,4 (3H,5H)dion-3-yl, 1-methyl-5-phenyl-2-oxo-2,3-dihydro-1H-benzo(E)(1,4)diazepin-3-yl, 2-(4-phenyl-1,2,5,6-tetrahydropyrid-1-yl)ethyl, 3-(4-phenyl-1,2,5,6-tetrahydropyrid-1-yl)propyl, 2-(4-phenylpiperazin-1-yl)ethyl, 2-(N-methylamino)ethyl, 2-morpholinoethyl or 2-(N-methyl-N-benzylamino)ethyl;

ii) when —X-Y-Z- is —CH═CH—S—, R²—(CR¹R³)ₙ— cannot be amino or 1-methyl-5-phenyl-2-oxo-2,3-dihydro-1H-benzo(E)(1,4)diazepin-3-yl;

iii) when —X-Y-Z- is —CH═C(SO₂NH₂)—S—, R²—(CR¹R³)ₙ— cannot be methyl or isobutyl; and

iv) when —X-Y-Z- is as initially defined, n is 1, R¹ is arylmethyl, substituted arylmethyl, (heterocyclic group)methyl and substituted (heterocyclic group)methyl and R³ is hydrogen then R² is not a group —C(═O)-A or a group —CH(OH)—C(═O)-A in which A is NR^{d}R^{d}, —NR^{a}CH₂CH₂OR^{a}, or

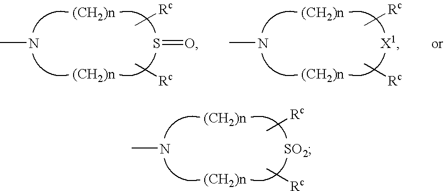

each R^{a} and R^{b} is independently hydrogen or —C₁-C₈alkyl;

each R^{d} is independently hydrogen, C₁-C₈alkyl, C₁-C₈alkoxy, aryl, substituted aryl, heteroaryl, or substituted heteroaryl;

each R^{c} is independently hydrogen, —C(═O)OR^{a}, —OR^{a}, SR^{a}, or NR^{a}R^{a}; and each n is independently 1-3, and

X¹ is NR^{a}, —CH₂,O or S.

In another aspect the present invention provides a compound of formula (I):

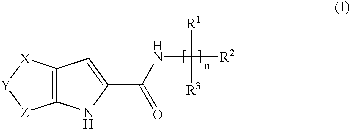

wherein:

—X-Y-Z- is selected from —S—CR⁴═CR⁵—, —CR⁴═CR⁵—S—, —O—CR⁴═CR⁵—, —CR⁴═CR⁵—O—, —N═CR⁴—S—, —S—CR⁴═N—, —NR⁶—CR⁴═CR⁵— and —CR⁴═CR⁵—NR⁶—;

wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy; C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;

R⁶ is hydrogen or C₁₋₆alkyl;

R¹ is selected from hydrogen, halo, nitro, cyano, hydroxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₄alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, C₃₋₈cycloalkylC₁₋₆alkyl, aryl, arylC₁₋₆alkyl, heterocyclic group and (heterocyclic group)C₁₋₆alkyl; wherein R¹ may be optionally substituted on carbon by one or more groups selected from P and wherein if said heterocyclic group contains an —NH— moiety that nitrogen may be optionally substituted by a group selected from R;

R² is selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₄alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, sulphamoylamino, N-(C₁₋₆alkyl)sulphamoylamino, N,N-(C₁₋₆alkyl)₂sulphamoylamino, C₁₋₆alkylsulphonylamino, C₁₋₆alkylsulphonylaminocarbonyl, C₁₋₆alkylsulphonyl-N-(C₁₋₆alkyl)amino and a group -E-F-G-H;

wherein E and G are independently selected from a direct bond, —O—, —S—, —SO—, —SO₂—, —OC(O)—, —C(O)O—, —C(O)—, —NR^{a}—, —NR^{a}C(O)—, —C(O)NR^{a}—, —SO₂NR^{a}—, —NR^{a}SO₂—, —NR^{a}C(O)NR^{b}—, —OC(O)NR^{a}—, —NR^{a}C(O)O—, —NR^{a}SO₂NR^{b}—, —SO₂NR^{a}C(O)— and —C(O)NR^{a}SO₂—; wherein R^{a} and R^{b} are independently selected from hydrogen or C₁₋₆alkyl;

F is C₁₋₆alkylene optionally substituted by one or more Q or a direct bond;

H is selected from aryl, C₃₋₈cycloalkyl and heterocyclic group; wherein H may be optionally substituted on carbon by one or more groups selected from S and wherein if said heterocyclic group contains an —NH— moiety that nitrogen may be optionally substituted by a group selected from T;

R³ is hydrogen or C₁₋₆alkyl;

n is selected from 0-4; wherein the values of R¹ may be the same or different; and wherein the values of R³ may be the same or different;

P, S and Q are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein P, S and Q may be optionally and independently substituted on carbon by one or more groups selected from V and wherein if said heterocyclic group contains an —NH— moiety that nitrogen may be optionally substituted by a group selected from U;

V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

R, T and U are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)carbamoyl, phenyl, benzyl, benzyloxycarbonyl, benzoyl and phenylsulphonyl; or a pharmaceutically acceptable salt or an in vivo hydrolysable ester thereof; with the provisos: i) when —X-Y-Z- is —S—CH═CH—, R²—(CR¹R³)ₙ— cannot be amino, 1-phenyl-5-methyl-1H-1,5-benzodiazepine-2,4 (3H,5H)dion-3-yl, 1-methyl-5-phenyl-2-oxo-2,3-dihydro-1H-benzo(E)(1,4)diazepin-3-yl, 2-(4-phenyl-1,2,5,6-tetrahydropyrid-1-yl)ethyl, 3-(4-phenyl-1,2,5,6-tetrahydropyrid-1-yl)propyl, 2-(4-phenylpiperazin-1-yl)ethyl, 2-(N-methylamino)ethyl, 2-morpholinoethyl or 2-(N-methyl-N-benzylamino)ethyl; ii) when —X-Y-Z- is —CH═CH—S—, R²—(CR¹R³)ₙ— cannot be amino or 1-methyl-5-phenyl-2-oxo-2,3-dihydro-1H-benzo(E)(1,4)diazepin-3-yl; iii)when —X-Y-Z- is —CH═C(SO₂NH₂)—S—, R²—(CR¹R³)ₙ— cannot be methyl or isobutyl; and iv) when —X-Y-Z- is as initially defined, n is 1, R¹ is arylmethyl, substituted arylmethyl, (heterocyclic group)methyl and substituted (heterocyclic group)methyl and R³ is hydrogen then R² is not a group —C(═O)-A or a group —CH(OH)—C(═O)-A in which A is NR^{d}R^{d}, NR^{a}CH₂CH₂OR^{a}, or

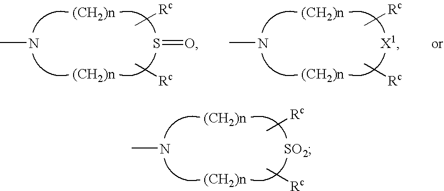

each R^{a} and R^{b} is independently hydrogen or —C₁-C₈alkyl;

each R^{d} is independently hydrogen, C₁-C₈alkyl, C₁-C₈alkoxy, aryl, substituted aryl, heteroaryl, or substituted heteroaryl;

each R^{c} is independently hydrogen, —C(═O)OR^{a}, —OR^{a}, —SR^{a}, or NR^{a}R^{a}; and each n is independently 1-3, and

X¹ is NR^{a}, —CH₁₂—, O or S.

In this specification the term “alkyl” includes both straight and branched chain alkyl groups but references to individual alkyl groups such as “propyl” are specific for the straight chain version only. For example, “C₁₋₆alkyl” includes C₁₋₄alkyl, propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals, for example “arylC₁₋₆alkyl” includes arylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclic group” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)-and a ring sulphur atom may be optionally oxidised to form the S-oxide(s). Examples and suitable values of the term “heterocyclic group” are morpholino, piperidyl, pyridyl, pyranyl, pyrrolyl, imidazolyl, thiazolyl, indolyl, quinolyl, thienyl, 1,3-benzodioxolyl, 1,3-dioxolanyl, thiadiazolyl, piperazinyl, isothiazolidinyl, 1,3,4-triazolyl, tetrazolyl, pyrrolidinyl, 2-oxazolidinonyl, 5-isoxazolonyl, benz-3-azepinyl, 1,4-benzodioxanyl, thiomorpholino, pyrrolinyl, homopiperazinyl, 3,5-dioxapiperidinyl, 3-pyrazolin-5-onyl, tetrahydropyranyl, benzimidazolyl, benzthiazolyl, imidazo[1,2-a]pyridyl, pyrimidyl, pyrazinyl, pyridazinyl, isoxazolyl, 4-pyridone, 1-isoquinolone, 2-pyrrolidone, 4-thiazolidone 2,3-dihydro-1,5-benzothiazepin-4 (5H)-one. Preferably a “heterocyclic group” is pyridyl, imidazolyl, thiazolyl, quinolyl, thienyl, 1,3-benzodioxolyl, 1,3-dioxolanyl, isothiazolidinyl, 1,3,4-triazolyl, tetrazolyl, 2-oxazolidinonyl, 5-isoxazolonyl, benz-3-azepinyl, hydantoinyl, 1,4-benzodioxanyl, thiomorpholino, 3-pyrazolin-5-onyl, benzimidazolyl, benzthiazolyl, imidazo[1,2-a]pyridyl, pyrimidyl, pyrazinyl, and 2,3-dihydro-1,5-benzothiazepin-4 (5H)-one

“Aryl” is a partially saturated or unsaturated, mono or bicyclic ring containing 4-12 carbon atoms, wherein a —CH₂— group can optionally be replaced by a —C(O)—. Preferably aryl is phenyl, naphthyl, 1,2,3,4-tetrahydronaphthyl (tetralinyl) or indanyl. More preferably aryl is phenyl, naphthyl or 1,2,3,4-tetrahydronaphthyl. Most preferably aryl is phenyl, or naphthyl.

An example of “C₁₋₆alkanoyloxy” is acetoxy. Examples of “C₁₋₆alkoxycarbonyl” include C₁₋₄alkoxycarbonyl, methoxycarbonyl, ethoxycarbonyl, n- and t-butoxycarbonyl. Examples of “C₁₋₆alkoxycarbonylamino” include methoxycarbonylamino, ethoxycarbonylamino, n- and t-butoxycarbonylamino. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include C₁₋₄alkylsulphonyl, methylthio, ethylthio, methylsulphinyl, ethylsulphinyl, mesyl and ethylsulphonyl. Examples of “C₁₋₆alkylsulphonylamino” include methylsulphonylamino, ethylsulphonylamino and propylsulphonylamino. Examples of “C₁₋₆alkylsulphonyl-N-(C₁₋₆alkyl)amino” include methylsulphonyl-N-methylamino, ethylsulphonyl-N-methylamino and propylsulphonyl-N-ethylamino. Examples of “C₁₋₆alkanoyl” include C₁₋₄alkanoyl, propionyl and acetyl. Examples of “N-(C₁₋₆alkyl)amino” include methylamino and ethylamino. Examples of “N,N-(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N-(C₁₋₆alkyl)sulphamoyl” are N-(methyl)sulphamoyl and N-(ethyl)sulphamoyl. Examples of “N-(C₁₋₆alkyl)₂sulphamoyl” are N,N-(dimethyl)sulphamoyl and N-(methyl)-N-(ethyl)sulphamoyl. Examples of “N-(C₁₋₆alkyl)carbamoyl” are N-(C₁₋₄alkyl)carbamoyl, methylaminocarbonyl and ethylaminocarbonyl. Examples of “N,N-(C₁₋₆alkyl)₂carbamoyl” are N,N-(C₁₋₄alkyl)carbamoyl, dimethylaminocarbonyl and methylethylaminocarbonyl. Examples of “C₃₋₈cycloalkyl ring” are cyclopropyl and cyclohexyl. Examples of “(heterocyclic group)C₁₋₆alkyl” include pyridylmethyl, 3-morpholinopropyl and 2-pyrimid-2-ylethyl. Examples of “C₃₋₈cycloalkylC₁₋₆cycloalkyl” include cyclopropylmethyl and 2-cyclohexylpropyl. “N-(C₁₋₆alkyl)sulphamoylamino” are N-(methyl)sulphamoylamino and N-(ethyl)sulphamoylamino. Examples of “N-(C₁₋₆alkyl)₂sulphamoylamino” are N,N-(dimethyl)sulphamoylamino and N-(methyl)-N-(ethyl)sulphamoylamino. Examples of “C₁₋₆alkylsulphonylanuinocarbonyl” include methylsulphonylaminocarbonyl, ethylsulphonylaminocarbonyl and propylsulphonylaminocarbonyl.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

An in vivo hydrolysable ester of a compound of the formula (I) containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

Some compounds of the formula (I) may have chiral centres and/or geometric isomeric centres (E- and Z-isomers), and it is to be understood that the invention encompasses all such optical, diastereoisomers and geometric isomers that possess glycogen phosphorylase inhibitory activity.

The invention relates to any and all tautomeric forms of the compounds of the formula (I) that possess glycogen phosphorylase inhibitory activity.

It is also to be understood that certain compounds of the formula (I) can exist in solvated as well as unsolvated forms such as, for example, hydrated forms. It is to be understood that the invention encompasses all such solvated forms which possess glycogen phosphorylase inhibitory activity.

Preferred values of R¹, R², R³, —X-Y-Z-and n are as follows. Such values may be used where appropriate with any of the definitions, claims or embodiments defined hereinbefore or hereinafter.

Preferably —X-Y-Z- is selected from —S—CR⁴═CR⁵—, —CR⁴CR⁵—S—, —O—CR⁴═CR⁵— and —N═CR⁴—S—.

More preferably —X-Y-Z- is selected from —S—CR⁴═CR⁵— and —CR⁴ CR⁵—S—.

In one aspect of the invention preferably —X-Y-Z- is selected from —S—CR⁴═CR⁵—.

In another aspect of the invention preferably —X-Y-Z- is selected from —CR⁴═CR⁵—S—.

Preferably R⁴ and R⁵ are independently selected from hydrogen, halo or C₁₋₆alkyl.

More preferably R⁴ and R⁵ are independently selected from hydrogen, chloro, bromo or methyl.

Particularly R⁴ and R⁵ are independently selected from hydrogen or chloro.

More particularly R⁴ and R⁵ are both chloro.

Preferably —X-Y-Z- is selected from —S—C(Cl)═C(Cl)—, —S—C(Cl)═CH—, —S—CH═C(Cl)—, —S—C(Br)═CH—, —S—CH═CH—, —CH═CH—S—, —O—CH═CH—, —N═C(Me)-S— and —S—CH═CCl—. More preferably —X-Y-Z- is selected from —S—C(Cl)═C(Cl)—, —S—C(Cl)═CH—, —S—CH═C(Cl)—, —S—C(Br)═CH—, —S—CH═CH—, —CH═CH—S—, —O—CH═CH— and —N—C(Me)-S—.

Most preferably —X-Y-Z- is selected from —S—C(Cl)═C(Cl)—, —S—C(Cl)═CH— and —S—CH═C(Cl)—.

Particularly —X-Y-Z- is selected from —S—C(Cl)═C(Cl)—.

In one aspect of the invention, preferably R⁶ is hydrogen.

In another aspect of the invention, preferably R⁶ is C₁₋₆alkyl.

Preferably R¹ is selected from hydrogen, hydroxy, C₁₋₆alkyl, C₁₋₆alkoxycarbonyl, arylC₁₋₆alkyl and (heterocyclic group)C₁₋₆alkyl; wherein R¹ may be optionally substituted on carbon by one or more groups selected from P; and

P is selected from hydroxy and C₁₋₆alkylsulphonyl-N-(C₁₋₆alkyl)amino.

More preferably R¹ is selected from hydrogen, hydroxy, methyl, methoxycarbonyl, benzyl and imidazol-4-ylmethyl; wherein R¹ may be optionally substituted on carbon by one or more groups selected from P; and

P is selected from hydroxy and mesyl-N-(methyl)amino.

Particularly R¹ is selected from hydrogen, hydroxy, methyl, methoxycarbonyl, mesyl-N-(methyl)aminomethyl, benzyl, hydroxymethyl and imidazol-4-ylmethyl.

More particularly R¹ is selected from hydrogen, mesyl-N-(methyl)aminomethyl or benzyl.

Preferably R² is selected from N,N-(C₁₋₄alkyl)₂carbamoyl, N,N-(C₁₋₆alkyl)₂sulphamoylamino, C₁₋₆alkylsulphonylaminocarbonyl and a group -E-F-G-H;

wherein E and G are independently selected from a direct bond, —O—, —S—, —C(O)—, —NR^{a}—, —C(O)NR^{a}—, NR^{a}SO₂— and —NR^{a}C(O)O—; wherein R^{a} is hydrogen;

F is C₁₋₆alkylene optionally substituted by one or more Q or a direct bond;

H is selected from aryl, C₃₋₈cycloalkyl and heterocyclic group; wherein H may be optionally substituted on carbon by one or more groups selected from S and wherein if said heterocyclic group contains an —NH— moiety that nitrogen may be optionally substituted by a group selected from T;

S is selected from halo, hydroxy, trifluoromethyl, sulphamoyl, ureido, C₁₋₆alkyl, C₁₋₆alkoxy, N,N-(C₁₋₆alkyl)₂amino, C₁₋₆alkanoylamino and aryl; wherein S may be optionally substituted on carbon by one or more groups selected from V;

Q is hydroxy;

V is carbamoyl; and

T is independently selected from C₁₋₄alkyl or phenyl.

More preferably R² is selected from N,N-dimethylcarbamoyl, N,N-dimethylsulphamoylamino, mesylaminocarbonyl and a group -E-F-G-H;

wherein E and G are independently selected from a direct bond, —O—, —S—, —C(O)—, —NR^{a}—, —C(O)NR^{a}—, —NR^{a}SO₂— and —NR^{a}C(O)O—; wherein R^{a} is hydrogen;

F is methylene optionally substituted by one or more Q or a direct bond;

H is selected from phenyl, naphthyl, cyclopropyl, thiomorpholino, pyridyl, thiazolyl, isothiazolyl, morpholinyl, 2,3-dihydro-1,5-benzothiazepin-4 (5H)-onyl, 5-oxo-3-pyrazolinyl, 2-oxazolidinonyl, 5-hydroxy-1,3,4,5-tetrahydro-benzo[b]azepin-2-onyl, 5-oxo-2-isoxazolinyl, imidazo[1,2-a]pyridinyl, benzothiazolyl, 2,5-dioxoimidazolidinyl, pyrazinyl, pyridazinyl, imidazolyl, benzimidazolyl, tetrazolyl, quinolyl, 1,3-dioxolanyl and thienyl; wherein H may be optionally substituted on carbon by one or more groups selected from S and wherein if a heterocyclic group contains an —NH— moiety that nitrogen may be optionally substituted by a group selected from T;

S is selected from fluoro, chloro, hydroxy, trifluoromethyl, sulphamoyl, ureido, methyl, ethyl, methoxy, N,N-dimethylamino, acetamido and phenyl; wherein S may be optionally substituted on carbon by one or more groups selected from V;

Q is hydroxy;

V is carbamoyl; and

T is independently selected from methyl or phenyl.

Particularly R² is selected from N,N-dimethylcarbamoyl, N,N-dimethylsulphamoylamino, mesylaminocarbonyl, 2-methoxyphenyl, phenoxy, 2-phenylcyclopropyl, thien-2-yl, 4-fluorophenyl, benzoyl, thiomorpholino, anilinocarbonyl, pyrid-2-ylamino, thiazol-2-yl, benzylsulphonylamino, 2,3-dihydro-1,5-benzothiazepin-4 (5H)-one-3-yl, 1-phenyl-2,3-dimethyl-5-oxo-3-pyrazolin-4-yl, 3-phenyl-2-oxazolidinon-5-yl, 5-hydroxy-1,3,4,5-tetrahydrobenzo[b]azepin-2-onyl, 3-phenyl-5-oxo-2-isoxazolin-4-yl, imidazo[1,2-a]pyridin-2-yl, benzothiazol-2-yl, 2,5-dioxoimidazolidin-3-yl, naphth-2-ylaminocarbonyl, phenyl, 4-sulphamoylphenethyl, 4-(N,N-dimethylamino)phenyl, 4-sulphamoylphenyl, anilino, 4-hydroxyphenyl, quinolin-3-yl, 4-chlorophenyl, 2-methoxypyrid-5-yl, 3-methylisothiazol-5-yl, 3-trifluoromethylpyrid-2-yl, tetrazol-5-yl, benzyloxycarbonylamino, benzimidazol-2-yl, 2-trifluoromethylpyrid-5-yl, pyridazin-2-yl, pyridazin-3-yloxy, pyrid-2-yl, imidazol-5-yl, 4-acetamidophenoxy, 2-ureidothiazol-4-yl, benzylthio, 2-phenyl-1,3-dioxolan-2-yl, 4-carbamoylmethylphenoxy, (N-benzylcarbamoylmethyl), phenethyl, 3-phenylpropyl, [2-(2-hydroxyphenyl)ethyl], -(α,α-dimethylphenethyl), (1-phenylcyclobutyl)methyl), (β-methylphenethyl), (1,2,3,4-tetrahydronaphth-2-yl), benzyl, (N-benzyl-N-methylcarbamoylmethyl), (N-methyl-N-phenylcarbamoylmethyl), [N-(2-cyanoethyl)-N-phenylcarbamoylmethyl], [N-(4-methoxyphenyl)carbamoylmethyl], [N-(4-fluorophenyl)carbamoylmethyl], [N-(4-nitrophenyl)carbamoylmethyl], [N-(2,6-dimethylphenyl)carbamoylmethyl], [N-methyl-N-(4-methylphenyl)carbamoylmethyl], [N-methyl-N-(3-methylphenyl)carbamoylmethyl], [N-(3-chlorophenyl) N-methylcarbamoylmethyl], [N-(2-hydroxyethyl)-N-phenylcarbamoylmethyl], [N-(1,1-dimethyl-2-hydroxyethyl)carbamoylmethyl], [N-(2-hydroxyethyl)-N-methylcarbamoyl-methyl], [N-(2-hydroxyethyl)carbamoylmethyl], [N-(3-hydroxypropyl)carbamoylmethyl], [N-(4-hydroxybutyl)carbamoylmethyl], {N-[bis(hydroxymethyl)methyl]carbamoylmethyl}, [N-(2,3-dihydroxypropyl)carbamoylmethyl], [N-(4-hydroxymethylphenyl)-carbamoylmethyl], [N-(5-isoquinolyl)carbamoylmethyl], [N-(3-hydroxymethy)lphenyl]-carbamoylmethyl], {N-[4-(2-hydroxyethyl)phenyl]carbamoyl-methyl}, [N-(2,4-difluorophenyl)-N-methylcarbamoylmethyl], [(1,2,3,4-tetrahydro-1-quinolyl)carbonyl-methyl], [N-(2-cyanoethyl)-N-methylcarbamoylmethyl], [N-(4-hydroxypiperidino)-carbamoylmethyl], (N-cyclopentylcarbamoylmethyl), (N-isopropyl-carbamoylmethyl), (N-isopropyl-N-methylcarbamoylmethyl), (thiomorpholin-carbonylmethyl), (morpholino-carbonylmethyl), [(1,1-dioxothiomorpholino)carbonyl-methyl], [(1-oxothiomorpholino)-carbonylmethyl], (2-indanyl), (benz[1,2]oxazol-3-ylmethyl), {2-[2-(hydroxymethyl)phenyl]-ethyl}, (4-phenylisoxazol-3-ylmethyl), {2-[2-(2-morpholino-ethoxy)phenyl]ethyl}, {2-[2-(methoxycarbonyl-methoxy)phenyl]ethyl}, {2-[2-(carboxy-methoxy)phenyl]ethyl}, [2-(3-methoxyphenyl)ethyl], (2-oxo-1,2,3,4-tetrahydroquinol-3-yl), {2-[2-(2-methoxyethoxy)phenyl]ethyl}, {2-[2-(carbamoylmethoxy)phenyl]ethyl}, {2-[2-(N-methylcarbamoylmethoxy)phenyl]ethyl}, {2-[2-(N,N-dimethylcarbamoylmethoxy)phenyl]ethyl}, 2-[2-(morpholinocarbonylmethoxy)-phenyl]ethyl, 2-[2-(N-benzylcarbamoylmethoxy)phenyl]ethyl, 2-[2-(4-hydroxypiperidinocarbonylmethoxy)-phenyl]ethyl, [1-(5-ethoxycarbonyl-1,3,4-oxadiazo-2-yl)-2-phenylethyl], [1-(4-methoxycarbonyl-oxazo-5-yl)-2-phenylethyl], [2-phenylethyl-1-(pyrid-3-yl)], [2-phenylethyl-1-(3-phenyl-1,2,4-oxadiazo-5-yl)], (1-hydroxyindan-2-yl), [(1S,2S)-2-indan-1-ol], [(1R,2R)-2-indan-1-ol], (3-indanyl), (1-hydroxy-1,2,3,4-tetrahydronaphth-2-yl), (6-fluoro-1-hydroxyindan-2-yl), (7-methoxy-1-oxo-1,2,3,4-tetrahydronaphth-2-yl), -(3-methylisoxazol-5-yl)methyl], (4-hydroxy-1,1-dioxotetrahydrothiophen-3-yl), -{N-methyl-N-[(1-oxo-1,2,3,4-tetrahydronaphth-2-yl)methyl]carbamoylmethyl}, (3-methylisoxazol-5-yl)methyl], (4-hydroxy-1,1-dioxotetrahydrothiophen-3-yl), {N-methyl-N-[(1-oxo-1,2,3,4-tetrahydronaphth-2-yl)methyl]carbamoylmethyl}, (2-oxo-1,2,3,4-tetrahydroquinol-3-yl), (1,2,3,4-tetrahydroquinol-3-yl), (1-methyl-2-oxo-1,2,3,4-tetrahydroquinol-3-yl), (3-oxo-2,3,4,5-tetrahydro-1H-benz[2]azepin-4-yl), (1-methoxyindan-2-yl), {1-[N-(1,1-dimethylethoxy)carbonylamino]indan-2-yl}, (1-aminoindan-2-yl)], (1-acetamidoindan-2-yl), [1-(methanesulphonamido)indan-2-yl], [1-(methylamino)indan-2-yl], or [1-(N-methylacetamido)indan-2-yl].

More particularly R² is selected from N,N-dimethylcarbamoyl, N,N-dimethylsulphamoylamino, mesylaminocarbonyl, 2-methoxyphenyl, phenoxy, 2-phenylcyclopropyl, thien-2-yl, 4-fluorophenyl, benzoyl, thiomorpholino, anilinocarbonyl, pyrid-2-ylamino, thiazol-2-yl, benzylsulphonylamino, 2,3-dihydro-1,5-benzothiazepin-4 (5H)-one-3-yl, 1-phenyl-2,3-dimethyl-5-oxo-3-pyrazolin-4-yl, 3-phenyl-2-oxazolidinon-5-yl, 5-hydroxy-1,3,4,5-tetrahydro-benzo[b]azepin-2-onyl, 3-phenyl-5-oxo-2-isoxazolin-4-yl, imidazo[1,2-a]pyridin-2-yl, benzothiazol-2-yl, 2,5-dioxoimidazolidin-3-yl, naphth-2-ylaminocarbonyl, phenyl, 4-sulphamoylphenethyl, 4-(N; N-dimethylamino)phenyl, 4-sulphamoylphenyl, anilino, 4-hydroxyphenyl, quinolin-3-yl, 4-chlorophenyl, 2-methoxypyrid-5-yl, 3-methylisothiazol-5-yl, 3-trifluoromethylpyrid-2-yl, tetrazol-5-yl, benzyloxycarbonylamino, benzimidazol-2-yl, 2-trifluoromethylpyrid-5-yl, pyridazin-2-yl, pyridazin-3-yloxy, pyrid-2-yl, imidazol-5-yl, 4-acetamidophenoxy, 2-ureidothiazol-4-yl, benzylthio, 2-phenyl-1,3-dioxolan-2-yl and 4-carbamoylmethylphenoxy.

Most particularly R² is selected from N,N-dimethylcarbamoyl, phenoxy, 2-phenylcyclopropyl, thien-2-yl, 4-fluorophenyl, benzoyl, thiomorpholino, anilinocarbonyl, pyrid-2-ylamino or thiazol-2-yl.

Preferably R³ is hydrogen.

Preferably n is selected from 0-3; wherein the values of R¹ may be the same or different; and wherein the values of R³ may be the same or different.

More preferably n is selected from 0-2; wherein the values of R¹ may be the same or different; and wherein the values of R³ may be the same or different.

In one aspect of the invention preferably n is 2; wherein the values of R¹ may be the same or different; and wherein the values of R³ may be the same or different.

In one aspect of the invention preferably n is 1.

In one aspect of the invention preferably n is 0.

In another aspect of the invention, preferred compounds of the invention are any one of the Examples or a pharmaceutically acceptable salt or an in vivo hydrolysable ester thereof.

In another aspect the present invention provides a compound of formula (I) (as depicted above) wherein:

—X-Y-Z- is selected from —S—CR⁴═CR⁵—, —CR⁴═CR⁵—S—, —O—CR⁴═CR⁵— and —N═CR⁴—S—;

R⁴ and R⁵ are independently selected from hydrogen, halo or C₁₋₆alkyl;

R¹ is selected from hydrogen, hydroxy, C₁₋₆alkyl, C₁₋₆alkoxycarbonyl, arylC₁₋₆alkyl and (heterocyclic group)C₁₋₆alkyl; wherein R¹ may be optionally substituted on carbon by one or more groups selected from P; and

P is selected from hydroxy and C₁₋₆alkylsulphonyl-N-(C₁₋₆alkyl)amino;

R² is selected from N,N-(C₁₋₄alkyl)₂carbamoyl, N,N-(C₁₋₆alkyl)₂sulphamoylamino, C₁₋₆alkylsulphonylaminocarbonyl and a group -E-F-G-H;

wherein E and G are independently selected from a direct bond, —O—, —S—, —C(O)—, —NR^{a}—, —C(O)NR^{a}—, —NR^{a}SO₂— and —NR^{a}C(O)O—; wherein R^{a} is hydrogen;

F is C₁₋₆alkylene optionally substituted by one or more Q or a direct bond;

H is selected from aryl, C₃₋₈cycloalkyl and heterocyclic group; wherein H may be optionally substituted on carbon by one or more groups selected from S and wherein if said heterocyclic group contains an —NH— moiety that nitrogen may be optionally substituted by a group selected from T;

S is selected from halo, hydroxy, trifluoromethyl, sulphamoyl, ureido, C₁₋₆alkyl, C₁₋₆alkoxy, N,N-(C₁₋₆alkyl)₂amino, C₁₋₆alkanoylamino and aryl; wherein S may be optionally substituted on carbon by one or more groups selected from V;

Q is hydroxy;

V is carbamoyl; and

T is independently selected from C₁₋₄alkyl or phenyl;

R³ is hydrogen;

n is selected from 0-3; wherein the values of R¹ may be the same or different;

or a pharmaceutically acceptable salt or an ii? vivo hydrolysable ester thereof;

with the provisos:

i) when —X-Y-Z- is —S—CH═CH—, R²—(CR¹R³)ₙ— cannot be 1-phenyl-5-methyl-1H-1,5-benzodiazepine-2,4 (3H,5H)dion-3-yl, 1-methyl-5-phenyl-2-oxo-2,3-dihydro-1H-benzo(E)(1,4)diazepin-3-yl, 2-(4-phenyl-1,2,5,6-tetrahydropyrid-1-yl)ethyl, 3-(4-phenyl-1,2,5,6-tetrahydropyrid-1-yl)propyl, 2-(4-phenylpiperazin-1-yl)ethyl or 2-morpholinoethyl;

ii) when —X-Y-Z- is —CH═CH—S—, R²—(CR¹R³)ₙ— cannot be 1-methyl-5-phenyl-2-oxo-2,3-dihydro-1H-benzo(E)(1,4)diazepin-3-yl; and

iii) when —X-Y-Z- is as initially defined, n is 1, R¹ is arylmethyl, substituted arylmethyl, (heterocyclic group)methyl and substituted (heterocyclic group)methyl and R³ is hydrogen then R² is not a group —C(═O)-A or a group —CH(OH)—C(═O)-A in which A is NR^{d}R^{d}, —NR^{a}CH₂CH₂OR^{a}, or

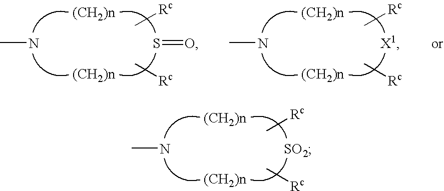

each R^{a} and R^{b} is independently hydrogen or —C₁-C₈alkyl;

each R^{d} is independently hydrogen, C₁-C₈alkyl, C₁-C₈alkoxy, aryl, substituted aryl, heteroaryl, or substituted heteroaryl;

each R^{c} is independently hydrogen, —C(═O)OR^{a}, —OR^{a}, —SR^{a}, or —NR^{a}R^{a}; and each n is independently 1-3, and

X¹ is NR^{a}, CH₂, or S.

In yet another aspect the present invention provides a compound of formula (I) (as depicted above) wherein:

—X-Y-Z- is selected from —S—C(Cl)═C(Cl)—, —S—C(Cl)═CH—, —S—CH═C(Cl)—, —S—C(Br)═CH—, —S—CH═CH—, —CH═CH—S—, —O—CH═CH— and —N═C(Me)—S—;

R¹ is selected from hydrogen, hydroxy, methyl, methoxycarbonyl, mesyl-N-(methyl)aminomethyl and hydroxymethyl;

R² is selected from N,N-dimethylcarbamoyl, N,N-dimethylsulphamoylamino, mesylaminocarbonyl, 2-methoxyphenyl, phenoxy, 2-phenylcyclopropyl, thien-2-yl, 4-fluorophenyl, benzoyl, thiomorpholino, anilinocarbonyl, pyrid-2-ylamino, thiazol-2-yl, benzylsulphonylamino, 2,3-dihydro-1,5-benzothiazepin-4 (5H)-one-3-yl, 1-phenyl-2,3-dimethyl-5-oxo-3-pyrazolin-4-yl, 3-phenyl-2-oxazolidinon-5-yl, 5-hydroxy-1,3,4,5-tetrahydrobenzo[B]azepin-2-onyl, 3-phenyl-5-oxo-2-isoxazolin-4-yl, imidazo[1,2-a]pyridin-2-yl, benzothiazol-2-yl, 2,5-dioxoimidazolidin-3-yl, naphth-2-ylaminocarbonyl, phenyl, 4-sulphamoylphenethyl, 4-(N,N-dimethylamino)phenyl, 4-sulphamoylphenyl, anilino, 4-hydroxyphenyl, quinolin-3-yl, 4-chlorophenyl, 2-methoxypyrid-5-yl, 3-methylisothiazol-5-yl, 3-trifluoromethylpyrid-2-yl, tetrazol-5-yl, benzyloxycarbonylamino, benzimidazol-2-yl, 2-trifluoromethylpyrid-5-yl, pyridazin-2-yl, pyridazin-3-yloxy, pyrid-2-yl, imidazol-5-yl, 4-acetamidophenoxy, 2-ureidothiazol-4-yl, benzylthio, 2-phenyl-1,3-dioxolan-2-yl and 4-carbamoylmethylphenoxy;

R³ is hydrogen; and

n is selected from 0-3; wherein the values of R¹ may be the same or different; or a pharmaceutically acceptable salt or an in vivo hydrolysable ester thereof.

In a first preferred aspect the present invention provides a compound of formula (I) (as depicted above) wherein:

—X-Y-Z- is selected from —S—CR═CR⁵— or —CR⁴═CR⁵—S—;

wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;

n is 0;

R² is a group -E-F-G-H;

wherein E, F and G are each a direct bond;

H is a C₃₋₁₂cycloalkyl which is optionally fused to a benz ring wherein H may be optionally substituted on carbon by one or more groups S which are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic groups; wherein S may be optionally substituted on carbon by one or more groups selected from V;

V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl, and 4-hydroxypiperidinocarbonyl;

or a pharmaceutically acceptable salt thereof.

Preferred values of R², R⁴, and R⁵ are as follows. Such values may be used where appropriate with any of the definitions, claims or embodiments defined hereinbefore or hereinafter.

In this first preferred aspect preferably R⁴ and R⁵ are independently selected from hydrogen, halo or C₁₋₆alkyl.

In this first preferred aspect preferably H is indanyl, 1,2,3,4-tetrahydronaphthyl or cyclopropyl. More preferably H is indanyl or 1,2,3,4-tetrahydronaphthyl. Most preferably H is indanyl.

In this first preferred aspect preferably S is independently selected from from halo, nitro, cyano, hydroxy, amino, carboxy, carbamoyl, C₁₋₆alkyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoyl amino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, C₃₋₈cycloalkyl and aryl. More preferably S is selected from hydroxy, amino, C₁₋₆alkoxy and C₁₋₆alkoxycarbonylamino.

In a second preferred aspect the present invention provides a compound of formula (I) (as depicted above) wherein:

—X-Y-Z- is selected from —S—CR⁴═CR⁵— or —CR⁴═CR⁵—S—;

wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;

n is 0;

R² is a group -E-F-G-H;

wherein E, F and G are each a direct bond; and

H is a cyclic amide of formula

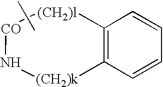

in which k is 0, 1, 2 or 3 and l is 0, 1, 2 or 3 such that the sum of k and l is 2 or 3 and wherein one of the carbon atoms governed by k or l may be replaced by sulphur and wherein H is optionally substituted on carbon by one or more groups selected from S and may be independently optionally substituted on nitrogen by a group selected from T;

S is selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein S may be optionally and independently substituted on carbon by one or more groups selected from V and wherein if said heterocyclic group contains an —NH— moiety that nitrogen may be optionally substituted by a group selected from U;

T and U are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)carbamoyl, phenyl, benzyl, benzyloxycarbonyl, benzoyl and phenylsulphonyl wherein T and U may be optionally and independently substituted on carbon by one or more groups selected from V;

V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl and 4-hydroxypiperidinocarbonyl;

or a pharmaceutically acceptable salt or an in vivo hydrolysable ester thereof.

Preferred values of R⁴, R⁵ and H are as follows. Such values may be used where appropriate with any of the definitions, claims or embodiments defined hereinbefore or hereinafter.

In this second preferred aspect preferably R⁴ and R⁵ are independently selected from hydrogen, halo or C₁₋₆alkyl.

In this second preferred aspect preferably H is 1,2,3,4-tetrahydroquinolyl, 2-oxo-1,2,3,4-tetrahydroquinolyl, 4-oxo-2,3,4,5-tetrahydrobenz[1,5]thiazepin-3-yl, 2-oxo-2,3,4,5-tetrahydro-1H-benz[b]azepinyl, 2,3,4,5-tetrahydro-1H-benz[b]azepinyl or 3-oxo-2,3,4,5-tetrahydro-1H-benz[c]azepinyl each optionally substituted on carbon by one or more groups selected from S wherein S is selected from hydroxy, C₁₋₆alkyl or C₁₋₆alkoxy, and each independently optionally substituted on nitrogen by a group selected from T wherein T is selected from C₁₋₄alkyl or C₁₋₄alkanoyl.

More preferably H is 2-oxo-1,2,3,4-tetrahydroquinol-3-yl, 1-methyl-2-oxo-1,2,3,4-tetrahydroquinol-3-yl, 4-oxo-2,3,4,5-tetrahydrobenz[1,5]thiazepin-3-yl, 5-hydroxy-2-oxo-2,3,4,5-tetrahydro-1H-benz[b]azepin-4-yl, 2-oxo-2,3,4,5-tetrahydro-1H-benz[b]azepin-3-yl or 3-oxo-2,3,4,5-tetrahydro-1H-benz[c]azepin-4-yl.

In a third preferred aspect the present invention provides a compound of formula (I) (as depicted above) wherein:

—X-Y-Z- is selected from —S—CR⁴═CR⁵— or —CR⁴═CR⁵—S—; wherein R⁴ and R⁵ are independently selected from hydrogen, halo or C₁₋₆alkyl.

n is 1;

R¹ is hydrogen or arylC₁₋₆alkyl;

R² is selected from a group -E-F-G-H;

wherein E, F and G are each a direct bond;

H is an unsaturated five membered heterocyclic group containing at least one nitrogen atom and one or two ring atoms selected from oxygen and sulphur and wherein H may be optionally substituted on carbon by one or more groups S which are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl and aryl groups;

R³ is hydrogen or C₁₋₆alkyl;

or a pharmaceutically acceptable salt thereof.

Preferred values of R¹, R³ and H are as follows. Such values may be used where appropriate with any of the definitions, claims or embodiments defined hereinbefore or hereinafter.

In this third preferred aspect preferably R¹ is selected from hydrogen or benzyl and more preferably benzyl.

In this third preferred aspect preferably R³ is hydrogen.

In this third preferred aspect preferably H is 1,3,4-oxadiazolyl, isoxazolyl, oxazolyl or 1,2,4-oxadiazolyl. More preferably H is 5-ethoxycarbonyl-1,3,4-oxadiazol-2-yl, 4-phenylisoxazol-3-yl, 3-phenyl-1,2,4-oxadiazol-5-yl, 4-methoxycarbonyloxazol-5-yl or 3-methylisoxazol-5-yl.

In this third preferred aspect preferably H may be optionally substituted on carbon by one or more groups S which are independently selected from halo, carboxy, C₁₋₆alkyl, C₁₋₆alkoxy, C₁₋₆alkanoyloxy, N-(C₁₋₆alkyl)amino, N,N-(C₁₋₆alkyl)₂amino, C₁₋₆alkanoylamino, C₁₋₆alkoxycarbonyl, C₃₋₈cycloalkyl and aryl groups. Preferably S is C₁₋₆alkoxy, C₁₋₆alkoxycarbonyl or phenyl.

In a fourth preferred aspect the present invention provides a compound of formula (I) (as depicted above) wherein:

—X-Y-Z- is selected from —S—CR⁴═CR⁵— or —CR⁴═CR⁵—S—;

wherein R⁴ and R⁵ are independently selected from hydrogen, halo or C₁₋₆alkyl.

n is 0;

R² is a group -E-F-G-H;

wherein F is a direct bond;

F is methylene;

wherein G is —C(O)NR^{a}—, wherein R^{a} is selected from hydrogen or C₁₋₆alkyl which is optionally substituted by a group V;

H is aryl which may be optionally substituted on carbon by one or more groups selected from S;

S is selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein S may be optionally and independently substituted on carbon by one or more groups selected from V;

V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N N-dimethylsulphamoyl, N N-diethylsulphamoyl, N-methyl-N-ethylsulphamoyl, morpholino, morpholinocarbonyl, N-benzylcarbamoyl, and 4-hydroxypiperidinocarbonyl;

or a pharmaceutically acceptable salt thereof.

Preferred values of R¹, R², R³, —X-Y-Z-and n are as follows. Such values may be used where appropriate with any of the definitions, claims or embodiments defined hereinbefore or hereinafter.

In this fourth preferred aspect preferably H is aryl.

In this fourth preferred aspect preferably V is cyano or hydroxy.

Specific compounds of the present invention are:

2,3-dichloro-5-[N-(2-phenoxyethyl)carbamoyl]-4H-thieno[3,2-b]pyrrole;

2,3-dichloro-5-{N-[2-(2-thienyl)ethyl]carbamoyl}-4H-thieno[3,2-b]pyrrole;

2,3-dichloro-5-{N-[2-(2-methoxyphenyl)ethyl]carbamoyl}-4H-thieno[3,2-b]pyrrole;

2,3-dichloro-5-[N-(2-phenyl-1-cyclopropyl)carbamoyl]-4H-thieno[3,2-b]pyrrole;

2,3-dichloro-5-{N-[2-(4-fluorophenyl)ethyl]carbamoyl}-4H-thieno[3,2-b]pyrrole;

2,3-dichloro-5-[N-(N-phenylcarbamoylmethyl)carbamoyl]-4H-thieno[3,2-b]pyrrole;

2,3-dichloro-5-(N-{2-[(2-pyridyl)amino]ethyl}carbamoyl)-4H-thieno[3,2-b]pyrrole;

2,3-dichloro-5-{N-[2-(N-methylmethanesulphonamido)-1-(thiazol-2-yl)ethyl]carbamoyl}-4H-thieno[3,2-b]pyrrole;

2,3-dichloro-5-{N-[2-(thiomorpholino)ethyl]carbamoyl}-4H-thieno[3,2-b]pyrrole;

5-[N-(benzoylmethyl)carbamoyl]-2,3-dichloro-4H-thieno[3,2-b]pyrrole;

3-chloro-5-[N-(N-phenylcarbamoylmethyl)carbamoyl]-4H-thieno[3,2-b]pyrrole;

3-chloro-5-{N-[2-(thiomorpholino)ethyl]carbamoyl}-4H-thieno[3,2-b]pyrrole;

3-chloro-5-{N-[2-(N-methylmethanesulphonamido)-1-(thiazol-2-yl)ethyl]carbamioyl}-4H-thieno[3,2-b]pyrrole;

3-chloro-5-[N-(benzoylmethyl)carbamoyl]-4H-thieno[3,2-b]pyrrole;

3-chloro-5-{N-[2-(2-methoxyphenyl)ethyl]carbamoyl}-4H-thieno[3,2-b]pyrrole;

3-chloro-5-{N-[2-(2-thienyl)ethyl]carbamoyl}-4H-thieno[3,2-b]pyrrole;

3-chloro-5-[N-(2-phenyl-1-cyclopropyl)carbamoyl]-4H-thieno[3,2-b]pyrrole;

3-chloro-5-{N-[2-(4-fluorophenyl)ethyl]carbamoyl}-4H-thieno[3,2-b]pyrrole;

3-chloro-5-[N-(2-phenoxyethyl)carbamoyl]-4H-thieno[3,2-b]pyrrole;

3-chloro-5-{N-[2-(1-phenylmethanesulphonamido)ethyl]carbamoyl}-4H-thieno[3,2-b]pyrrole;

3-chloro-5-[N-(4-oxo-2,3,4,5-tetrahydrobenz[1,5]thiazepin-3-yl)carbamoyl]-4H-thieno[3,2-b]pyrrole;

2-chloro-5-[N-(benzoylmethyl)carbamoyl]-4H-thieno[3,2-b]pyrrole;

2-chloro-5-[N-(2-phenyl-1-cyclopropyl)carbamoyl]-4H-thieno[3,2-b]pyrrole;

2-chloro-5-[N-(N-phenylcarbamoylmethyl)carbanioyl]-4H-thieno[3,2-b]pyrrole;

2-chloro-5-(N-{2-[(2-pyridyl)amino]ethyl}carbamoyl)-4H-thieno[3,2-b]pyrrole;

2-chloro-5-{N-[2-(2-methoxyphenyl)ethyl]carbamoyl}-4H-thieno[3,2-b]pyrrole;

2-chloro-5-[N-(2-phenoxyethyl)carbamoyl]-4H-thieno[3,2-b]pyrrole;

2-chloro-5-{N-[2-(2-thienyl)ethyl]carbamoyl}-4H-thieno[3,2-b]pyrrole;

2-chloro-5-{N-[2-(4-fluorophenyl)ethyl]carbamoyl}-4H-thieno[3,2-b]pyrrole;

2-chloro-5-{N-[2-(N-methylmethanesulphonamido)-1-(thiazol-2-yl)ethyl]carbamoyl}-4H-thieno[3,2-b]pyrrole;

2-chloro-5-{N-[2-(thiomorpholino)ethyl]carbamoyl}-4H-thieno[3,2-b]pyrrole;

2,3-dichloro-5-[N-(2,3-dimethyl-5-oxo-1-phenyl-2,5-dihydro-1H-pyrazol-4-yl)carbamoyl]-4H-thieno[3,2-b]pyrrole;

2,3-dichloro-5-[N-(4-sulphamoylphenylmethyl)carbamoyl]-4H-thieno[3,2-b]pyrrole;

2,3-dichloro-5-[N-(2-hydroxy-1-phenethyl)carbamoyl]-4H-thieno[3,2-b]pyrrole;

2,3-dichloro-5-{N-(2-[(3-trifluoromethylpyrid-2-yl)amino]ethyl)carbamoyl}-4H-thieno[3,2-b]pyrrole;

2,3-dichloro-5-{N-[3-(5-tetrazolyl)propyl]carbamoyl}-4H-thieno[3,2-b]pyrrole;

2,3-dichloro-5-[N-(5-oxo-3-phenyl-4,5-dihydroisoxazol-4-yl)carbamoyl]-4H-thieno[3,2-b]pyrrole;

2,3-dichloro-5-[N-(5-hydroxy-2-oxo-2,3,4,5-tetrahydro-1H-benz[b]azepin-4-yl)carbamoyl]-4H-thieno[3,2-b]pyrrole;

2-chloro-5-{N-[3-(benzyloxycarbonylamino)propyl]carbamoyl}-4H-thieno[3,2-b]pyrrole;

2,3-dichloro-5-{N-[(4-dimethylaminophenyl)methyl]carbamoyl}-4H-thieno[3,2-b]pyrrole;

5-[N-(1-benzyl-2-hydroxyethyl)carbamoyl]-2,3-dichloro-4H-thieno[3,2-b]pyrrole;

2,3-dichloro-5-{N-[2-(phenylamino)ethyl]carbamoyl}-4H-thieno[3,2-b]pyrrole;

2,3-dichloro-5-[N-(β-(R)-hydroxy-α-methylphenethyl)carbamoyl]-4H-thieno[3,2-b]pyrrole;

2,3-dichloro-5-[N-(β-hydroxyphenethyl)carbamoyl)-4H-thieno[3,2-b]pyrrole;

2,3-dichloro-5-{N-[2-(4-hydroxyphenyl)ethyl]carbamoyl}4H-thieno[3,2-b]pyrrole;

2,3-dichloro-5-{N-[(benzimidazol-2-yl)methyl]carbamoyl}-4H-thieno[3,2-b]pyrrole;

2,3-dichloro-5-{N-[2-(4-chlorophenyl)-2-hydroxy-1-(methoxycarbonyl)ethyl]carbamoyl}-4H-thieno[3,2-b]pyrrole;

2,3-dichloro-5-{N-(imidazo[1,2-a]pyrid-2-yl)carbamoyl}-4H-thieno[3,2-b]pyrrole;

5-{N-[(benzthiazol-2-yl)methyl]carbamoyl}-2,3-dichloro-4H-thieno[3,2-b]pyrrole;

2,3-dichloro-5-{N-[(6-trifluoromethylpyrid-3-yl)methyl]carbamoyl}-4H-thieno[3,2-b]pyrrole;

2,3-dichloro-5-{N-(2-[(2-pyridazinyl)methyl]carbamioyl}-4H-thieno[3,2-b]pyrrole;

2,3-dichloro-5-{N-[N-(2-hydroxy-3-phenoxypropyl)carbamoylmethyl] carbamoyl}-4H-thieno[3,2-b]pyrrole;

2,3-dichloro-5-{N-[N-(3-methylisothiazol-5-yl)carbamoylmnethyl]carbamoyl}-4H-thieno[3,2-b]pyrrole;

2,3-dichloro-5-{N-[2-(pyridazin-3-yloxy)ethyl]carbamoyl}-4H-thieno[3,2-b]pyrrole;

2-chloro-5-(N-{2-[(3-trifluoromethylpyrid-2-yl)amino]ethyl}carbamoyl)-4H-thieno[3,2-b]pyrrole;

2,3-dichloro-5-{N-[2-(4-sulphamoylphenyl)ethyl]carbamoyl}-4H-thieno[3,2-b]pyrrole;

2,3-dichloro-5-{N-[2-(2-pyridyl)ethyl]carbamoyl}-4H-thieno[3,2-b]pyrrole;

2,3-dichloro-5-{N-(2-[1-hydroxymethyl-2-(4-imidazolyl)ethyl]carbamoyl}-4H-thieno[3,2-b]pyrrole;

2,3-dichloro-5-{N-(2-[(3-quinolyl)methyl]carbamoyl}-4H-thieno[3,2-b]pyrrole;

5-{N-[3-(4-acetamidophenoxy)-2-hydroxypropyl]carbamoyl}-2,3-dichloro-4H-thieno[3,2-b]pyrrole;

2,3-dichloro-5-{N-[3-(N-methylsulphonylcarbamoyl)propyl]carbamoyl}-4H-thieno[3,2-b]pyrrole;

2,3-dichloro-5-[N-(2-{[2-(guanidino)thiazol-4-yl]methylthio}ethyl)carbamoyl]-4H-thieno[3,2-b]pyrrole;

2,3-dichloro-5-{N-[2-(2,4-dioxoimidazolidin-1-yl)ethyl]carbamoyl}-4H-thieno[3,2-b]pyrrole;

5-{N-[2-benzylthio-1-(hydroxymethyl)ethyl]carbamoyl}-2,3-dichloro-4H-thieno[3,2-b]pyrrole;

2,3-dichloro-5-{N-[2-(dimethyaminosulphonylamino)ethyl]carbamoyl}-4H-thieno[3,2-b]pyrrole;

2,3-dichloro-5-{N-[(6-methoxypyrid-3-yl)niethyl]carbamoyl}-4H-thieno[3,2-b]pyrrole;

(S)-2,3-dichloro-5-{N-[(2-oxo-3-phenyl-2,3,4,5-tetrahydrooxazol-5-yl)methyl]carbamoyl}-4H-thieno[3,2-b]pyrrole;

2,3-dichloro-5-(N-{2-[3-(carbamoylmethyl)phenoxy]ethyl}carbamoyl)-4H-thieno[3,2-b]pyrrole;

5-(N-{[6-(benzo[1,3]dioxol-5-yl)-4-methylmorpholin-2-yl]methyl}carbamoyl)-2,3-dichloro-4H-thieno[3,2-b]pyrrole;

5-(N-benzylcarbamoyl)-2,3-dichloro-4H-thieno[3,2-b]pyrrole;

2,3-dichloro-5-(N-phenethylcarbamoyl)-4H-thieno[3,2-b]pyrrole;

2,3-dichloro-5-[N-(3-phenylpropyl)carbamoyl]-4H-thieno[3,2-b]pyrrole;

2,3-dichloro-5-{N-[2-(2-hydroxyphenyl)ethyl]carbamoyl}-4H-thieno[3,2-b]pyrrole;

2,3-dichloro-5-[N-(α,α-dimethylphenethyl)carbamoyl]-4H-thieno[3,2-b]pyrrole;

2,3-dichloro-5-[N-(1-phenylcyclobutyl)methyl)carbamoyl]-4H-thieno[3,2-b]pyrrole;

2,3-dichloro-5-[N-(β-methylphenethyl)carbamoyl]-4H-thieno[3,2-b]pyrrole;

2,3-dichloro-5-[N-(1,2,3,4-tetrahydronaphth-2-yl)carbamoyl]-4H-thieno[3,2-b]pyrrole;

5-[N-(N-benzylcarbamoylmethyl)carbamoyl]-2,3-dichloro-4H-thieno[3,2-b]pyrrole;

5-[N-(N-benzyl-N-methylcarbamoylmethyl)carbamoyl]-2,3-dichloro-4H-thieno[3,2-b]pyrrole;

2,3-dichloro-5-[N-(N-methyl-N-phenylcarbamoylmethyl)carbamoyl]-4H-thieno[3,2-b]pyrrole;

2,3-dichloro-5-{N-[N-(2-cyanoethyl)-N-phenylcarbamoylmethyl]carbamoyl}-4H-thieno[3,2-b]pyrrole;

2,3-dichloro-5-{N-[N-(4-methoxyphenyl)carbamoylmethyl]carbamoyl}-4H-thieno[3,2-b]pyrrole;

2,3-dichloro-5-{N-[N-(4-fluorophenyl)carbamoylmethyl]carbamoyl}-4H-thieno[3,2-b]pyrrole;

2,3-dichloro-5-{N-[N-(4-nitrophenyl)carbamoylmethyl]carbamoyl)-4H-thieno[3,2-b]pyrrole;

2,3-dichloro-5-{N-[N-(2,6-dimethylphenyl)carbamoylmethyl]carbamoyl}-4H-thieno[3,2-b]pyrrole;

2,3-dichloro-5-{N-[N-methyl-N-(4-methylphenyl)carbamoylmethyl]carbamoyl}-4H-thieno[3,2-b]pyrrole;

2,3-dichloro-5-{N-[N-methyl-N-(3-methylphenyl)carbamoylmethyl]carbamoyl}-4H-thieno[3,2-b]pyrrole;

2,3-dichloro-5-{N-[N-(3-chlorophenyl) N-methylcarbamoylmethyl]carbamoyl}-4H-thieno[3,2-b]pyrrole;

2,3-dichloro-5-{N-[N-(2-hydroxyethyl)-N-phenylcarbamoylmethyl]carbamoyl}-4H-thieno[3,2-b]pyrrole;

2,3-dichloro-5-{N-[N-(1,1-dimethyl-2-hydroxyethyl)carbamoylmnethyl]carbamoyl}-4H-thieno[3,2-b]pyrrole;

2,3-dichloro-5-{N-[N-(2-hydroxyethyl)-N-methylcarbamoylmethyl]carbamoyl}-4H-thieno[3,2-b]pyrrole;

2,3-dichloro-5-{N-[N-(2-hydroxyethyl)carbamoylmethyl]carbamoyl}-4H-thieno[3,2-b]pyrrole;

2,3-dichloro-5-{N-[N-(3-hydroxypropyl)carbamoylmethyl]carbanioyl}-4H-thieno[3,2-b]pyrrole;

2,3-dichloro-5-{N-[N-(4-hydroxybutyl)carbamoylmethyl]carbamoyl}-4H-thieno[3,2-b]pyrrole;

2,3-dichloro-5-(N-{N-[bis(hydroxymethyl)methyl]carbamoylmethyl}carbamoyl)-4H-thieno[3,2-b]pyrrole;

2,3-dichloro-5-{N-[N-(2,3-dihydroxypropyl)carbamoylmethyl]carbamoyl}-4H-thieno[3,2-b]pyrrole;

2,3-dichloro-5-{N-[N-(4-hydroxymethylphenyl)carbamoylmethyl]carbamoyl}-4H-thieno[3,2-b]pyrrole;

2,3-dichloro-5-{N-[N-(5-isoquinolyl)carbamoylmethyl]carbamoyl}-4H-thieno[3,2-b]pyrrole;

2,3-dichloro-5-{N-[N-(3-hydroxymethy)lphenyl]carbamoylmethyl]carbamoyl}-4H-thieno[3,2-b]pyrrole;

2,3-dichloro-5-(N-{N-[4-(2-hydroxyethyl)phenyl]carbamoylmethyl}carbamoyl)-4H-thieno[3,2-b]pyrrole;

2,3-dichloro-5-{N-[N-(2,4-difluorophenyl)-N-methyl-carbamoylmethyl]carbamoyl}-4H-thieno[3,2-b]pyrrole;

2,3-dichloro-5-{N-[(1,2,3,4-tetrahydro-1-quinolyl)carbonylmethyl]carbamoyl}-4H-thieno[3,2-b]pyrrole;

2,3-dichloro-5-{N-[N-(2-cyanoethyl)-N-methylcarbamoylmethyl]carbamoyl}-4H-thieno[3,2-b]pyrrole;

2,3-dichloro-5-{N-[N-(4-hydroxypiperidino)carbamoylmethyl]carbamoyl}-4H-thieno[3,2-b]pyrrole;

2,3-dichloro-5-[N-(N-cyclopentylcarbamoylmethyl)carbamoyl]-4H-thieno[3,2-b]pyrrole;

2,3-dichloro-5-[N-(N-isopropylcarbamoylmethyl)carbaxnoyl]-4H-thieno[3,2-b]pyrrole;

2,3-dichloro-5-[N-(N-isopropyl-N-methylcarbamoylmethyl)carbamoyl]-4H-thieno[3,2-b]pyrrole;

2,3-dichloro-5-[N-(thiomorpholinocarbonylmethyl)carbamoyl]-4H-thieno[3,2-b]pyrrole;

2,3-dichloro-5-[N-(morpholinocarbonylmethyl)carbamoyl]-4H-thieno[3,2-b]pyrrole;

2,3-dichloro-5-{N-[(1,1-dioxothiomorpholino)carbonylmethyl]carbamoyl}-4H-thieno[3,2b]-pyrrole;

2,3-dichloro-5-{N-[(1-oxothiomorpholino)carbonylmethyl]carbamoyl}-4H-thieno[3,2-b]pyrrole;

2-chloro-5-[N-(2-indanyl)carbamoyl]-6H-thieno[2,3-b]pyrrole;

5-[N-(benz[1,2]oxazol-3-ylmethyl)carbamoyl]-2,3-dichloro-4H-thieno[3,2-b]pyrrole;

2,3-dichloro-5-(N-{2-[2-(hydroxymethyl)phenyl]ethyl}carbamoyl)-4H-thieno[3,2-b]pyrrole;

2,3-dichloro-5-[N-(4-phenylisoxazol-3-ylmethyl)carbamoyl]-4H-thieno[3,2-b]pyrrole;

2,3-dichloro-5-(N-{2-[2-(2-morpholinoethoxy)phenyl]ethyl}carbamoyl)-4H-thieno[3,2-b]pyrrole;

2,3-dichloro-5-(N-{2-[2-(methoxycarbonylmethoxy)phenyl]ethyl}carbamoyl)-4H-thieno[3,2-b]pyrrole;

5-(N-{2-[2-(carboxymethoxy)phenyl]ethyl}carbamoyl)-2,3-dichloro-4H-thieno[3,2-b]pyrrole;

2,3-dichloro-5-{N-[2-(3-methoxyphenyl)ethyl]carbamoyl}-4H-thieno[3,2-b]pyrrole;

2,3-dichloro-5-[N-(2-oxo-1,2,3,4-tetrahydroquinol-3-yl)carbamoyl]-4H-thieno[3,2-b]pyrrole;

2,3-dichloro-5-(N-{2-[2-(2-methoxyethoxy)phenyl]ethyl}carbamoyl)-4H-thieno[3,2-b]pyrrole;

5-(N-{2-[2-(carbamoylmethoxy)phenyl]ethyl}carbamoyl)-2,3-dichloro-4H-thieno[3,2-b]pyrrole;

2,3-dichloro-5-(N-{2-[2-(N-methylcarbamoylmethoxy)phenyl]ethyl}carbamoyl)-4H-thieno[3,2-b]pyrrole;

2,3-dichloro-5-(N-{2-[2-(N,N-dimethylcarbamoylmethoxy)phenyl]ethyl}carbamoyl)-4H-thieno[3,2-b]pyrrole;

2,3-dichloro-5-(N-{2-[2-(morpholinocarbonylmethoxy)phenyl]ethyl}carbamoyl)-4H-thieno[3,2-b]pyrrole;

5-(N-{2-[2-(N-benzylcarbamoylmethoxy)phenyl]ethyl}carbamoyl)-2,3-dichloro-4H-thieno[3,2-b]pyrrole;

2,3-dichloro-5-(N-{2-[2-(4-hydroxypiperidinocarbonylmethoxy)phenyl]ethyl}carbamoyl)-4H-thieno[3,2-b]pyrrole;

(S)-2-chloro-5-{N-[α-(5-ethoxycarbonyl-1,3,4-oxadiazol-2-yl)phenethyl]carbamoyl}-6H-thieno[2,3-b]pyrrole;

(S)-2-chloro-5-{N-[α-(4-methoxycarbonyloxazol-5-yl)phenethyl]carbamoyl}-6H-thieno[2,3-b]pyrrole;

2-chloro-5-{N-[α-(3-pyridyl)phenethyl]carbamoyl}-6H-thieno[2,3-b]pyrrole;

2,3-dichloro-5-{N-[α-(3-pyridyl)phenethyl)]carbamoyl}-4H-thieno[3,2-b]pyrrole;

(S)-2-chloro-5-{N-[α-(3-phenyl-1,2,4-oxadiazol-5-yl)phenethyl]carbamoyl}-6H-thieno[2,3-b]pyrrole;

2,3-dichloro-5-[N-(1-hydroxyindan-2-yl)carbamoyl]-4H-thieno[3,2-b]pyrrole;

2,3-dichloro-5-[N-((1S, 2S)-1-hydroxyindan-2-yl)carbamoyl]-4H-thieno[3,2-b]pyrrole;

2,3-dichloro-5-[N-((1R, 2R)-1-hydroxyindan-2-yl)carbamoyl]-4H-thieno[3,2-b]pyrrole;

2-chloro-5-[N-(1-hydroxyindan-2-yl)carbamoyl]-6H-thieno[2,3-b]pyrrole;

2,3-dichloro-5-[N-(1-hydroxy-1,2,3,4-tetrahydronaphth-2-yl)carbanioyl]-4H-thieno[3,2-b]pyrrole;

2,3-dichloro-5-[N-(6-fluoro-1-hydroxyindan-2-yl)carbamoyl]-4H-thieno[3,2-b]pyrrole;

2,3-dichloro-5-[N-(7-methoxy-1-oxo-1,2,3,4-tetrahydronaphth-2-yl)carbamoyl]-4H-thieno[3,2-b]pyrrole;

2,3-dichloro-5-[N-(2-indanyl)carbamoyl]-4H-thieno[3,2-b]pyrrole;

2,3-dichloro-5-[N-(3-methylisoxazol-5-yl)methylcarbamoyl]-4H-thieno[3,2-b]pyrrole;

2,3-dichloro-5-[N-(4-hydroxy-1,1-dioxotetrahydrothiophen-3-yl)carbamoyl]-4H-thieno[3,2-b]pyrrole;

2,3-dichloro-5-(N-{N-methyl-N-[(1-oxo-1,2,3,4-tetrahydronaphth-2-yl)mnethyl]carbamoylmethyl}carbamoyl)-4H-thieno[3,2-b]pyrrole;

2-chloro-5-[N-(2-oxo-1,2,3,4-tetrahydroquinol-3-yl)carbamoyl]-6H-thieno[2,3-b]pyrrole;

2-chloro-5-[N-(1,2,3,4-tetrahydroquinol-3-yl)carbamoyl]-6H-thieno[2,3-b]pyrrole;

2-chloro-5-[N-(1-methyl-2-oxo-1,2,3,4-tetrahydroquinol-3-yl)carbamoyl]-6H-thieno[2,3-b]pyrrole;

2-chloro-5-[N-(3-oxo-2,3,4,5-tetrahydro-1H-benz[2]azepin-4-yl)carbamoyl]-6H-thieno[2,3-b]pyrrole;

2,3-dichloro-5-[N-(1-methoxyindan-2-yl)carbamoyl]-4H-thieno[3,2-b]pyrrole;

2,3-dichloro-5-(N-{1-[N-(1,1-dimethylethoxy)carbonylamino]indan-2-yl}carbamoyl)-4H-thieno[3,2-b]pyrrole;

5-[N-(1-aminoindan-2-yl)carbamoyl]-2,3-dichloro-4H-thieno[3,2-b]pyrrole;

5-[N-(1-acetamidoindan-2-yl)carbamoyl]-2,3-dichloro-4H-thieno[3,2-b]pyrrole;

2,3-dichloro-5-{N-[1-(methanesulphonamido)indan-2-yl]carbamoyl}-4H-thieno[3,2-b]pyrrole;

2,3-dichloro-5-{N-[1-(methylamino)indan-2-yl]carbamoyl}-4H-thieno[3,2-b]pyrrole; and

2,3-dichloro-5-{N-[1-(N-methylacetamido)indan-2-yl]carbamoyl}-4H-thieno[3,2-b]pyrrole.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt or an in vivo hydrolysable ester thereof which process (wherein R¹, R², R³, —X-Y-Z-and n are, unless otherwise specified, as defined in formula (I)) comprises of:

a) reacting an acid of the formula (II):

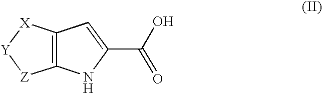

or an activated derivative thereof; with an amine of formula (III):

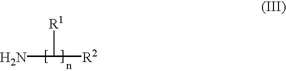

and thereafter if necessary:

i) converting a compound of the formula (I) into another compound of the formula (I);

ii) removing any protecting groups;

iii) forming a pharmaceutically acceptable salt or in vivo hydrolysable ester.

Specific reaction conditions for the above reaction are as follows.

Process a) Acids of formula (II) and amines of formula (III) may be coupled together in the presence of a suitable coupling reagent. Standard peptide coupling reagents known in the art can be employed as suitable coupling reagents, or for example carbonyldiimidazole, 1-ethyl-3-(3-dimethylaminopropyl)carbodi-imide hydrochloride and dicyclohexyl-carbodiimide, optionally in the presence of a catalyst such as 1-hydroxybenzotriazole, dimethylaminopyridine or 4-pyrrolidinopyridine, optionally in the presence of a base for example triethylamine, di-isopropylethylamine, pyridine, or 2,6-di-alyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

The acids of formula (II) may be prepared according to Scheme 1:

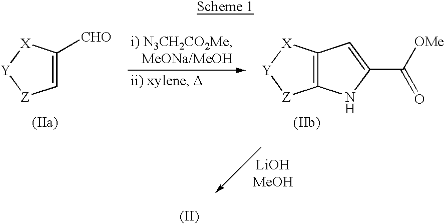

Compounds of formula (IIa) and amines of formula (III) are commercially available or they are known compounds or they are prepared by processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses glycogen phosphorylase inhibitory activity. This property may be assessed, for example, using the procedure set out below.

Assay

The activity of the compounds is determined by measuring the inhibitory effect of the compounds in the direction of glycogen synthesis, the conversion of glucose-1-phosphate into glycogen with the release of inorganic phosphate, as described in EP 0 846 464 A2. The reactions were in 96well microplate format in a volume of 100 μl. The change in optical density due to inorganic phosphate formation was measured at 620 nM in a Labsystems iEMS Reader MF by the general method of (Nordlie R. C and Arion W. J, Methods of Enzymology, 1966, 619-625). The reaction is in 50 mM HEPES, 2.5 nM MgCl₂, 2.25 mM ethylene glycol-bis(b-aminoethyl ether) N,N,N′,N′-tetraacetic acid, 100 mM KCl, 2 mM D-(+)-glucose pH 7.2, containing 0.5 mM dithiothreitol, the assay buffer solution, with 0.1 mg type m glycogen, 0.15 ug glycogen phosphorylase a (GPa) from rabbit muscle and 0.5 mM glucose-1-phosphate. GPa is pre-incubated in the assay buffer solution with the type m glycogen at 2.5 mg ml for 30 minutes. 40 μl of the enzyme solution is added to 25 μl assay buffer solution and the reaction started with the addition of 25 μl 2 mM glucose-1-phosphate. Compounds to be tested are prepared in 10 μl 10% DMSO in assay buffer solution, with final concentration of 1% DMSO in the assay. The non-inhibited activity of GPa is measured in the presence of 10 μl 10% DMSO in assay buffer solution and maximum inhibition measured in the presence of 30 μM CP320626 (Hoover et al (1998) J Med Chem 41, 2934-8; Martin et al (1998) PNAS 95, 1776-81). The reaction is stopped after 30 min with the addition of 50 μl acidic ammonium molybdate solution, 12 ug ml⁻¹ in 3.48% H₂SO₄ with 1% sodium lauryl sulphate and 10 ug ml⁻¹ ascorbic acid. After 30 minutes at room temperature the absorbency at 620 nm is measured.

The assay is performed at a test concentration of inhibitor of 10 μM or 100 μM. Compounds demonstrating significant inhibition at one or both of these concentrations may be further evaluated using a range of test concentrations of inhibitor to determine an IC₅₀, a concentration predicted to inhibit the enzyme reaction by 50%.

Activity is calculated as follows:—

% inhibition=(1−(compound OD620−fully inhibited OD620)/(non-inhibited rate OD620−fully inhibited OD620))*100.

OD620=optical density at 620 nM.

Typical IC₅₀ values for compounds of the invention when tested in the above assay are in the range 100 μM to 1 nM.

The activity of the compounds is alternatively determined by measuring the inhibitory effect of the compounds on glycogen degradation, the production of glucose-1-phosphate from glycogen is monitored by the multienzyme coupled assay, as described in EP 0 846 464 A2, general method of Pesce et al (Pesce, M A, Bodourian, S H, Harris, R C, and Nicholson, J F (1977) Clinical Chemistry 23, 1171-1717). The reactions were in 384well microplate format in a volume of 50 μl. The change in fluorescence due to the conversion of the co-factor NAD to NADH is measured at 340 nM excitation, 465 nm emission in a Tecan Ultra Multifunctional Microplate Reader. The reaction is in 50 mM HEPES, 3.5 mM KH₂PO₄, 2.5 mM MgCl₂, 2.5 mM ethylene glycol-bis(b-aminoethyl ether) N,N,N′,N′-tetraacetic acid, 100 mM KCl, 8 mM D-(+)-glucose pH 7.2, containing 0.5 mM dithiothreitol, the assay buffer solution. Human recombinant liver glycogen phosphorylase a (hrl GPa) 20 nM is pre-incubated in assay buffer solution with 6.25 mM NAD, 1.25 mg type III glycogen at 1.25 mg ml⁻¹ the reagent buffer, for 30 minutes. The coupling enzymes, phosphoglucomutase and glucose-6-phosphate dehydrogenase (Sigma) are prepared in reagent buffer, final concentration 0.25Units per well. 20 μl of the hrl GPa solution is added to 10 μl compound solution and the reaction started with the addition of 20 ul coupling enzyme solution. Compounds to be tested are prepared in 10 μl 5% DMSO in assay buffer solution, with final concentration of 1% DMSO in the assay. The non-inhibited activity of GPa is measured in the presence of 10 μl 5% DMSO in assay buffer solution and maximum inhibition measured in the presence of 5 mgs ml⁻¹ N-ethylmaleimide. After 6 hours at 30° C. Relative Fluoresence Units (RFUs) are measured at 340 nM excitation, 465 nm emission.

The assay is performed at a test concentration of inhibitor of 10 μM or 100 μM. Compounds demonstrating significant inhibition at one or both of these concentrations may be further evaluated using a range of test concentrations of inhibitor to determine an IC₅₀, a concentration predicted to inhibit the enzyme reaction by 50%.

Activity is calculated as follows:—

% inhibition=(1−(compound RFUs−fully inhibited RFUs)/(non-inhibited rate RFUs−fully inhibited RFUs))*100.

Typical IC₅₀ values for compounds of the invention when tested in the above assay are in the range 100 μM to 1 nM.

The inhibitory activity of compounds was further tested in rat primary hepatocytes. Rat hepatocytes were isolated by the collagenase perfusion technique, general method of Seglen (P. O. Seglen, Methods Cell Biology (1976) 13 29-83). Cells were cultured on Nunclon six well culture plates in DMEM with high level of glucose containing 10% foetal calf serum, NEAA, Glutamine, penicillin/streptomycin ((100 units/100 ug)/ml) for 4 to 6 hours. The hepatocytes were then cultured in the DMEM solution without foetal calf serum and with 10 nM insulin and 10 nM dexamethasone. Experiments were initiated after 18-20 hours culture by washing the cells and adding Krebs-Henseleit bicarbonate buffer containing 2.5 mM CaCl₂ and 1% gelatin. The test compound was added and 5 minutes later the cells were challenged with 25 nM glucagon. The Krebs-Henseleit solution was removed after 60 min incubation at 37° C., 95%O₂/5%CO₂ and the glucose concentration of the Krebs-Henseleit solution measured.

According to a further aspect of the invention there is provided a pharmaceutical composition which comprises a compound of the formula (I), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg per square meter body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt or in vivo hydrolysable ester thereof, as defined hereinbefore, for use in a method of treatment of a warm-blooded animal such as man by therapy.

According to an additional aspect of the invention there is provided a compound of the formula (I), or a pharmaceutically acceptable salt or in vivo hydrolysable ester thereof, as defined hereinbefore, for use as a medicament.

According to an another aspect of the invention there is provided the use of a compound of the formula (I′):

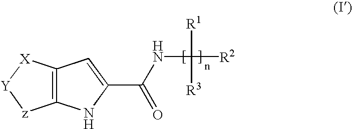

wherein:

—X-Y-Z- is selected from —S—CR⁴═CR⁵—, —CR⁴═CR⁵—S—, —O—CR⁴═CR⁵—, —CR⁴═CR⁵—O—, —N═CR⁴—S—, —S—CR⁴═N—, —NR⁶—CR⁴═CR⁵— and —CR⁴═CR⁵—NR⁶—;

wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;

R⁶ is hydrogen or C₁₋₆alkyl;

R¹ is selected from hydrogen, halo, nitro, cyano, hydroxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl) amino, N,N-(C₁₋₆alkyl)₂amino, C₁₋₆alkanoylamino, N-(C₁₋₆alkyl)carbamoyl, N,N-(C₁₋₄alkyl)₂carbamoyl, C₁₋₆alkylS(O), wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, C₃₋₈cycloalkylC₁₋₆alkyl, aryl, arylC₁₋₆alkyl, heterocyclic group and (heterocyclic group)C₁₋₆alkyl; wherein R¹ may be optionally substituted on carbon by one or more groups selected from P and wherein if said heterocyclic group contains an —NH— moiety that nitrogen may be optionally substituted by a group selected from R;

R² is selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₆alkanoylamino, N-(C₁₋₆alkyl)carbamoyl, N,N-(C₁₋₄alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, sulphamoylamino, N-(C₁₋₆alkyl)sulphamoylamino, N,N-(C₁₋₆alkyl)₂sulphamoylamino, C₁₋₆alkylsulphonylamino, C₁₋₆alkylsulphonylaminocarbonyl, C₁₋₆alkylsulphonyl-N-(C₁₋₆alkyl)amino and a group -E-F-G-H;

wherein E and G are independently selected from a direct bond, —O—, —S—, —SO—, —SO₂—, —OC(O)—, —C(O)O—, —C(O)—, —NR^{a}—, —NR^{a}C(O)—, —C(O)NR^{a}—, —SO₂NR^{a}—, —NR^{a}SO₂—, —NR^{a}C(O)NR^{b}—, —OC(O)NR^{a}—, —NR^{a}C(O)O—, —NR^{a}SO₂NR^{b}—, —SO₂NR^{a}C(O)— and —C(O)NR^{a}SO₂—; wherein R^{a} and R^{b} are independently selected from hydrogen or C₁₋₆alkyl which is optionally substituted by a group V;

F is C₁₋₆alkylene optionally substituted by one or more Q or a direct bond;

H is selected from aryl, C₃₋₈cycloalkyl and heterocyclic group; wherein H may be optionally substituted on carbon by one or more groups selected from S and wherein if said heterocyclic group contains an —NH— moiety that nitrogen may be optionally substituted by a group selected from T;

R³ is hydrogen or C₁₋₆alkyl;

n is selected from 0-4; wherein the values of R¹ may be the same or different; and wherein the values of R³ may be the same or different;

P, S and Q are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein P, S and Q may be optionally and independently substituted on carbon by one or more groups selected from V and wherein if said heterocyclic group contains an —NH— moiety that nitrogen may be optionally substituted by a group selected from U;

V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl, and 4-hydroxypiperidinocarbonyl;

R, T and U are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)carbamoyl, phenyl, benzyl, benzyloxycarbonyl, benzoyl and phenylsulphonyl; or a pharmaceutically acceptable salt or an in vivo hydrolysable ester thereof;

with the proviso that

when —X-Y-Z- is as initially defined, n is 1, R¹ is arylmethyl, substituted arylmethyl, (heterocyclic group)methyl and substituted (heterocyclic group)methyl and R³ is hydrogen then R² is not a group —C(═O)-A or a group —CH(OH)—C(═O)-A in which A is NR^{d}R^{d}, —NR^{a}CH₂CH₂OR^{a}, or

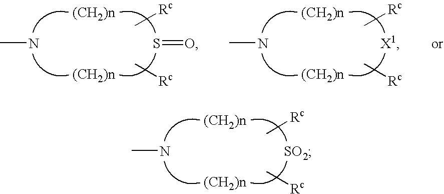

each R^{a} and R^{b} is independently hydrogen or —C₁-C₈alkyl;

each R^{d} is independently hydrogen, C₁-C₈alkyl, C₁-C₈alkoxy, aryl, substituted aryl, heteroaryl, or substituted heteroaryl;

each R^{c} is independently hydrogen, —C(═O)OR^{a}, —OR^{a}, —SR^{a}, or —NR^{a}R^{a}; and each n is independently 1-3, and

X¹ is NR^{a}, —CH₂—, O or S;

or a pharmaceutically acceptable salt or in vivo hydrolysable ester thereof, as defined hereinbefore in the manufacture of a medicament for use in the production of a glycogen phosphorylase inhibitory effect in a warm-blooded animal such as man.

According to this another aspect of the invention there is provided the use of a compound of the formula (I′), or a pharmaceutically acceptable salt or in vivo hydrolysable ester thereof, as defined hereinbefore in the manufacture of a medicament for use in the treatment of type 2 diabetes, insulin resistance, syndrome X, hyperinsulinaemia, hyperglucagonaemia, cardiac ischaemia or obesity in a warm-blooded animal such as man.

According to this another aspect of the invention there is provided the use of a compound of the formula (I′), or a pharmaceutically acceptable salt or in vivo hydrolysable ester thereof, as defined hereinbefore in the manufacture of a medicament for use in the treatment of type 2 diabetes in a warm-blooded animal such as man.

According to a further feature of this aspect of the invention there is provided a method of producing a glycogen phosphorylase inhibitory effect in a warm-blooded animal, such as man, in need of such treatment which comprises administering to said animal an effective amount of a compound of formula (I′).

According to this further feature of this aspect of the invention there is provided a method of treating type 2 diabetes, insulin resistance, syndrome X, hyperinsulinaemia, hyperglucagonaemia, cardiac ischaemia or obesity in a warm-blooded animal, such as man, in need of such treatment which comprises administering to said animal an effective amount of a compound of formula (I′).

According to this further feature of this aspect of the invention there is provided a method of treating type 2 diabetes in a warm-blooded animal, such as man, in need of such treatment which comprises administering to said animal an effective amount of a compound of formula (I′).

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C. and under an atmosphere of an inert gas such as argon;

(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;

(iii) chromatography means flash chromatography on silica gel; thin layer chromatography (TLC) was carried out on silica gel plates; where a Bond Elut column is referred to, this means a column containing 10 g or 20 g or 50 g of silica of 40 micron particle size, the silica being contained in a 60 ml disposable syringe and supported by a porous disc, obtained from Varian, Harbor City, Calif., USA under the name “Mega Bond Elut SI”; “Mega Bond Elut” is a trademark; where a Biotage cartridge is referred to this means a cartridge containing KP-SIL™ silica, 60 angstroms, particle size 32-63 mM, supplied by Biotage, a division of Dyax Corp., 1500 Avon Street Extended, Charlottesville, Va. 22902, USA;

(iv) in general, the course of reactions was followed by TLC and reaction times are given for illustration only;

(v) yields are given for illustration only and are not necessarily those which can be obtained by diligent process development; preparations were repeated if more material was required;

(vi) where given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-δ₆) as solvent unless otherwise indicated, other solvents (where indicated in the text) include deuterated chloroform CDCl₃;

(vii) chemical symbols have their usual meanings; SI units and symbols are used;

(viii) reduced pressures are given as absolute pressures in Pascals (Pa); elevated pressures are given as gauge pressures in bars;

(ix) solvent ratios are given in volume: volume (v/v) terms;

(x) mass spectra (MS) were run with an electron energy of 70 electron volts in the chemical ionisation (CI) mode using a direct exposure probe; where indicated ionisation was effected by electron impact (EI), fast atom bombardment (FAB) or electrospray (ESP); values for m/z are given; generally, only ions which indicate the parent mass are reported and unless otherwise stated the value quoted is (M−H)⁻;

(xi) The following abbreviations are used:

| | |
|---|---|
| SM | starting material; |
| EtOAc | ethyl acetate; |
| MeOH | methanol; |
| DCM | dichloromethane |
| HOBT | 1-hydroxybenzotriazole |
| DIAD | diisopropyl azodicarboxylate |
| HATU | O-(7-azabenzotriazol-1-yl)-N, N, N′, N′, tetramethyluronium |
| | hexafluorophosphate |
| TFA | trifluoroacetic acid |
| DIPEA | di-isopropylethylamine; and |
| EDAC | 1-ethyl-3-(3-dimethylaminopropyl)carbodi-imide |
| | hydrochloride |

### Example #1

2,3-Dichloro-5-[N-(2-phenoxyethyl)carbamoyl]-4H-thieno[3,2-b]pyrrole

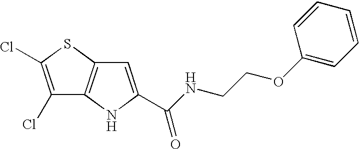

5-Carboxy-2,3-dichloro-4H-thieno[3,2-b]pyrrole (Method #9; 47 mg, 0.2 mmol) was dissolved in DCM (10 ml) containing 2-phenoxyethylamine (27 mg, 0.2 mmol), HOBT (27 mg, 0.2 mmol) and DIPEA (70 ml, 0.4 mmol). The mixture was stirred for 1 minute before the addition of EDAC (50 mg, 0.26 mmol). The mixture was stirred at ambient temperature for approximately 18 hours before being washed with water. The organic fraction was concentrated and was purified on a Bond Elut column (eluent 1:1 EtOAc/isohexane) to afford the title compound as an off white solid (32 mg). NMR: 12.4 (1H, br), 8.4 (1H, t), 7.3 (1H, d), 7.1 (1H, s), 6.9 (2H, m), 3.5 (2H, m), 3.0 (2H, t); m/z 353.2.

### Examples #2-#9

The following compounds were made by the process of Example #1 using 5-carboxy-2,3-dichloro-4H-thieno[3,2-b]pyrrole (Method #9) and the appropriate amine:

### Example #2

### 2,3-Dichloro-5-{N-[2-(2-thienyl)ethyl]carbamoyl}-4H-thieno[3,2-b]pyrrole

### Example #3

### 2,3-Dichloro-5-{N-[2-(2-methoxyphenyl)ethyl]carbamoyl}-4H-thieno[3,2-b]pyrrole

### Example #4

### 2,3-Dichloro-5-[N-(2-phenyl-1-cyclopropyl)carbamoyl]-4H-thieno[3,2-b]pyrrole

### Example #5

### 2,3-Dichloro-5-{N-[2-(4-fluorophenyl)ethyl]carbamoyl}-4H-thieno[3,2-b]pyrrole

### Example #6

### 2,3-dichloro-5-[N-(AT-phenylcarbamoylmethyl)carbamoyl]-4H-thieno[3,2-b]pyrrole

### Example #7

### 2,3-Dichloro-5-(N-{2-[(2-pyridyl)amino]ethyl}carbamoyl)-4H-thieno[3,2-b]pyrrole

### Example #8

### 2,3-Dichloro-5-{N-[2-(N-methylmethanesulphonamido)-1-(thiazol-2-yl)ethyl]carbamoyl}-4H-thieno[3,2-b]pyrrole

### Example #9

### 2,3-Dichloro-5-{N-[2-(thiomorpholino)ethyl]carbamoyl}-4H-thieno[3,2-b]pyrrole

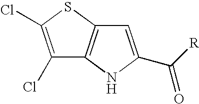

| Ex | R | NMR | M/z |
|---|---|---|---|
| #2 | 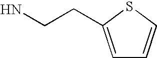 | 12.4 (1H, br), 8.4 (1H, t), 7.3 (1H, d), 7.1 (1H, s), 6.9 (2H, m), 3.5 (2H, m), 3.0 (2H, t) | 343.1 |
| #3 | 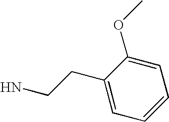 | 12.3 (1H, s), 8.3 (1H, t), 6.8-7.2 (5H, m), 3.8 (3H, s), 3.4 (2H, m), 2.8 (2H, t) | 367.3 |
| #4 | 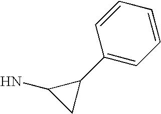 | 12.4 (1H, s), 8.5 (1H, d), 7.2 (5H, m), 7.1 (1H, s), 3.0 (1H, m), 2.1 (1H, m), 1.3 (2H, m) | 349.3 |
| #5 | 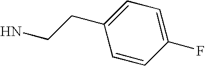 | 12.3 (1H, s), 8.4 (1H, t), 7.3 (2H, m), 7.1 (3H, m), 3.4 (2H, m), 2.8 (2H, t) | 355.3 |
| #6¹ | 12.4 (1H, s), 10.0 (1H, s), 8.7 (1H, br), 7.1-7.6 (5H, m), 7.2 (1H, s), 4.1 (2H, d) | 366.2 | |
| #7^{1,2} | 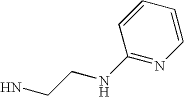 | 12.4 (1H, s), 8.4 (1H, s), 6.5-8.0 (5H, m), 6.6 (1H, s), 3.3 (4H, m) | 353.3 |
| #8³ | 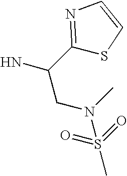 | (CDCl₃) 9.6 (1H, br), 7.8 (1H, m), 7.7 (1H, m), 7.3 (1H, m), 7.0 (1H, s), 5.6 (1H, m), 3.9 (1H, m), 3.7 (1H, m), 2.9 (3H, s), 2.8 (3H, s) | 451.2 |
| #9 | 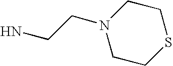 | (CDCl₃) 9.8 (1H, br), 6.9 (1H, br), 6.7 (1H, s), 4.1 (2H, m), 3.6 (2H, m), 2.8 (8H, m) | 36.2 |

¹Water was added and the title compound precipitated as a yellow solid which was filtered off, washed with water, dried under reduced pressure and was not purified further.
²Amine: Eur J Med Chem, 1987, 22, 91.
³Amine: Method #15

### Example #10

### 5-[N-(Benzoylmethyl)carbamoyl]-2,3-dichloro-4H-thieno[3,2-b]pyrrole

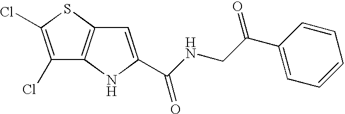

2,3-Dichloro-5-{N-[(2-phenyl-1,3-dioxolan-2-yl)methyl]carbamoyl}-4H-thieno[3,2-b]pyrrole (Method #17; 80 mg, 0.2 mmol) was dissolved in acetone (15 ml) containing aqueous hydrochloric acid (2.0 M, 1 ml). The mixture was heated under reflux for 90 minutes. The white precipitate formed was filtered off and washed with acetone. The filtrate was concentrated and the residue was triturated with water. The solid formed was filtered off and dried under reduced pressure to afford the title compound as a white solid (20 may). NMR: 12.4 (1H, s), 8.6 (1H, t), 7.6-8.0 (5H, m), 7.2 (1H, s), 4.8 (2H, d); m/z 351.1.

### Example #11

### 3-Chloro-5-[N-(N-phenylcarbamoylmethyl)carbamoyl]-4H-thieno[3,2-b]pyrrole

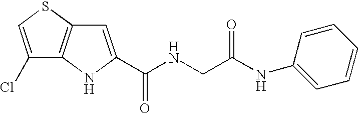

5-Carboxy-3-chloro-4H-thieno[3,2-b]pyrrole (Method #7; 100 mg, 0.5 mmol) was dissolved in DCM (6 ml) containing HOBT (68 mg, 0.5 mmol), DIPEA (176 ml, 1.0 mmol) and N-glycylaniline (75 mg, 0.5 mmol). The mixture was allowed to stand for one minute before the addition of EDAC (150 mg, 0.7 mmol). The solution was allowed to stand for approximately 18 hours before being washed with water. The organic phase was dried, filtered and concentrated under reduced pressure to afford the title compound (112 mg, 67%). NMR (CDCl₃) 9.7 (1H, br), 8.6 (1H, br), 7.0-8.0 (8H, m), 4.3 (2H, d); m/z 332.1.

### Examples #12-#21

The following compounds were made by the process of Example #11 using 5-carboxy-3-chloro-4H-thieno[3,2-b]pyrrole (Method #7) and the appropriate amine:

### Example #12

### 3-Chloro-5-{N-[2-(thiomorpholino)ethyl]carbamoyl}-4H-thieno[32-b]pyrrole

### Example #13

### 3-Chloro-5-{N-[2-(N-methylmethanesulphonamido)-1-(thiazol-2-yl)ethyl]carbamoyl}-4H-thieno[3,2-b]pyrrole

### Example #14

### 3-Chloro-5-[N-(benzoylmethyl)carbamoyl]-4H-thieno[3,2-b]pyrrole

### Example #15

### 3-Chloro-5-{N-[2-(2-methoxyphenyl)ethyl]carbamoyl}-4H-thieno[3,2-b]pyrrole

### Example #16

### 3-Chloro-5-{N-[2-(2-thienyl)ethyl]carbamoyl}-4H-thieno[3,2-b pyrrole

### Example #17

### 3-Chloro-5-[N-(2-phenyl-1-cyclopropyl)carbamoyl]-4H-thieno[3,2-b]pyrrole

### Example #18

### 3-Chloro-5-{N-[2-(4-fluorophenyl)ethyl]carbamoyl}-4H-thieno[3,2-b]pyrrole

### Example #19

### 3-Chloro-5-[N-(2-phenoxyethyl)carbamoyl]-4H-thieno[3,2-b]pyrrole

### Example #20

### 3-Chloro-5-{N-[2-(1-phenylmethanesulphonamido)ethyl]carbamoyl}-4H-thieno[3,2-b]pyrrole

### Example #21

### 3-Chloro-5-[N-(4-oxo-2,3,4,5-tetrahydrobenz[1,5]thiazepin-3-yl)carbamoyl]-4H-thieno[3,2-b]pyrrole

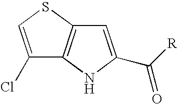

| Ex | R | NMR | M/z |
|---|---|---|---|
| #12 | 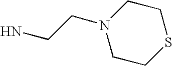 | (CDCl₃) 9.6 (1H, br), 7.0 (1H, s), 6.7 (1H, s), 6.6 (1H, br), 4.1 (2H, m), 3.5 (2H, m), 2.7 (8H, m) | 328.3 |
| #13 | 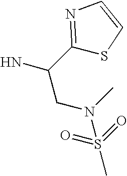 | (CDCl₃) 9.5 (1H, br), 7.8 (1H, m), 7.6 (1H, m), 7.3 (1H, m), 7.0 (2H, 2 × s), 5.6 (1H, m), 3.9 (1H, m), 3.7 (1H, m), 2.9 (6H, 2 × s) | 419.0 (M + H)⁺ |
| #14 | 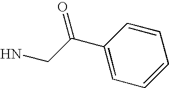 | (CDCl₃) 9.6 (1H, s), 7.0-8.0 (8H, m), 5.0 (2H, m) | 317.1 |
| #15 | 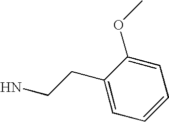 | (CDCl₃) 9.6 (1H, br), 7.2 (2H, m), 7.0 (1H, s), 6.9 (2H, m), 6.6 (1H, s), 6.3 (1H, br), 3.9 (3H, s), 3.7 (2H, m), 2.9 (2H, t) | 333.1 |
| #16 | 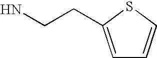 | (CDCl₃) 9.8 (1H, br), 6.9-7.2 (4H, m), 6.7 (1H, s), 6.2 (1H, br), 3.8 (2H, m), 3.2 (2H, t) | 309.1 |
| #17 | 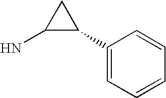 | (CDCl₃) 9.6 (1H, br), 7.0-7.3 (6H, m), 6.8 (1H, s), 6.3 (1H, br), 3.1 (1H, m), 2.2 (1H, m), 1.3 (2H, m) | 315.1 |
| #18 | 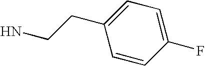 | (CDCl₃) 9.5 (1H, br), 7.0-7.2 (5H, m), 6.6 (1H, s), 5.9 (1H, br), 37 (2H, m), 2.9 (2H, t) | 321.1 |
| #19 | 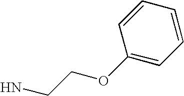 | (CDCl₃) 9.6 (1H, br), 6.9-7.3 (6H, m), 6.8 (1H, s), 6.5 (1H, m), 4.2 (2H, t), 3.8 (2H, m) | 319.1 |
| #20 | 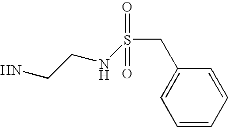 | (CDCl₃) 9.6 (1H, s), 6.8-7.4 (9H, m), 4.3 (2H, s), 3.4 (2H, m), 3.1 (2H, m) | 396.1 |
| #21² | 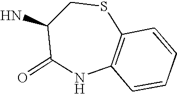 | (CDCl₃) 9.7 (1H, s), 8.0 (1H, s), 7.7 (1H, d), 7.1-7.4 (4H, m), 7.0 (1H, s), 6.9 (1H, s), 4.9 (1H, m), 4.0 (1H, m), 3.0 (1H, m) | 378.1 (M + H)⁺ |

¹Amine: Method #15
²Amine: J Med Chem, 1985, 28, 1517

### Example #22

### 2-Chloro-5-[N-(benzoylmethyl)carbamoyl]-4H-thieno[3,2-b]pyrrole

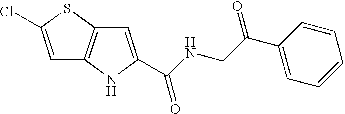

5-Carboxy-2-chloro-4H-thieno[3,2-b]pyrrole (Method #8; 50 mg, 0.25 mmol) was dissolved in DCM (7 ml) containing HOBT (34 mg, 0.25 mmol), DIPEA (49 ml, 0.28 mmol) and 2-amino-1-phenylethanone (43 mg, 0.25 mmol). The mixture was stirred for one minute and EDAC (63 mg, 0.33 mmol) was added. The mixture was stirred at room temperature for approximately 18 hours. Water was added to the solution and a solid precipitated out. This solid was filtered off and was washed with water and DCM before being dried under reduced pressure to afford the title compound as a white solid (45 mg, 61%). NMR: 11.9 (1H, s), 8.6 (1H, t), 7.1-8.1 (7H, m), 4.8 (2H, d); m/z 317.3.

### Examples #23-#2

The following compounds were made by the process of Example #22 using 5-carboxy-2-chloro-4H-thieno[3,2-b]pyrrole (Method #8) and the appropriate amine:

### Example #23

### 2-Chloro-5-1N-(2-phenyl-1-cyclopropyl)carbamoyl]-4H-thieno[3,2-b]pyrrole

### Example #24

### 2-Chloro-5-[N-(N-phenylcarbamoylmethyl)carbamoyl]-4H-thieno[3,2-b]pyrrole

### Example #25

### 2-Chloro-5-(N-{2-[(2-pyridyl)amino]ethyl}carbamoyl)-4H-thieno[3,2-b]pyrrole

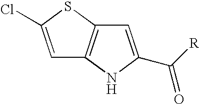

| Ex | R | NMR | M/z |
|---|---|---|---|
| #23 | 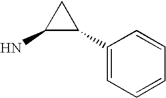 | 11.8 (1H, s), 8.5 (1H, d), 7.0-7.3 (7H, m), 3.0 (1H, m), 2.1 (1H, m), 1.3 (2H, m) | 315.3 |
| #24 | 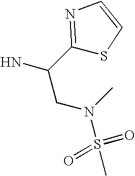 | 11.8 (1H, s), 10.0(1H, br), 8.6 (1H, br), 7.0-7.6 (7H, m), 4.0 (2H, d) | 332.2 |
| #25¹ | 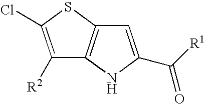 | 11.8 (1H, s), 8.4 (1H, s), 6.4-8.0 (6H, m), 6.6 (1H, br), 3.4 (4H, m) | 319.3 |

¹Amine: Eur J Med Chem, 1987, 22, 91.

### Example #26

### 2-Chloro-5-{N-[2-(2-methoxyphenyl)ethyl]carbamoyl}-4H-thieno[3,2-b]pyrrole

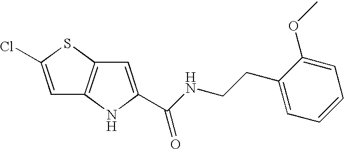

5-Carboxy-2-chloro-4H-thieno[3,2-b]pyrrole (Method #8; 50 mg, 0.25 mmol) was dissolved in DCM (7 ml) containing HOBT (34 mg, 0.25 mmol), DIPEA (49 ml, 0.28 mmol) and 2-(2-methoxyphenyl)ethylamine (43 mg, 0.25 mmol). The mixture was stiffed for one minute and EDAC (63 mg, 0.33 mmol) was added. The mixture was stirred at room temperature for approximately 18 hours. The reaction mixture was washed with water, the organic phase was dried, filtered and concentrated to afford the title compound as an off-white solid (84 mg, 100%). NMR (CDCl₃): 9.7 (1H, br), 6.9-7.2 (5H, m), 6.5 (1H, s), 6.3 (1H, br), 3.9 (3H, s), 3.7 (2H, m), 3.0 (2H, t); m/z 333.4.

### Examples #27-#31

The following compounds were made by the process of Example #26 using 5-carboxy-2-chloro-4H-thieno[3,2-b]pyrrole (Method #8) and the appropriate amine.

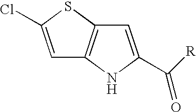

### Example #27

### 2-Chloro-5-[N-(2-phenoxyethyl)carbamoyl]-4H-thieno[3,2-b]pyrrole

### Example #28

### 2-Chloro-5-{N-[2-(2-thienyl)ethyl]carbamoyl}-4H-thieno[3,2-b]pyrrole

### Example #29

### 2-Chloro-5-{N-[2-(4-fluorophenyl)ethyl]carbamoyl}-4H-thieno[3,2-b]pyrrole

### Example #30

### 2-Chloro-5-{N-[2-(N-methylmethanesulphonamido)-1-(thiazol-2-yl)ethyl]carbamoyl}-4H-thieno[3,2-b]pyrrole

### Example #31

### 2-Chloro-5-{N-[2-(thiomorpholino)ethyl]carbamoyl}-4H-thieno[3,2-b]pyrrole

| | | | |
|---|---|---|---|
| Ex | R | NMR | M/z |

| | | | |
|---|---|---|---|
| #27 | 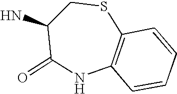 | (CDCl₃) 9.5 (1H, s), 6.9-7.3 (6H, m), 62 (1H, s), 6.4 (1H, br) 4.2 (2H, m), 3.9 (2H, m) | |
| #28 | 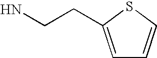 | (CDCl₃) 9.4 (1H, br), 6.6-7.2 (5H, m), 6.0 (1H, br), 3.7 (2H, m), 3.1 (2H, m) | |
| #29 | 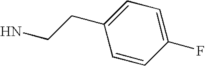 | (CDCl₃) 9.4 (1H, s), 7.0-7.2 (4H, m), 6.9 (1H, s), 6.6 (1H, s), 5.9 (1H, br), 3.7 (2H, m), 2.9 (2H, t) | 321.3 |
| #30¹ | 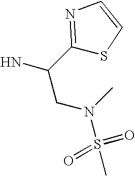 | 11.9 (1H, br), 9.0 (1H, d), 7.8 (1H, m), 7.7 (1H), 7.2 (1H, s), 7.0 (1H, s), 5.6 (1H, m), 3.9 (1H, m), 3.6 (1H, m), 2.9 (3H, s), 2.8 (3H, s) | 417.2 |
| #31 | 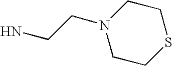 | 11.8 (1H, s), 8.1 (1H, t), 6.8-7.4 (2H, m),l 2.5-4.0 (12H, m) | 328.3 |

⁶Amine: Method #15

### Examples #32-#69

The following compounds were made by the process of Example #1 using 5-carboxy-2,3-dichloro-4H-thieno[3,2-b]pyrrole (Method #9) or Example #22 using 5-carboxy-2-chloro-4H-thieno[3,2-b]pyrrole (Method #8) and the appropriate amine:

### Example #32

### 2,3-Dichloro-5-[N-(2,3-dimethyl-5-oxo-1-phenyl-2,5-dihydro-1H-pyrazol-4-yl)carbamoyl]-4H-thieno[3,2-b]pyrrole

### Example #33

### 2,3-Dichloro-5-[N-(4-sulphamoylphenylmethyl)carbamoyl]-4H-thieno[3,2-b]pyrrole

### Example #34

### 2,3-Dichloro-5-[N-(2-hydroxy-1-phenethyl)carbamoyl]-4H-thieno[3,2-b]pyrrole

### Example #35

### 2,3-Dichloro-5-{N-(2-[(3-trifluoromethylpyrid-2-yl)amino]ethyl)carbamoyl}-4H-thieno[3,2-b]pyrrole

### Example #36

### 2,3-Dichloro-5-{N-[3-(5-tetrazolyl)propyl]carbamoyl}-4H-thieno[3,2-b]pyrrole

### Example #37

### 2,3-Dichloro-5-[N-(5-oxo-3-phenyl-4,5-dihydroisoxazol-4-yl)carbamoyl]-4H-thieno[3,2-b]pyrrole

### Example #38

### 2,3-Dichloro-5-[N-(5-hydroxy-2-oxo-2,3,4,5-tetrahydro-1H-benz[b]azepin-4-yl)carbamoyl]-4H-thieno[3,2-b]pyrrole

### Example #39

### 2-Chloro-5-{N-[3-(benzyloxycarbonylamino)propyl]carbamoyl}-4H-thieno[3,2-b]pyrrole

### Example #40

### 2,3-Dichloro-5-{N-[(4-dimethylaminophenyl)methyl]carbamoyl}-4H-thieno[3,2-b]pyrrole

### Example #41

### 5-[N-(1-Benzyl-2-hydroxyethyl)carbamoyl]-2,3-dichloro-4H-thieno[3,2-b]pyrrole

### Example #42

### 2,3-Dichloro-5-{N-[2-(phenylamino)ethyl]carbamoyl}-4H-thieno[3,2-b]pyrrole

### Example #43

### 2,3-Dichloro-5-[N-(β-(R)-hydroxy-α-methylphenethyl)carbamoyl]-4H-thieno[3,2-b]pyrrole

### Example #44

### 2,3-Dichloro-5-[N-(β-hydroxyphenethyl)carbamoyl)-4H-thieno[3,2-b]pyrrole

### Example #45

### 2,3-Dichloro-5-{N-[2-(4-hydroxyphenyl)ethyl]carbamoyl}4H-thieno[3,2-b]pyrrole

### Example #46

### 2,3-Dichloro-5-{N-[(benzimidazol-2-yl)methyl]carbamoyl}-4H-thieno[3,2-b]pyrrole

### Example #47

### 2,3-Dichloro-5-{N-[2-(4-chlorophenyl)-2-hydroxy-1-(methoxycarbonyl)ethyl]carbamoyl}-4H-thieno[3,2-b]pyrrole

### Example #48

### 2,3-Dichloro-5-{N-(imidazo[1,2-a]pyrid-2-yl)carbamoyl}-4H-thieno[3,2-b]pyrrole

### Example #49

### 5-{N-[(Benzthiazol-2-yl)methyl]carbamoyl}-2,3-dichloro-4H-thieno[3,2-b]pyrrole

### Example #50

### 2,3-Dichloro-5-{N-[(6-trifluoromethylpyrid-3-yl)methyl]carbamoyl}-4H-thieno[3,2-b]pyrrole

### Example #51

### 2,3-Dichloro-5-{N-(2-[(2-pyridazinyl)methyl]carbamoyl}-4H-thieno[3,2-b]pyrrole

### Example #52

### 2,3-Dichloro-5-{N-[N-(2-hydroxy-3-phenoxypropyl)carbamoylmethyl carbamoyl}-4H-thieno[3,2-b]pyrrole

### Example #53

### 2,3-Dichloro-5-{N-[N-(3-methylisothiazol-5-yl)carbamoylmethyl]carbamoyl}-4H-thieno[3,2-b]pyrrole

### Example #54

### 2,3-Dichloro-5-{N-[2-(pyridazin-3-yloxy)ethyl]carbamoyl}-4H-thieno[3,2-b]pyrrole

### Example #55

### 2-Chloro-5-(N-{2-[(3-trifluoromethylpyrid-2-yl)amino]ethyl}carbamoyl)-4H-thieno[3,2-b]pyrrole

### Example #56

### 2,3-Dichloro-5-{N-[2-(4-sulphamoylphenyl)ethyl]carbamoyl}-4H-thieno[3,2-b]pyrrole

### Example #57

### 2,3-Dichloro-5-{N-[2-(2-pyridyl)ethyl]carbamoyl)-4H-thieno[3,2-b]pyrrole

### Example #58

### 2,3-Dichloro-5-{N-(2-[1-hydroxymethyl-2-(4-imidazolyl)ethyl]carbamoyl}-4H-thieno[3,2-b]pyrrole

### Example #59

### 2,3-Dichloro-5-{N-(2-[(3-quinolyl)methyl]carbamoyl}-4H-thieno[3,2-b]pyrrole

### Example #60

### 5-{N-[3-(4-Acetamidophenoxy)-2-hydroxypropyl]carbamoyl}-2,3-dichloro-4H-thieno[3,2-b]pyrrole

### Example #61

### 2,3-Dichloro-5-{N-[3-(N-methylsulphonylcarbamoyl)propyl]carbamoyl}-4H-thieno[3,2-b]pyrrole

### Example #62

### 2,3-Dichloro-5-[N-(2-{[2-(guanidino)thiazol-4-yl]methylthio}ethyl)-carbamoyl]-4H-thieno[3,2-b]pyrrole

### Example #63

### 2,3-Dichloro-5-{N-[2-(2,4-dioxoimidazolidin-1-yl)ethyl]carbamoyl}-4H-thieno[3,2-b]pyrrole

### Example #64

### 5-{N-[2-Benzylthio-1-(hydroxymethyl)ethyl]carbamoyl}-2,3-dichloro-4H-thieno[3,2-b]pyrrole

### Example #65

### 2,3-Dichloro-5-{N-[2-(dimethyaminosulphonylamino)ethyl]carbamoyl}-4H-thieno[3,2-b]pyrrole

### Example #66

### 2,3-Dichloro-5-{N-[(6-methoxypyrid-3-yl)methyl]carbamoyl}-4H-thieno[3,2-b]pyrrole

### Example #67

### (S)-2,3-Dichloro-5-{N-[(2-oxo-3-phenyl-2,3,4,5-tetrahydrooxazol-5-yl)-methyl]carbamoyl}-4H-thieno[3,2-b]pyrrole

### Example #68

### 2,3-Dichloro-5-(N-{2-[3-(carbamoylmethyl)phenoxy]ethyl}carbamoyl)-4H-thieno[3,2-b]pyrrole

### Example #69

### 5-(N-{[6-(Benzo[1,3]dioxol-5-yl)-4-methylmorpholin-2-yl]methyl}-carbamoyl)-2,3-dichloro-4H-thieno[3,2-b]pyrrole

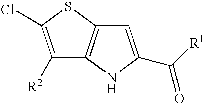

| Ex | R¹ | R₂ | M/z (M + H)⁺ | Amine |
|---|---|---|---|---|
| 32 | 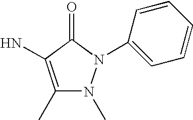 | Cl | 421 | commercial available |
| 33 | 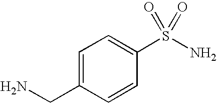 | Cl | 404 | commercial available |
| 34 | 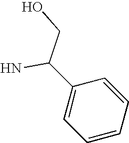 | Cl | 355 | commercial available |
| 35 | 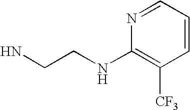 | Cl | 423 | commercial available |
| 36 | 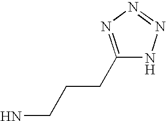 | Cl | 345 | Meth #19 |
| 37 | 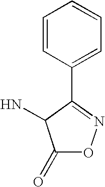 | Cl | 394 | Gazz. Chim. Ital., 1981, 111, 167 |
| 38 | 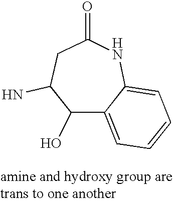 | Cl | 410 | Method #26 |
| 39 | 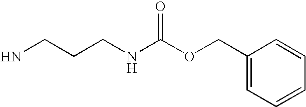 | H | 393 | commercial available |
| 40 | 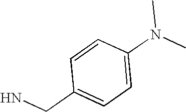 | Cl | 368 | commercial available |
| 41 | 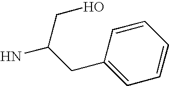 | Cl | 369 | commercial available |
| 42 | 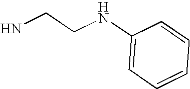 | Cl | 354 | commercial available |
| 43 | 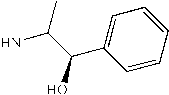 | Cl | 369 | commercial available |
| 44 | 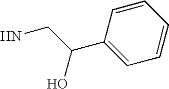 | Cl | 355 | commercial available |
| 45 | 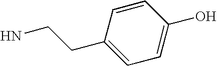 | Cl | 355 | commercial available |
| 46 | 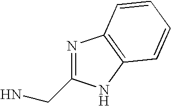 | Cl | 365 | commercial available |
| 47 | 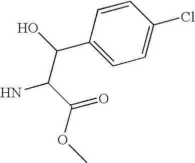 | Cl | 448 | commercial available |
| 48 | 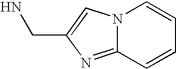 | Cl | 365 | commercial available |
| 49 | 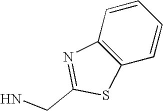 | Cl | 382 | commercial available |
| 50 | 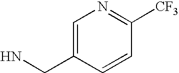 | Cl | 394 | commercial available |
| 51 | 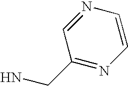 | Cl | 327 | commercial available |
| 52 | 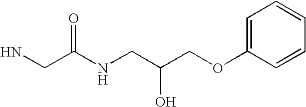 | Cl | 442 | Method #20 |
| 53 | 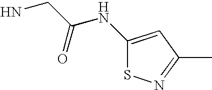 | Cl | 389 | Method #21 |
| 54 | 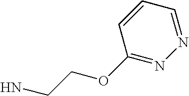 | Cl | 357 | Method #22 |
| 55 | 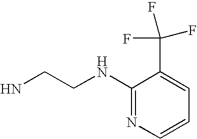 | Cl | 388 | commercial available |
| 56 | 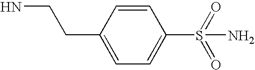 | Cl | 418 | commercial available |
| 57 | 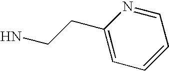 | Cl | 340 | commercial available |
| 58 | 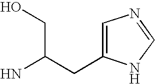 | Cl | 359 | commercial available |
| 59 | 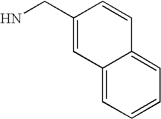 | Cl | 376 | commercial available |
| 60 | 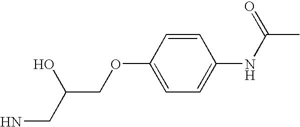 | Cl | 442 | commercial available |
| 61 | 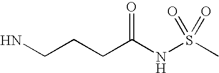 | Cl | 398 | Method #20 |
| 62 | 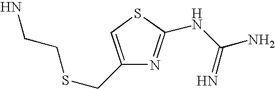 | Cl | 449 | Eur. J. Med. Chem., 1993, 28, 601 |
| 63 | 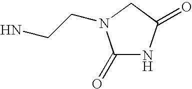 | Cl | 361 | Method #23 |
| 64 | 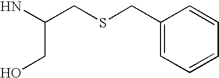 | Cl | 415 | commercial available |
| 65 | 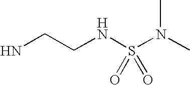 | Cl | 385 | commercial available |
| 66 | 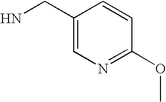 | Cl | 356 | WO 9518097 |
| 67 | 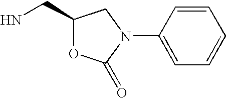 | Cl | 410 | J. Med. Chem., 1989, 32, 1673 |
| 68 | 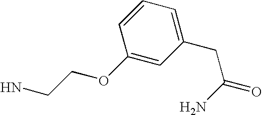 | Cl | 412 | Method #25 |
| 69 | 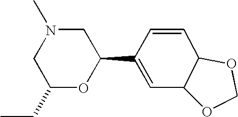 | Cl | 468 | Method #27 |

### Example #70

### 5-(N-Benzylcarbamoyl)-2,3-dichloro-4H-thieno[3,2-b]pyrrole

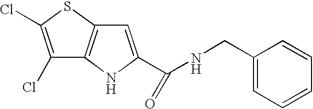

5-Carboxy-2,3-dichloro-4H-thieno[3,2-b]pyrrole (Method #9, 118 mg, 0.5 mmol) was dissolved in dichloromethane (10 ml) containing benzylamine (55 mg, 0.5 mmol), 1-HOBT (68 mg, 0.5 mmol) and DIPEA (258 μl, 2 mmol). The mixture was stirred for one minute before the addition of EDAC (125 mg, 0.65 mmol). The mixture was stirred at ambient temperature for approximately 16 hours before being washed with water. The organic fraction was concentrated and was purified using Bond-Elut silica column chromatography (eluent: dichloromethane-dichloromethane/methanol 5% gradient) to afford the title compound as a white solid (121 mg, 75%).

NMR: 12.4 (1H, br), 8.8 (1H, t), 7.3 (5H, m), 7.1 (1H, s), 4.5 (2H, d); m/z 323.27

The following compounds were made by the process of Example #70 using 5-carboxy-2,3-dichloro-4H-thieno[3,2-b]pyrrole (Method #9) and the appropriate amine:

### Example #71

### 2,3-Dichloro-5-(N-phenethylcarbamoyl)-4H-thieno[3,2-b]pyrrole

### Example #72

### 2,3-Dichloro-5-[N-(3-phenylpropyl)carbamoyl]-4H-thieno[3,2-b]pyrrole

### Example #73

### 2,3-Dichloro-5-{N-[2-(2-hydroxyphenyl)ethyl]carbamoyl}-4H-thieno[3,2-b]pyrrole

### Example #74

### 2,3-Dichloro-5-[N-(α,α-dimethylphenethyl)carbamoyl]-4H-thieno[3,2-b]pyrrole

### Example #75

### 2,3-Dichloro-5-[N-(1-phenylcyclobutyl)methyl)carbamoyl]-4H-thieno[3,2-b]pyrrole

### Example #76

### 2,3-Dichloro-5-[N-(β-methylphenethyl)carbamoyl]-4H-thieno[3,2-b]pyrrole

### Example #77

### 2,3-Dichloro-5-[N-(1,2,3,4-tetrahydronaphth-2-yl)carbamoyl]-4H-thieno[3,2-b]pyrrole

### Example #78

### 5-[N-(N-Benzylcarbamoylmethyl)carbamoyl]-2,3-dichloro-4H-thieno[3,2-b]pyrrole

### Example #79

### 5-[N-(N-Benzyl-N-methylcarbamoylmethyl)carbamoyl]-2,3-dichloro-4H-thieno[3,2-b]pyrrole

### Example #80

### 2,3-dichloro-5-[N-(N-methyl-N-phenylcarbamoylmethyl)carbamoyl]-4H-thieno[3,2-b]pyrrole

### Example #81

### 2,3-dichloro-5-{N-[N-(2-cyanoethyl)-N-phenylcarbanoylmethyl]carbamoyl}-4H-thieno[3,2-b]pyrrole

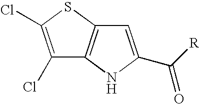

| Ex | R | NMR | M/z |
|---|---|---|---|
| #71 | 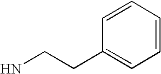 | 12.33(1H, br), 8.4(1H, t), 7.3(5H, m), 7.1(1H, s), 3.5(2H, dd), 2.8(2H, t). | 337.38 |
| #72 | 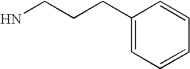 | 12.35(1H, br), 8.3(1H, t), 7.2(5H, m), 7.1(1H, s), 3.3(2H, q), 2.6(2H, t), 1.8(2H, m). | |
| #73 | 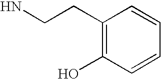 | 12.3(1H, br), 8.3(1H, t), 7.0(3H, m), 6.8(1H, d), 6.7(1H, t), 3.4(3H, q), 2.8(2H, t) | 353.39 |
| #74¹ | 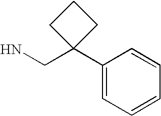 | 12.3(1H, br), 8.1(1H, t), 7.3(2H, t), 7.15(3H, m), 7.05(1H, s), 3.6(2H, d), 2.4(2H, m), 2.2(2H, m), 2.0(2H, m), 1.8(1H, m). | 377.44 |
| #75² | 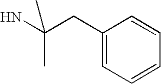 | 12.25(1H, br), 7.4(1H, s), 7.1(6H, m), 3.1(2H, s), 1.3(6H, s) | 365.41 |
| #76³ | 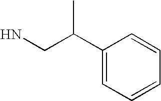 | 12.3(1H, br), 8.3(1H, t), 7.2(5H, m), 7.0(1H, s), 3.4(2H, m), 3.0(1H, q), 1.2(3H, d). | no mass ion |
| #77 | 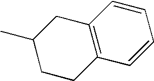 | 12.35(1H, br), 8.2(1H, d), 7.1(1H, s), 7.05(4H, s), 4.2(1H, br), 3.1(1H, dd), 2.8(3H, m), 2.0(1H, m), 1.8(1H, m). | 363.35 |
| #78⁴ | 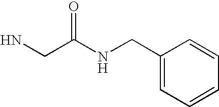 | 12.4(1H, br), 8.6(1H, t), 8.4(1H, t), 7.3(5H, m), 4.3(2H, d), 3.9(2H, d). | 380.41 |
| #79⁵ | 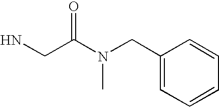 | 12.45(1h, br), 8.4(1H, m), 7.3(5H, m), 7.2(1H, m), 4.6(0.6, s), 4.5(1.4H, s), 4.2(2H, m), 3.0(2H, s), 2.8(1H, s) | 394.44 |
| #80⁶ | 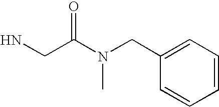 | 12.4(1H, br), 8.4(1H, t), 7.4(5H, m), 7.1(1H, s), 3.8(2H, br), 3.2(3H, s). | 380.41 |
| #81⁷ | 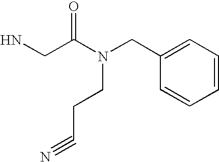 | 12.4(1H, br), 8.4(1H, t), 7.5(5H, m), 7.1(1H, s), 3.9(2H, t), 3.7(2H, br), 2.7(2H, t). | 419.41 |

¹Amine: J. Org. Chem.; 1976, 41(14), 2502-2503
²Amine: J. Med. Chem.; 1993, 36(22), 3300-3307
³Amine: J. am. Chem. Soc.; 1960, 82, 2577
⁴Amine: Method #6
⁵Amine: Method #31
⁶Amine: Method #30
⁷Amine: Method #14

### Example #82

### 2,3-dichloro-5-{N-[N-(4-methoxyphenyl)carbamoylmethyl]carbamoyl}-4H-thieno[3,2-b]pyrrole

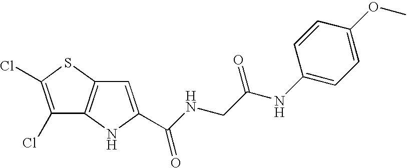

A solution of 5-(N-carboxymethylcarbamoyl)-2,3-dichloro-4H-thieno[3,2-b]pyrrole (Method #12, 150 mg, 0.51 mmol) and 4-methoxyaniline (69 mg, 0.56 mmol) in tetrahydrofuran (THF) (6 ml) was stirred at ambient temperature for 30 minutes. 4-(4,6-dimethoxy-1,3,5-triazin-2-yl)-4-methylmorpholinium chloride (DMTMM) (142 mg, 0.51 mmol) was added and the reaction mixture stirred at ambient temperature overnight, poured into water (15 ml) and extracted with ethyl acetate (3×15 ml). The organic extracts were combined and washed with 1N citric acid solution (15 ml), sodium bicarbonate solution (15 ml), dried over magnesium sulphate, filtered and concentrated to give the title product as a white solid NMR: 12.4 (1H, s), 9.9 (1H, s), 8.6 (1H, t), 7.5 (2H, d), 7.2 (1H, s), 6.85 (2H, d), 4.0 (2H, d), 3.7 (3H, s); m/z 396.38

The following compounds were made by the process of Example #82 using 2,3-dichloro-5-[N-carboxymethylcarbamoyl]-4H-thieno[3,2-b]pyrrole (Method #12) and the appropriate commercially available amine:

### Example #83

### 2,3-dichloro-5-{N-[N-(4-fluorophenyl)carbamoylmethyl]carbamoyl}-4H-thieno[3,2-b]pyrrole

### Example #84

### 2,3-dichloro-5-{N-[N-(4-nitrophenyl)carbamoylmethyl]carbamoyl}-4H-thieno[3,2-b]pyrrole

### Example #85

### 2,3-dichloro-5-{N-[N-(2,6-dimethylphenyl)carbamoylmethyl]carbamoyl}-4H-thieno[3,2-b]pyrrole

### Example #86

### 2,3-dichloro-5-{N-[N-methyl-N-(4-methylphenyl)carbamoylmethyl]-carbamoyl}-4H-thieno[3,2-b]pyrrole

### Example #87

### 2,3-dichloro-5-{N-[N-methyl-N-(3-methylphenyl)carbamoylmethyl]-carbamoyl}-4H-thieno[3,2-b]pyrrole

### Example #88

### 2,3-dichloro-5-{N-[N-(3-chlorophenyl) N-methylcarbamoylmethyl]-carbamoyl}-4H-thieno[3,2-b]pyrrole

### Example #89

### 2,3-dichloro-5-{N-[N-(2-hydroxyethyl)-N-phenylcarbamoylmethyl]-carbamoyl}-4H-thieno[3,2-b]pyrrole

### Example #90

### 2,3-dichloro-5-{N-[N-(1,1-dimethyl-2-hydroxyethyl)carbamoylmethyl]-carbamoyl}-4H-thieno[3,2-b]pyrrole

### Example #91

### 2,3-dichloro-5-{N-[N-(2-hydroxyethyl)-N-methylcarbamoylmethyl]-carbamoyl}-4H-thieno[3,2-b]pyrrole

### Example #92

### 2,3-dichloro-5-{N-[N-(2-hydroxyethyl)carbamoylmethyl]carbamoyl}-4H-thieno[3,2-b]pyrrole

### Example #93

### 2,3-dichloro-5-{N-[N-(3-hydroxypropyl)carbamoylmethyl]carbamoyl}-4H-thieno[3,2-b]pyrrole

### Example #94

### 2,3-dichloro-5-{N-[N-(4-hydroxybutyl)carbamoylmethyl]carbamoyl}-4H-thieno[3,2-b]pyrrole

### Example #95

### 2,3-dichloro-5-(N-{N-[bis(hydroxymethyl)methyl]carbamoylmethyl}-carbamoyl)-4H-thieno[3,2-b]pyrrole

### Example #96

### 2,3-dichloro-5-{N-[N-(2,3-dihydroxypropyl)carbamoylmethyl]carbamoyl}-4H-thieno[3,2-b]pyrrole

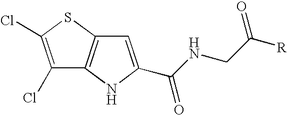

| Ex | R | NMR | m/z |
|---|---|---|---|
| #83 | 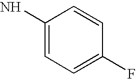 | 12.42(1H, br), 10.08(1H, br), 8.6(1H, t), 7.6(2H, m), 7.1(3H, m), 4.0(2H, d) | 384.20 |
| #84 | 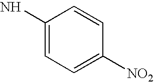 | 12.5(1H, br), 10.74(1H, br), 8.8(1H, t), 8.3(2H, d), 7.9(2H, d), 7.2(1H, s), 4.2(2H, d). | 411.36 |
| #85 | 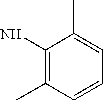 | 12.45(1H, br), 9.3(1H, br), 8.7(1H, t), (1H, s), 7.1(3H, s), 4.1(2H, d), 2.1(6H, s). | 394.39 |
| #86 | 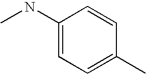 | 12.4(1H, br), 8.4(1H, t), 7.3(4H, s), 7.1(1H, s), 3.7(2H, br), 3.2(3H, s), 2.3(3H, s) | 394.17 |
| #87 | 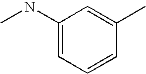 | 12.4(1H, br), 8.4(1H, t), 7.4(1H, t), 7.2(3H, m), 7.1(1H, s), 3.8(2H, br), 3.3(3H, s), 2.3(3H, s) | 394.28 |
| #88 | 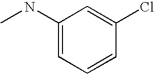 | 12.4(1H, br), 8.4(1H, t), 7.5(4H, m), 7.1(1H, s), (3.9, br), 3.2(3H, s). | no mass ion |
| #89 | 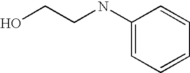 | 12.4(1H, br), 8.4(1H, t), 7.4(5H, m), 7.1(1H, s), 4.7(1H, br), 3.7(4H, m), 3.5(2H, d). | 410.54 |
| #90 | 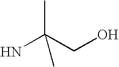 | 12.4(1H, br), 8.4(1H, t), 7.3(1H, br), 7.1(1H, s), 4.8(1H, t), 3.8(2H, d), 3.4(1.3H, d), 3.1(0.7H, d), 1.2(6H, s) | 362.33 |
| #91 | 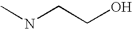 | 12.44(1H, br), 8.3(1H, br), 7.1(1H, s), 4.9(0.5H, t), 4.6(0.5H, t), 4.1(2H, dd), 3.4(4H, m), 3.0(1.5H, s), 2.8(0.5H, s). | 348.30 |
| #92 | 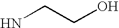 | 12.42(1H, br), 8.5(1H, t), 7.8(1H, t), 7.1(1H, s), 4.6(1H, br), 3.8(2H, d), 3.4(2H, br), 3.1(2H, q). | 334.34 |
| #93 | 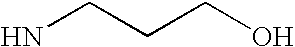 | 12.41(1H, br), 8.5(1H, t), 7.8(1H, t), 7.1(1H, s), 4.4(1H, t, br), 3.8(2H, d), 3.4(2H, q, br), 3.1(2H, q), 1.5(2H, m). | 348.38 |
| #94 | 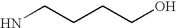 | 12.4(1H, br), 8.5(1H, t), 7.9(1H, t), 7.1(1H, s), 4.3(1H, t), 3.8(2H, d), 3.4(2H, d), 3.1(2H, q, br), 1.4(4H, m). | 362.36 |
| #95 | 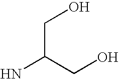 | 12.42(1H, br), 8.5(1H, t), 7.6(1H, d), 7.1(1H, s), 4.6(2H, br), 3.9(2H, d), 3.7(1H, m), 3.4(4H, br). | 364.36 |
| #96 | 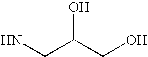 | 12.4(1H, br), 8.5(1H, br), 7.8(1H, t), 7.1(1H, s), 4.7(1H, br) 4.5(1H, br), 3.8(2H, d), 3.5(1H, m), 3.2(3H, m), 3.0(1H, m). | 364.35 |

### Example #97

### 2,3-dichloro-5-{N-[N-(4-hydroxymethylphenyl)carbamoymethyl]carbamoyl}-4H-thieno[3,2-b]pyrrole

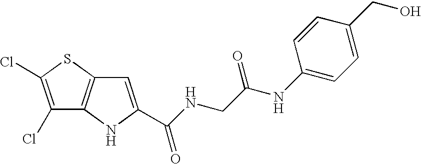

A solution of 5-(N-carboxymethylcarbamoyl)-2,3-dichloro-4H-thieno[3,2-b]pyrrole (Method #12) (150 mg, 0.51 mmol), and 4-aminobenzyl alcohol (70.5 mg, 0.56 mmol) in THF (6 ml) was stirred at ambient temperature for 30 minutes. 4-(4,6-dimethoxy-1,3,5-triazin-2-yl)-4-methylmorpholinium chloride (DMTMM) (142 mg, 0.51 mmol) was added and the reaction mixture stirred at ambient temperature overnight, then poured into water (15 ml). The resultant precipitate was isolated by filtration, washed with water, ether and dried in vacuo to give the title product as a white solid (149 mg, 73%)

NMR: 12.42 (1H, br), 9.9 (1H, s), 8.6 (1H, t), 7.5 (2H, d), 7.2 (2H, d), 7.1 (1H, s), 5.0 (1H, br), 4.4 (2H, s), 4.0 (2H, d); m/z 396.21

The following compounds were made by the process of Example #97 using 5-(N-carboxymethylcarbamoyl)-2,3-dichloro-4H-thieno[3,2-b]pyrrole (Method #12) and the appropriate commercially available amine:

### Example #98

### 2,3-dichloro-5-{N-[N-(5-isoquinolyl)carbamoylmethyl}carbamoyl}-4H-thieno[3,2-b]pyrrole

### Example #99

### 2,3-dichloro-5-{N-[N-(3-hydroxymethy)lphenyl]carbamoylmethyl]carbamoyl}-4H-thieno[3,2-b]pyrrole

### Example #100

### 2,3-dichloro-5-(N-{N-[4-(2-hydroxyethyl)phenyl]carbamoylmethyl}carbamoyl)-4H-thieno[3,2-b]pyrrole

### Example #101

### 2,3-dichloro-5-{N-[N-(2,4-difluorophenyl)-N-methyl-carbamoylmethyl]-carbamoyl}-4H-thieno[3,2-b]pyrrole

### Example #102

### 2,3-dichloro-5-{N-[(1,2,3,4-tetrahydro-1-quinolyl)carbonylmethyl]-carbamoyl}-4H-thieno[3,2-b]pyrrole

### Example #103

### 2,3-dichloro-5-{N-[N-(2-cyanoethyl)-N-methylcarbamoylmethyl]-carbamoyl}-4H-thieno[3,2-b]pyrrole

### Example #104

### 2,3-dichloro-5-{N-[N-(4-hydroxypiperidino)carbamoylmethyl]carbamoyl}-4H-thieno[3,2-b]pyrrole

### Example #105

### 2,3-dichloro-5-[N-(N-cyclopentylcarbamoylmethyl)carbamoyl]-4H-thieno[3,2-b]pyrrole

### Example #106

### 2,3-dichloro-5-[N-(N-isopropylcarbamoylmethyl)carbamoyl]-4H-thieno[3,2-b]pyrrole

### Example #107

### 2,3-dichloro-5-[N-(N-isopropyl-N-methylcarbamoylmethyl)carbamoyl]-4H-thieno[3,2-b]pyrrole

### Example #108

### 2,3-dichloro-5-[N-(thiomorpholinocarbonylmethyl)carbamoyl]-4H-thieno[3,2-b]pyrrole

### Example #109

### 2,3-dichloro-5-[N-(morpholinocarbonylmethyl)carbamoyl]-4H-thieno[3,2-b]pyrrole

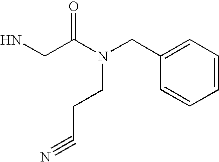

| Ex | R | NMR | m/z |
|---|---|---|---|
| #98 | 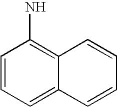 | 12.5(1H, s), 8.7(1H, t), 8.5(1H, d), 8.0(3H, m), 7.7(1H, t), 7.1(1H, s), 4.2(2H, d). | |
| #99 | 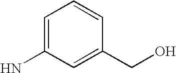 | 12.42(1H, br), 9.9(1H, s), 8.691H, t), 7.55(1H, s), 7.4591H, d), 7.2(1H, t), 7.1(1H, s), 7.0(1H, d), 5.1(1H, br), 4.4(2H, s), 4.0(2H, d) | 396.19 |
| #100 | 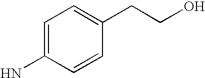 | 12.41(1H, br), 9.9(1H, s), 8.6(1H, t), 7.5(1H, m), 7.1(3H, m), 4.6(1H, br), 4.0(2H, d), 3.6(2H, br), 2.6(2H, t) | 410.16 |
| #101 | 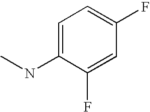 | 12.4(1H, br), 8.5(1H, t), 7.7(1H, q), 7.5(1H, dt), 7.2(1H, t, br), 7.1(1H, s), 3.8(1H, m), 3.6(1H, m), 3.1(3H, s). | No mass ion |
| #102 | 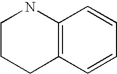 | 12.41(1H, br), 8.5(1H, t), 7.691H, d), 7.1(5H, m), 4.2(2H, d), 3.7(2H, t), 2.7(2H, t), 1.9(2H, m). | 406.18 |
| #103 | 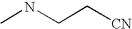 | 12.42(1H, br), 8.4(1H, m), 7.1(1H, s), 4.1(2H, m), 3.7(0.5H, t), 3.6(1.5H, t), 3.1(2H, s), 2.9(1.5H. m), 2.7(1.5H, m), 2.7(1.5H, t). | 357.27 |
| #104 | 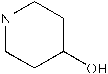 | 12.42(1H, br), 8.4(1H, t), 7.1(1H, s), 4.7(1H, br), 4.1(2H, d), 3.9(1H, m), 3.7(2H, m), 3.2(1H, m), 3.0(3H, m), 1.7(2H, m), 1.3(2H, m). | 376.26 |
| #105 | 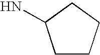 | 12.4(1H, br), 8.45(1H, t), 7.8(1H, d), 7.1(1H, s), 4.0(1H, m), 3.8(2H, d), 1.8 (2H, m), 1.6(2H, m), 1.5(2H, m), 1.4(2H, m). | 358.35 |
| #106 | 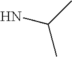 | 12.4(1H, br), 8.5(1H, t), 7.7(1H, d), 7.1(1H, s), 3.9(1H, m), 3.8(2H, d), 1.1(6H, d). | 358.35 |
| #107 | 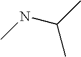 | 12.42(1H, br), 8.3(1Hm ), 7.1(1H, s), 4.6(0.66H, m), 4.1(2.33H, m), 2.8(2H, s), 2.7(1H, s), 1.15(2.4H, d), 1.05(3.6H, d). | 346.34 |
| #108 | 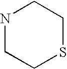 | 12.42(1H, br), 8.4(1H, t), 7.1(1H, s), 4.2(1H, d), 3.7(4H, m), 2.7(2H, br), 2.5(2H, br). | no mass ion |
| #109 | 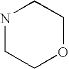 | 12.42(1H, br), 8.4(1H, t), 7.1(1H, s), 4.2(2H, d), 3.6(4H, br), 3.5(4H, br). | 360.30 |

### Example #110

### 2,3-dichloro-5-{N-[(1,1-dioxothiomorpholino)carbonylmethyl]carbamoyl}-4H-thieno[3,2b]-pyrrole and

### Example #111

### 2,3-dichloro-5-{N-(1-oxothiomorpholino)carbonylmethyl]carbamoyl}-4H-thieno[3,2b]pyrrole

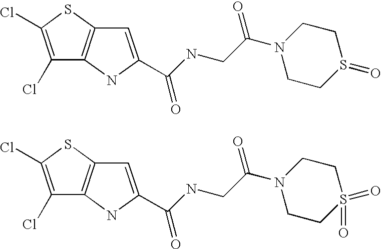

A solution of m-chloroperbenzoic acid (mCPBA) (14 mg, 0.85 mmol) in dichloromethane (5 ml) was added dropwise to a suspension of 2,3-dichloro-5-[N-(thiomorpholinocarbonylmethyl)carbamoyl]-4H-thieno[3,2-b]pyrrole (example #108) in dichloromethane and the reaction mixture stirred at ambient temperature for one hour. 5% sodium metabisulfite solution (5 ml) was added and the mixture stirred for several minutes. The aqueous layer was extracted with ethyl acetate (2×15 ml) and the combined organic extracts washed with sodium bicarbonate solution (2×15 ml) and concentrated. The two component mixture was separated using bond-elute silica column chromatography (eluent: dichloromethane-dichloromethane/methanol 5% gradient) to afford the less polar product (sulfone) as a white powder (57 mg 33%) and the more polar product (sulfoxide) as a white solid (62 mg, 37%).

NMR: (sulfone) 12.43 (1H, br), 8.4 (1H, t), 7.1 (1H, s), 4.2 (2H, d), 3.9 (4H, br), 3.3 (2H, br), 3.1 (2H, br); m/z 408.33

NMR: (sulfoxide) 12.42 (1H, br), 8.4 (1H, t), 7.1 (1H, s), 4.2 (3H, m), 3.9 (2H, d), 3.6 (1H, m), 2.9 (4H, m).

### Example #112

### 2-Chloro-5-[N-(2-indanyl)carbamoyl]-6H-thieno[2,3-b]pyrrole

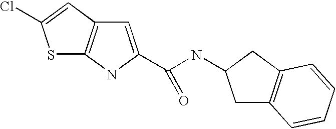

5-Carboxy-2-chloro-6H-thieno[2,3-b]pyrrole (Method #10; 101 mg, 0.5 mmol) was dissolved in dichloromethane (6 ml) containing 2-aminoindane (68 mg, 0.5 mmol), 1-Hydroxybenotriazole (HOBT) (68 mg, 0.5 mmol) and DIPEA (355 μl, 2.0 mmol). The mixture was stirred for one minute before the addition of EDAC (125 mg, 0.65 mmol). The mixture was stirred at ambient temperature for approximately 16 hours before being washed with water. The organic fraction was concentrated and was purified using bon-elute silica column chromatography (eluent: dichloromethane-dichloromethane/methanol 2.5% gradient) to afford the title compound as a beige solid (96 mg, 61%).

NMR: 11.80 (1H, br), 8.3 (1H, t), 7.2 (5H, m), 7.0 (1H, s), 4.6 (1H, m), 4.7 (1H, d), 3,2 (2H, m), 2.9 (2H, m); m/z 315.46

### Example #113

### 5-[N-(Benz[1,2]oxazol-3-ylmethyl)carbamoyl]-2,3-dichloro-4H-thieno[3,2-b]pyrrole

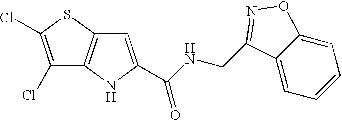

5-Carboxy-2,3-dichloro-4H-thieno[3,2-b]pyrrole (Method #9; 118 mg, 0.5 mmol) was dissolved in DMF (5 ml) containing HOBT (83 mg, 0.55 mmol), DIPEA (52 ul, 0.30 mmol) and 1,2-Benzisoxazole-3-methylamine (Eur. J. Med. Chem-Chimica Therapeutica, January-February-10, No.1 p32) (89 mg, 0.6 mmol). The mixture was stirred for one minute and EDAC (106 mg, 0.55 mmol) was added. The mixture was stirred at room temperature for approximately 18 hours. Water was added to the solution and a solid precipitated out. This solid was filtered off and was washed with water before being dried under reduced pressure to afford the title compound as a white solid (174 mg) 1H NMR: 4.9 (2H, s), 7.1 (1H, s), 7.4 (1H, t), 7.6 (1H, t), (7.8 (1H, d), 8.0 (1H, d), 9.2 (1H, s), 12.5 (1H, s); m/z 366 (M+H);

HPLC Hichrome C18 column Acetonitrile/water/0.1%TFA 5-95% over 7.5 min Rt 4.9 min m/z 366 (M+H)

The following compounds were made by the process of Example #113 using 5-carboxy-2,3-dichloro-4H-thieno[3,2-b]pyrrole (Method #9) and the appropriate amine:

### Example #114

### 2,3-Dichloro-5-(N-{2-[2-(hydroxymethyl)phenyl]ethyl}carbamoyl)-4H-thieno[3,2-b]pyrrole

### Example #115

### 2,3-Dichloro-5-[N-(4-phenylisoxazol-3-ylmethyl)carbamoyl]-4H-thieno[3,2-b]pyrrole

### Example #116

### 2,3-Dichloro-5-(N-{2-[2-(2-morpholinoethoxy)phenyl]ethyl}carbamoyl)-4H-thieno[3,2-b]pyrrole

### Example #117

### 2,3-Dichloro-5-(N-{2-[2-(methoxycarbonylmethoxy)phenyl]ethyl}-carbamoyl)-4H-thieno[3,2-b]pyrrole

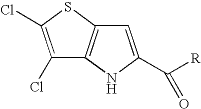

| Ex | R | NMR | Rt | M/z |
|---|---|---|---|---|
| #114¹ | 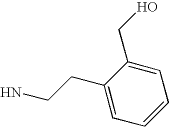 | 1H NMR: 2.9(2H, q), 3.4(2H, q), 4.6(2H, d), 5.1(1H, t), 7.1(1H, s), 7.2(3H, m), 7.4(1H, m), 8.4(1H, m) | 4.55 | 369 |
| #115² | 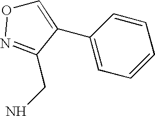 | 1H NMR: 4.7(2H, s), 7.1(1H, s), 7.4(3H, m), 7.6(2H, m), 8.8(1H, t), 9.2(1H, s) | 5.01 | 392 |
| #116³ | 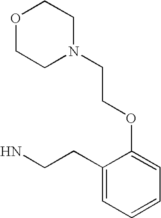 | 1H NMR(CDCl3): 2.6(2H, m), 2.8(2H, t), 3.0(2H, t), 3.7(6H, m), 4.1(2H, t), 6.3(1H, m), 6.6(1H, s), 6.9(2H, m), 7.2(2H, m), 9.8(1H, b) | 4.26 | 468 |
| #117⁴ | 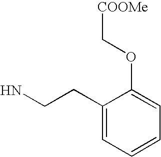 | (CDCl3): 3.0(2H, q), 3.8(2H, q, 3.9(3H, s), 4.7(2H, s), 6.6(1H, s), 6.7(1H, s), 6.8(1H, d), 7.0(1H, t), 7.2(2H, m) | 4.47 | 413 |

¹Amine: EP86-300884
²Amine: Method #33
³Amine: Method #34
⁴Amine: Method #35

### Example #118

### 5-(N-{2-[2-(Carboxymethoxy)phenyl]ethyl}carbamoyl)-2,3-dichloro-4H-thieno[3,2-b]pyrrole

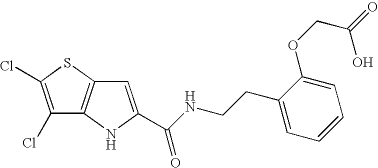

2,3-Dichloro-5-(N-{2-[2-(methoxycarbonylmethoxy)phenyl]ethyl}carbamoyl)-4H-thieno[3,2-b]pyrrole (Example #117, 100 mg, 0.25 mmol) dissolved in 2:1 THF: methanol (2 ml), was treated with 1N Lithium hydroxide solution (0.25 ml, 0.25 mmol), followed by the addition of water till the solution was just opalescent and then stirred for 2 hours at room temperature. The organic solvents were removed by evaporation under reduced pressure, the solution filtered then acidified with 2N HCl to give a thick white precipitate which was isolated by filtration, washed with water and dried under reduced pressure over phosphorous pentoxide to give the title compound (83 mg)

¹H NMR: 2.9 (2H, q), 3.5 (2H, q), 4.7 (2H, s), 6.8 (2H, m), 7.1 (1H, s), 7.2 (2H, m), 8.4 (1H, m), 12.4 (1H, s)

HPLC Hichrome C18 column Acetonitrile/water/0.1%TFA 5-95% over 7.5 min Rt 4.47 min m/z 413 (M+H)

### Example #119

### 2,3-Dichloro-5-{N-[2-(3-methoxyphenyl)ethyl]carbamoyl}-4H-thieno[3,2-b]pyrrole

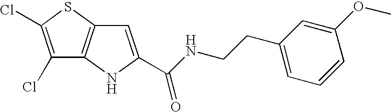

5-Carboxy-2,3-dichloro-4H-thieno[3,2-b]pyrrole (Method #9; 100 mg, 0.42 mmol) was suspended in DCM 10 ml; 2M oxalyl chloride solution in DCM (750 ul, 1.5 mmol) was added dropwise followed by one drop of DMF and the resultant mixture stirred overnight at room temperature. The suspension was filtered, the residue washed with DCM and the filtrate evaporated to dryness under reduced pressure and then azeotroped with toluene to give a yellow solid which was dissolved in DCM (6.9 ml) under nitrogen. Calcium carbonate (60 mg, 0.6 mmol) was added followed by a solution of 3-methoxyphenethylamine; 90 mg, 0.4 mmol) and the mixture was stirred at room temperature overnight. The mixture was filtered, the residue washed with DCM followed by 0.1 M HCl then water to give the title compound as a white solid (98 mg) ¹H NMR (CDCl3): 2.9 (2H, t), 3.7 (2H, t), 3.9 (3H, s), 6.0 (1H, m), 6.6 (1H, s), 6.8 (3H, m), 7.2 (2H, m), 9.9 (1H, s)

m/z 369 (M+H)

### Example #120

### 2,3-Dichloro-5-[N-(2-oxo-1,2,3,4-tetrahydroquinol-3-yl)carbamoyl]-4H-thieno[3,2-b]pyrrole

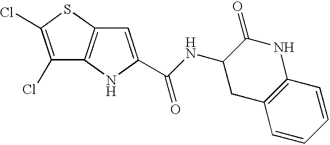

This was prepared following the procedure described in Example #97 using 3-aminohydrocarbostyril (Arch. Biochem & Biophys. 1963 109 48) was used in place of 4-aminobenzyl alcohol and 5-carboxy-2,3-dichloro-4H-thieno[3,2-b]pyrrole (Method #9) was used in place of 5-(N-carboxymethylcarbamoyl)-2,3-dichloro-4H-thieno[3,2-b]pyrrole to give the title compound: 1H NMR: 3.0 (2H, d), 4.7 (1H, q), 6.9 (3H, m), 7.2 (2H, m), 8.5 (1H, d), 10.35 (1H, s)

HPLC Hichrome C18 column Acetonitrile/water/0.1%TFA 5-95% over 7.5 min Rt 4.44 min m/z 380 (M+H)

### Example #121

### 2,3-Dichloro-5-(N-{2-[2-(2-methoxyethoxy)phenyl]ethyl}carbamoyl)-4H-thieno[3,2-b]pyrrole

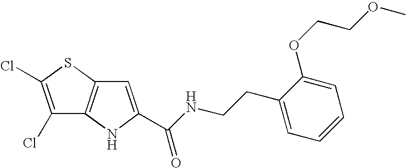

2,3-Dichloro-5-{N-[2-(2-hydroxyphenyl)ethyl]carbamoyl}-4H-thieno[3,2-b]pyrrole (Example 73, 177.5 mg, 0.5 mmol) was dissolved in THF 3 ml under nitrogen together with methoxyethanol (38 μl, 0.5 mmol) and triphenylphosphine (131 mg, 0.5 mmol), the resultant stirred solution was cooled to 0C and treated with DIAD (98 μl, 0.5 mmol) dropwise over 30 mins, then allowed to warm to room temperature overnight. After evaporation to dryness the mixture was purified by chromatography on a 20 g Bond Elute silica column eluting with DCM. The product was taken up in diethyl ether 20 ml, washed with 2N NaOH (3×5 ml), water (5 ml) and saturated brine (5 ml) then dried over magnesium sulphate and evaporated under reduced pressure to give the title compound (45 mg) ¹H NMR (CDCl3): 3.0 (2H, t), 3.5 (3H, s), 3.7 (2H, m), 3.9 (2H, m), 4.2 (2H, m), 6.6 (1H, m), 6.8 (1H, s), 6.9 (2H, m), 7.2 (2H, m), 9.9 (1H, s) HPLC Hichrome C18 column Acetonitrile/water/0.1%TFA 5-95% over 7.5 min Rt 5.02 min m/z 411 (M−H)

### Example #122

### 5-(N-{2-[2-(Carbamoylmethoxy)phenyl]ethyl}carbamoyl)-2,3-dichloro-4H-thieno[3,2-b]pyrrole

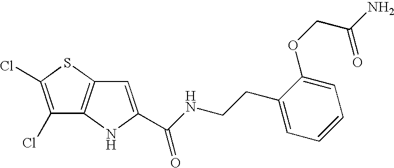

5-(N-{2-[2-(Carboxymethoxy)phenyl]ethyl}carbamoyl)-2,3-dichloro-4H-thieno[3,2-b]pyrrole (Example #118, 124 mg, 0.3 mmol) was dissolved in DMF (3 ml) containing HOBT (50 mg, 0.33 mmol), DIPEA (140 μl, 0.69 mmol) and ammonium chloride (18 mg, 0.36 mmol). The mixture was stirred for one minute and EDAC (64 mg, 0.33 mmol) was added. The mixture was stirred at room temperature for approximately 18 hours. Water was added to the solution and a solid precipitated out. This solid was filtered off and was washed with water before being dried under reduced pressure to afford the title compound as a white solid (111 mg) 1H NMR: 2.9 (2H, q), 3.5 (2H, q), 4.5 (2H, s), 6.8 (2H, m), 7.0 (1H, s), 7.2 (2H, m), 7.5 (1H, s), 7.6 (1H, s), 8.4 (1H, m), 12.4 (1H, s)

HPLC Hichrome C18 column Acetonitrile/water/0.1%TFA 5-95% over 7.5 min Rt 4.37 min m/z 412 (M+H)

The following compounds were made by the process of Example #122 using 5-(N-{2-[2-(Carboxymethoxy)phenyl]ethyl}carbamoyl)-2,3-dichloro-4H-thieno[3,2-b]pyrrole (Example #118) and the appropriate amine:

### Example #123

### 2,3-Dichloro-5-(N-{2-[2-(N-methylcarbamoylmethoxy)phenyl]ethyl}-carbamoyl)-4H-thieno[3,2-b]pyrrole

### Example #124

### 2,3-Dichloro-5-(N-{2-[2-(N,N-dimethylcarbamoylmethoxy)phenyl]-ethyl}carbamoyl)-4H-thieno[3,2-b]pyrrole

### Example #125

### 2,3-Dichloro-5-(N-{2-[2-(morpholinocarbonylmethoxy)phenyl]ethyl}-carbamoyl)-4H-thieno[3,2-b]pyrrole

### Example #126

### 5-(N-{2-[2-(N-Benzylcarbamoylmethoxy)phenyl]ethyl}carbamoyl)-2,3-dichloro-4H-thieno[3,2-b]pyrrole

### Example #127

### 2,3-Dichloro-5-(N-{2-[2-(4-hydroxypiperidinocarbonylmethoxy)phenyl]-ethyl}carbamoyl)-4H-thieno[3,2-b]pyrrole

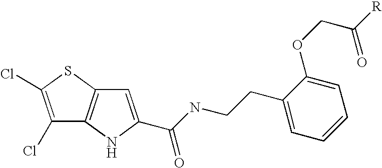

| Ex | R | NMR | Rt | M/z |
|---|---|---|---|---|
| #123 | 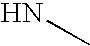 | 1H NMR: 2.8(3H, d), 2.9(2H, q), 3.5(2H, q), 4.5(2H, s), 6.8(2H, m), 7.1(1H, s), 7.2(2H, m), 8.0(1H, m), 8.5(1H, m), 12,2(1H, s) | 4.65 | 426 |
| #124 | 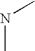 | 1H NMR: 2.9(5H, m), 3.0(3H, s), 3.5(2H, q), 4.8(2H, s), 6.8(2H, m), 7.0(1H, s), 7.1(2H, m), 8.4(1H, m), 12.4(1H, s) | 4.57 | 440 |
| #125 | 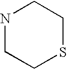 | 1H NMR: 2.9(2H, q), 3.4-3.7(10H, m), 4.8(2H, s), 6.8(2H, m), 7.0(1H, s), 7.1(2H, m), 8.3(1H, m), 12.4(1H, s) | 4.5 | 484 |
| #126 | 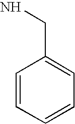 | 1H NMR: 2.9(2H, q), 3.5(2H, q), 4.4(2H, d), 4.6(2H, s), 6.9(2H, m), 7.0(1H, s), 7.2-7.4(7H, m), 8.4(1H, m), 8.6(1H, m), 12.4(1H, s) | 5.26 | 502 |
| #127 | 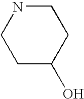 | 1H NMR: 1.2-1.5(2H, m), 1.6-1.8(2H, m), 2.9(2H, q), 3.1(1H, q), 3.2(1H, q), 3.5(2H, q), 3.7(2H, q), 3.9(1H, m), 4.7(1H, d), 4.8(2H, s), 6.9(2H, m), 7.1(1H, s), 7.2(2H, m), 8.0(1H, s), 8.4(1H, m), 12.4(1H, s) | 4.2 | 496 |

### Example #128

### (S)-2-Chloro-5-{N-[(α-(5-ethoxycarbonyl-1,3,4-oxadiazol-2-yl)phenethyl]carbamoyl}-6H-thieno[2,3-b]pyrrole

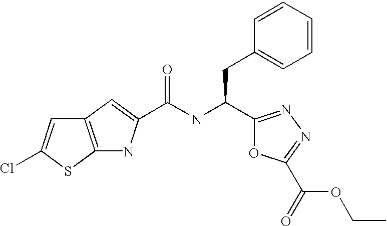

5-Carboxy-2-chloro-6H-thieno[2,3-b]pyrrole (Method #10 172 mg, 0.86 mmol) was dissolved in DCM (10 ml) containing HOBT (115 mg, 0.86 mmol), DIPEA (331 mg, 2.57 mmol) and ethyl (S)-5-(α-aminophenethyl)-1,3,4-oxadiazole-2-carboxylate trifluoroacetate (Method #16; 322 mg, 0.86 mmol). EDAC (205 mg, 1.07 mmol) was added and the mixture stirred at ambient temperature for 16 hours. The reaction mixture was filtered and the filtrate washed with dilute hydrochloric acid and water. After drying over magnesium sulphate and concentration the crude material was purified by bond elute silica column chromatography (eluent—DCM/Ethyl acetate gradient 0-20%) to give the title compound (104 mg, 27%) NMR 1.3 (3H, t); 3.3-3.5 (2H, m); 4.4 (2H, q); 5.5-5.6 (1H, m); 7.05 (1H, s); 7.15 (1H, s); 7.2-7.35 (5H, m); 8.95 (1H, d); 11.84 (1H, s)

m/z 444.9

### Example #129

(S)-2-chloro-5-{N-[(α-(4-methoxycarbonyloxazol-5-yl)phenethyl]carbamoyl}-6H-thieno[2,3-b]pyrrole

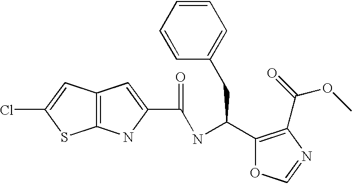

5-Carboxy-2-chloro-6H-thieno[2,3-b]pyrrole (Method #10, 100 mg, 0.5 mmol) was dissolved in DCM (10 ml) containing HOBT (68 mg, 0.5 mmol), DIPEA (193 mg, 1.5 mmol) and methyl (S)-5-(α-aminophenethyl)oxazole-4-carboxylate trifluoroacetate (Method #32, 230 mg, 0.5 mmol). EDAC (143 mg, 0.75 mmol) was added and the mixture stirred at ambient temperature for 4 hours. The reaction mixture was diluted with ethyl acetate (75 ml) washed with dilute citric acid, water and brine, dried over magnesium sulphate and concentrated. The crude material was purified by bond elute silica column chromatography (eluent—DCM/Ethyl acetate gradient 0-50%) to give the title compound (147 mg, 34%).

NMR 3.05-3.15 (1H, m); 3.2-3.25 (1H, m); 3.8 (3H, s); 5.85-5.95 (1H, m); 7.1 (1H, s); 7.15 (1H, s); 7.15-7.25 (5H, m); 7.75 (1H, d) 8.4 (1H, s); 11.74 (1H, s); m/z 429

### Example #130

### 2-Chloro-5-{N-[α-(3-pyridyl)phenethyl]carbamoyl}-6H-thieno[2,3-b]pyrrole

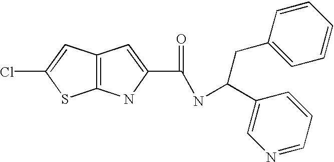

5-Carboxy-2-chloro-6H-thieno[2,3-b] pyrrole (Method #10, 100 mg, 0.5 mmol) was dissolved in DCM (10 ml) containing HOBT (68 mg, 0.5 mmol), DIPEA (193 mg, 1.5 mmol) and α-(3-pyridyl)phenethylamine dihydrochloride (J. Am. Chem. Soc., 1950, 72, 1988; 135 mg, 0.5 mmol). EDAC (143 mg, 0.75 mmol) was added and the mixture stirred at ambient temperature for 16 hours. The reaction mixture was diluted with ethyl acetate (50 ml), water (20 ml) was added and the pH adjusted to 7 with dilute hydrochloric acid. The organic fraction was separated washed with water and brine, dried over magnesium sulphate and concentrated. The crude material was purified by bond elute silica column chromatography (eluent—Ethyl acetate) to give the title compound as a solid (128 mg, 67%).

NMR 3.0-3.2 (2H, m); 5.2-5.3 (1H, m); 7.05 (1H, s); 7.1-7.2 (2H, m); 7.2-7.4 (5H, m); 7.8 (1H, d); 8.4 (1H, d); 8.55-8.65 (2H, m); 11.71 (1H, s); m/z 381

### Example #131

### 2,3-dichloro-5-{N-[α-(3-pyridyl)phenethyl)]carbamoyl}-4H-thieno[3,2-b]pyrrole

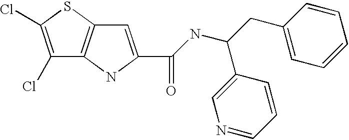

5-carboxy-2,3-dichloro-4H-thieno[3,2-b]pyrrole (Method #9, 118 mg, 0.5 mmol) was dissolved in DCM (10 ml) containing HOBT (68 mg, 0.5 mmol), DIPEA (193 mg, 1.5 mmol), α-(3-pyridyl)phenethyl)amine dihydrochloride (J. Am. Chem. Soc., 1950, 72, 1988; 135 mg, 0.5 mmol). EDAC (143 mg, 0.75 mmol) was added and the mixture stirred at ambient temperature for 16 hours. The reaction mixture was diluted with ethyl acetate (100 ml), water (20 ml) was added and the pH adjusted to 7 with dilute hydrochloric acid. The organic fraction was separated washed with water and brine, dried over magnesium sulphate and concentrated. The crude material was purified by bond elute silica column chromatography (eluent—Ethyl acetate) to give the title compound as a solid (72 mg, 34%)

NMR 3.0-3.3 (2H, m); 5.2-5.3 (1H, m); 7.1-7.2 (2H, m); 7.2-7.4 (5H, m); 7.85 (1H, dt); 8.4 (1H, dd); 8.6 (1H, s); 8.75 (1H, d); 12.28 (1H, s); m/z 417

### Example #132

### (S)-2-Chloro-5-{N-[(α-(3-phenyl-1,2,4-oxadiazol-5-yl)phenethyl]carbamoyl}-6H-thieno[2,3-b]pyrrole

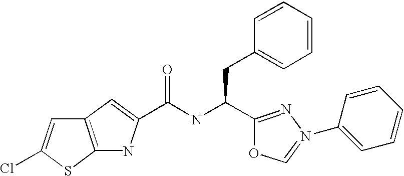

5-Carboxy-2-chloro-6H-thieno[2,3-b] pyrrole (Method #10, 201 mg, 1 mmol) was dissolved in DCM (15 ml) containing HOBT (148 mg, 1.1 mmol), DIPEA (387 mg, 3 mmol) and (S)-5 (α-aminophenethyl)-3-phenyl-1,2,4-oxadiazole trifluoroacetate (Method #18, 379 mg, 1 mmol). EDAC (238 mg, 1.25 mmol) was added and the mixture stirred at ambient temperature for 16 hours. The reaction mixture was filtered and the filtrate diluted with DCM (50 ml) washed with dilute hydrochloric acid and water. After drying over magnesium sulphate and concentration the crude material was purified by bond elute silica column chromatography (eluent—DCM/Ethyl acetate gradient 0-20%) to give the title compound (120 mg, 26%); NMR 3.3-3.6 (2H, m); 5.5-5.7 (1H, m); 7.1 (1H, s); 7.15-7.4 (6H, m); 7.55-7.65 (3H, m); 8.05 (2H, d); 9.05 (1H, d); 11.9 (1H, s); m/z 449

### Example #133

### 2,3-Dichloro-5-[N-(1-hydroxyindan-2-yl)carbamoyl]-4H-thieno[3,2-b]pyrrole

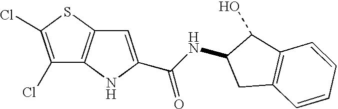

5-Carboxy-2,3-dichloro-4H-thieno[3,2-b]pyrrole (Method #9, 329 mg, 1.4 mmol) was dissolved in DCM (20 ml) containing HOBT (189 mg, 1.4 mmol), DIPEA (0.5 ml, 2.8 mmol) and 2-aminoindan-1-ol (Method #36, 250 mg, 1.4 mmol). The reaction mixture was stirred for one minute and EDAC (306 mg, 1.6 mmol) was added. The reaction mixture was stirred at room temperature for approximately 18 hours. The resulting solution was washed with water (20 ml) and the aqueous layer extracted with DCM (2×20 ml). The organic extracts were combined, dried over magnesium sulphate and concentrated under reduced pressure to give the title compound as a white solid (70 mg, 14%).

NMR: 2.8 (1H, dd), 3,2 (1H, dd), 4.4 (1H, quin), 5.1 (1H, d), 7.1 (1H, s), 7.2-7.4 (4H, m), 8.7 (1H, d), 12.4 (1H, s); m/z 366 (M−H)

### Example #134

### 2,3-Dichloro-5-[N-((1S,2S)-1-hydroxyindan-2-yl)carbamoyl]-4H-thieno[3,2-b]pyrrole

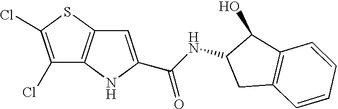

and

### Example #135

### 2,3-Dichloro-5-[N-((1R,2R)-1-hydroxyindan-2-yl)carbamoyl]-4H-thieno[3,2-b]pyrrole

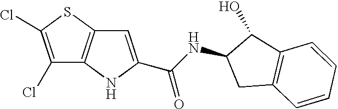

2,3-Dichloro-5-[N-(1-hydroxyindan-2-yl)carbamoyl]-4H-thieno[3,2-b]pyrrole (Example #133) was subject to preparative HPLC under the following conditions to give 2,3-dichloro-5-[N-((1S,2S)-1-hydroxyindan-2-yl)carbamoyl]-4H-thieno[3,2-b]pyrrole as a white solid (9 mg) and 2,3-dichloro-5-[N-((1R,2R)-1-hydroxyindan-2-yl)carbamoyl]-4H-thieno[3,2-b]pyrrole as a white solid (12 mg).

| | |
|---|---|
| Instrument | P.E Series 200 system 1 |
| Column | Chiralpak AD (250 mm × 4.6 mm) |
| | No.ADOOCE-AJ052 |
| Eluent | MeOH |
| Oven Temperature | Ambient |
| Flow | 1 ml/min |
| Wavelength | 254 nm |
| Sample concentration | 1 mg/ml in EtOH + sonication |

### Example #136

### 2-Chloro-5-[1-(1-hydroxyindan-2-yl)carbamoyl]-6H-thieno[2,3-b]pyrrole

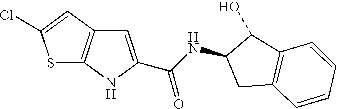

5-Carboxy-2-chloro-6H-thieno[2,3-b]pyrrole (Method #10; 280 mg, 1.4 mmol) was dissolved in DCM (20 ml) containing HOBT (189 mg, 1.4 mmol), DIPEA (0.5 ml, 2.8 mmol) and 2-aminoindan-1-ol (Method #36, 250 mg, 1.4 mmol). The reaction mixture was stirred for one minute and EDAC (306 mg, 1.6 mmol) was added. The reaction mixture was stirred at room temperature for approximately 18 hours. The resulting solution was washed with water (20 ml) and the aqueous layer extracted with DCM (2×20 ml). The organic extracts were combined, dried over magnesium sulphate and concentrated under reduced pressure to give the title compound as a white solid (78 mg, 17%).

NMR: 2.8 (1H, dd), 3.2 (1H, dd), 4.4 (1H, quin), 5.1 (1H, t), 5.6 (1H, d), 7.1 (1H, s), 7.2-7.4 (5H, m), 8.4 (1H, d), 11.8 (1H, s); m/z 331 (M−H)

### Example #137

### 2,3-Dichloro-5-[N-(1-hydroxy-1,2,3,4-tetrahydronaphth-2-yl)carbamoyl]-4H-thieno[3,2-b]pyrrole

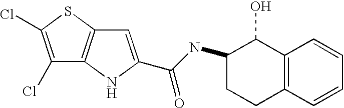

5-Carboxy-2,3-dichloro-4H-thieno[3,2-b]pyrrole (Method #9; 216 mg, 0.9 mmol) was dissolved in DCM (15 ml) containing HOBT (122 mg, 0.9 mmol), DIPEA (0.3 ml, 1.8 mmol) and 2-amino-1,2,3,4-tetrahydronaphth-1-ol (Method #39, 150 mg, 0.9 mmol). The reaction mixture was stirred for one minute and EDAC (206 mg, 1.1 mmol) was added. The reaction mixture was stirred at room temperature for approximately 18 hours. The resulting solution was washed with water (20 ml) and the aqueous layer extracted with DCM (2×20 ml). The organic fractions were combined and concentrated under reduced pressure to give the title compound as a white solid (70 mg, 14%). NMR 1.8 (1H, m), 2.0 (1H, qd), 2.9 (1H, m), 3.3 (1H, qd), 4.4 (1H, quin), 4.9 (1H, m), 5.2 (1H, s), 5.8 (1H, brs), 6.8 (1H, s), 7.0 (1H, dd), 7.1 (1H, d), 7.2 (1H, s), 7.5 (1H, d) 10.1 (1H, brs). m/Z 380 (M−H).

### Example #138

### 2,3-Dichloro-5-[N-(6-fluoro-1-hydroxyindan-2-yl)carbamoyl]-4H-thieno[3,2-b]pyrrole

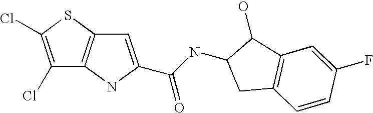

5-Carboxy-2,3-dichloro-4H-thieno[3,2-b]pyrrole (Method #9; 141 mg, 0.6 mmol) was dissolved in DCM (10 ml) containing HOBT (81 mg, 0.6 mmol), DIPEA (0.2 ml, 1.2 mmol) and 2-amino-6-fluoro-1-indanol (Method #38, 100 mg, 0.6 mmol). The reaction mixture was stiffed for one minute and EDAC (138 mg, 0.7 mmol) was added. The reaction mixture was stirred at room temperature for approximately 18 hours. The resulting solution was washed with water (20 ml) and the aqueous layer extracted with DCM (2×20 ml). The organic fractions were combined and concentrated under reduced pressure to give a white solid. Purification by flash column chromatography (Isohexane:ethyl acetate 1:1) gave the title compound as a white solid (70 mg, 14%). NMR 3.0 (1H, dd), 3.3 (1H, m), 4.6 (1H, q), 4.9 (1H, t), 5.5 (1H, d), 4.9 (1H, m), 6.9-7.2 (4H, m), 8.1 (1H, d), 12.4 (1H, brs); m/Z 383 (M−H)

### Example #139

### 2,3-Dichloro-5-[N-(7-methoxy-1-oxo-1,2,3,4-tetrahydronaphth-2-yl)carbamoyl]-4H-thieno[3,2-b]pyrrole

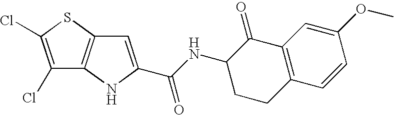

5-Carboxy-2,3-dichloro-4H-thieno[3,2-b]pyrrole (Method #9; 216 mg, 0.9 mmol) was dissolved in DCM (20 ml) containing HOBT (122 mg, 0.9 mmol), DIPEA (0.3 ml, 1.8 mmol) and 2-amino-7-methoxy-1-oxo-1,2,3,4-tetrahydronaphthalene ((Farmaco, Ed. Sci.(1985), 40 (6), 422-428), 204 mg, 0.9 mmol). The reaction mixture was stirred for one minute and EDAC (206 mg, 1.0 mmol) was added. The reaction mixture was stirred at room temperature for approximately 18 hours. The resulting solution was washed with water (20 ml) and the aqueous layer extracted with DCM (2×20 ml). The organic fractions were combined and concentrated under reduced pressure to give the title compound as a brown solid (100 mg, 27%). NMR 3.0 (1H, dd), 3.1 (1H, dd), 3.3 (2H, m), 4.8 (1H, q), 5.8 (1H, s), 6.9-7.2 (4H, m), 8.6 (1H, d), 12.2 (1H, brs); m/z 408 (M−H).

### Example #140

### 2,3-Dichloro-5-[N-(2-indanyl)carbamoyl]-4H-thieno[3,2-b]pyrrole

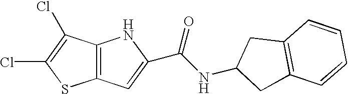

5-Carboxy-2,3-dichloro-4H-thieno[3,2-b]pyrrole (Method #9, 45 mg, 0.19 mmol) was dissolved in DMF (10 ml) with 2-aminoindan (28 mg, 0.21 mmol), di-isopropylethylamine (0.1 ml, 0.57 mmol) and HATU (80 mg, 0.21 mmol). The mixtures were stirred at ambient temperature for approximately 16 hours. The mixture was partitioned between water and ethyl acetate and was washed with water (×5). The aqueous phase was dried, filtered and concentrated, and the residue purified by silica bond-elut chromatography using a gradient of ethyl acetate in iso-hexane (0-50%) as eluent to give the title compound, m/z 351.

Following a similar procedure to the process of Example #140 the following examples were prepared:

### Example #141

### 2,3-Dichloro-5-[N-(3-methylisoxazol-5-yl)methyl]carbamoyl]-4H-thieno[3,2-b]pyrrole

### Example #142

### 2,3-Dichloro-5-[N-(4-hydroxy-1,1-dioxotetrahydrothiophen-3-yl)carbamoyl]-4H-thieno[3,2-b]pyrrole

### Example #143

### 2,3-dichloro-5-(N-{N-methyl-N-[(1-oxo-1,2,3,4-tetrahydronaphth-2-yl)methyl]carbamoylmethyl}carbamoyl)-4H-thieno[3,2-b]pyrrole

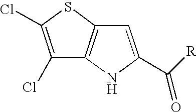

| Example | R | M/z |
|---|---|---|
| #141 | 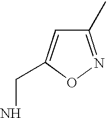 | 329 |
| #142 | 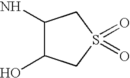 | 365 |
| #143¹ | 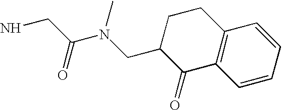 | 491 |

¹Amine. Method #37

### Example #144

### 2-Chloro-5-[N-(2-oxo-1,2,3,4-tetrahydroquinol-3-yl)carbamoyl]-6H-thieno[2,3-b]pyrrole

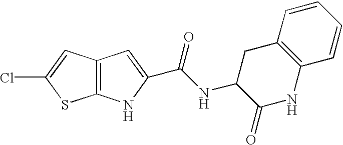

5-Carboxy-2-chloro-6H-thieno[2,3-b]pyrrole (Method #10; 101 mg, 0.5 mmol) was dissolved in DMF (2.5 ml) containing 3-amino-3,4-dihydro-2 (1H)-quinolinone [J Med Chem (1986) 29 (12) 2427-32] (99 mg, 0.5 mmol), HOBT (68 mg, 0.5 mmol) and Et₃N (55 mg, 0.5 mmol). The mixture was stirred for 1 minute before the addition of EDAC (96 mg, 0.5 mmol). The mixture was stirred at ambient temperature for approximately 18 hours before being poured into water (50 ml), stirred vigorously and filtered. The recovered solid was washed with water, ether and dried to give the title compound as a amorphous solid.(161 mg). NMR (DMSOd₆): 11.96 (1H, s), 10.36 (1H, s), 8.50 (1H, d), 7.20 (2H, m), 7.19 (1H, s), 7.09 (1H, s), 6.96 (1H, m), 6.91 (1H, m), 4.72 (1H, m), 3.08 (2H, m); MH⁺ 346.14.

### Example #145

### 2-Chloro-5-[N-(1,2,3,4-tetrahydroquinol-3-yl)carbamoyl]-6H-thieno[2,3-b]pyrrole

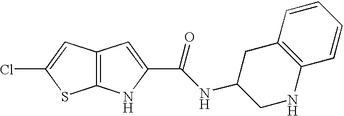

5-Carboxy-2-chloro-6H-thieno[2,3-h]pyrrole (Method #10; 157 mg, 0.78 mmol) was dissolved in DMF (4 ml) containing 3-amino-1,2,3,4-tetrahydroquinoline [J Med Chem (1982) 25 (1) 68-70] (115 mg, 0.78 mmol) and HOBT (105 mg, 0.78 mmol). The mixture was stirred for 1 minute before the addition of EDAC (149 mg, 0.78 mmol). The mixture was stirred at ambient temperature for approximately 64 hours before being partitioned between water and EtOAc. The organics were washed with water, saturated aqueous NaHCO₃, water, saturated brine and dried. The organics were filtered, concentrated and chromatographed on Fluorochem silica 40-63μ 60A (eluent 40:60 EtOAc/isohexane) to afford the title compound as an amorphous solid (44 mg). NMR (DMSOd₆): 11.94 (1H, s), 8.04 (1H, d), 7.16 (1H, s), 7.06 (1H, s), 6.90 (2H, m), 6.48 (2H, m), 5.8 (1H, br), 4.18 (1H, m), 3.05 (1H, t), 2.85 (2H, in); MH⁺ 332.17.

### Example #146

### 2-Chloro-5-[N-(1-methyl-2-oxo-1,2,3,4-tetrahydroquinol-3-yl)carbamoyl]-6H-thieno[2,3-b]pyrrole

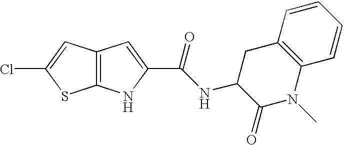

5-Carboxy-2-chloro-6H-thieno[2,3-b]pyrrole (Method #10; 80 mg, 0.4 mmol) was dissolved in DMF (2 ml) containing 3-amino-3,4-dihydro-1-carbostyril [JCS 1965 1080-1087] (71 mg, 0.4 mmol) and HOBT (54 mg, 0.4 mmol). The mixture was stirred for 1 minute before the addition of EDAC (77 mg, 0.4 mmol). The mixture was stirred at ambient temperature for approximately 18 hours before being partitioned between water and EtOAc. The organics were washed with water, saturated aqueous NaHCO₃, water, saturated brine and dried. The organics were filtered, concentrated and recrystalised from EtOAc to afford the title compound as an amorphous solid (66 mg). NMR 11.96 (1H, s), 8.54 (1H, d), 7.30 (2H, m), 7.17 (2H, m), 7.08 (2H, m), 4.68 (1H, m), 3.32 (3H, s), 3.14 (1H, m), 3.04 (1H, m); m/z (MH⁺) 360.14

### Example #147

### 2-Chloro-5-[N-(3-oxo-2,3,4,5-tetrahydro-1H-benz[2]azepin-4-yl)carbamoyl]-6H-thieno[2,3-b]pyrrole

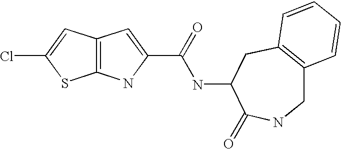

5-Carboxy-2-chloro-6H-thieno[2,3-b]pyrrole (Method #10; 101 mg, 0.5 mmol) was dissolved in DMF (2.5 ml) containing 4-amino-1,2,4,5-tetrahydro-3H-2-benzazepin-3-one hydrochloride [CAS Reg No 148842-85-7] (107 mg, 0.5 mmol), HOBT (68 mg, 0.5 mmol) and Et₃N (101 mg, 1.0 mmol). The mixture was stirred for 1 minute before the addition of EDAC (96 mg, 0.5 mmol), then at ambient temperature for approximately 18 hours before being partitioned between water and EtOAc. The organics were washed with water, saturated aqueous NaHCO₃, water, saturated brine and dried; filtered and evaporated to afford the title compound as an amorphous solid (26 mg). NMR: 11.92 (1H, s), 8.33 (1H, t), 8.29 (1H, d), 7.2 (6H, m), 5.30 (1H, m), 4.83 (1H, dd), 3.98 (1H, dd), 3.20 (2H, m); m/z (MH⁺) 360.19.

### Example #148

### 2,3-Dichloro-5-[N-(1-methoxyindan-2-yl)carbamoyl]-4H-thieno[3,2-b]pyrrole

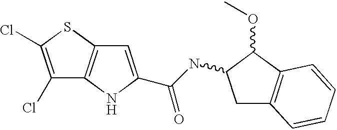

5-Carboxy-2,3-dichloro-4H-thieno[3,2-b]pyrrole (Method #9, 145 mg, 0.613 mmol), trans-2-amino-1-methoxyindan (Method #40, 100 mg, 0.163 mmol), DIPEA (0.105 ml, 0.613 mmol), and HOBT (83 mg, 0.613 mmol) was stirred in dichloromethane (5 ml) for one minute. EDAC (147 mg, 0.766 mmol) was added and the mixture stirred at room temperature for 20 hours. The reaction mixture was evaporated, ethyl acetate (25 ml) added and then washed with water. The organic solution was dried over magnesium sulphate and evaporated to give the title compound as a white powder (180 mg, 77%).

NMR 2.8 (1H, dd), 3.3 (1H, dd), 3.35 (3H, s), 4.1-4.2 (1H, m), 5.35-5.45 (1H, m), 7.1-7.3 (4H, m), 7.15 (1H, s), 8.7 (1H, d); m/z 380.9/382.9 (M+H).

### Example #149

### 2,3-Dichloro-5-(N-{1-[N-(1,1-dimethylethoxy)carbonylamino]indan-2-yl}carbamoyl)-4H-thieno[3,2-b]pyrrole

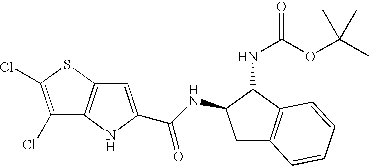

5-Carboxy-2,3-dichloro-4H-thieno[3,2-b]pyrrole (Method #9, 1.18 g, 5.0 mmol), (1R, 2R)-2-amino-1-[(1,1-dimethylethoxy)carbonylamino]indan (Method #43, 1.25 g, 5.0 mol), DIPEA (0.855 ml, 5.0 mmol), and HOBT (675 mg, 5.0 mmol) was stirred in dichloromethane (50 ml) for one minute. EDAC (1.2 g, 6.25 mmol) was added and the mixture stirred at room temperature for 20 hours. The reaction mixture was diluted with dichloromethane (50 ml), filtered and dried to give the title compound as a pale green powder (1.95 g, 85%).

NMR 1.4 (9H, s), 2.8 (1H, dd), 3,2 (1H, dd), 4.5-4.7 (1H, m), 5.1-5.2 (1H, m), 7.05-7.3 (5H, m), 7.4 (1H, d), 8.6 (1H, d), 12.4 (1H, s).

### Example #150

### 5-[N-(1-Aminoindan-2-yl)carbamoyl]-2,3-dichloro-4H-thieno[3,2-b]pyrrole

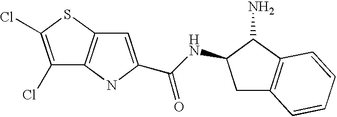

2,3-Dichloro-5-(N-{1-[(1,1-dimethylethoxy)carbonylamino]indan-2-yl}carbamoyl)-4H-thieno[3,2-b]pyrrole (Example #149, 1.0 g, 2.15 mmol) was dissolved in dichloromethane (20 ml). Trifluoroacetic acid (2 ml) was added and the mixture stirred at room temperature for 24 hours. The reaction was filtered and the isolated solid washed with dichloromethane to give the trifluoroacetic acid salt of the title compound as a pale green powder (800 mg, 78%).

NMR 3.05 (1H, dd), 3.4 (1H, dd), 4.6-4.85 (2H, m), 7.2 (1H, d), 7.3-7.45 (3H, m), 7.55 (1H, d), 8.6 (3H, broad s), 8.8 (1H, d), 12.5 (1H, s)

### Example #151

### 5-[N-(1-Acetamidoindan-2-yl)carbamoyl]-2,3-dichloro-4H-thieno[3,2-b]pyrrole

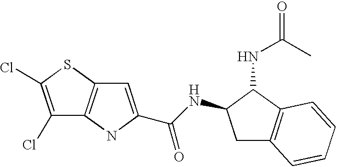

Triethylamine (101 mg, 11.0 mmol) was added to a suspension 5-[N-(1-aminoindan-2-yl)carbamoyl]-2,3-dichloro-4H-thieno[3,2-b]pyrrole trifluoroacetic acid salt (Example #150, 240 mg, 0.5 mmol) in dichloromethane (4 ml), followed by acetyl chloride (47 mg, 0.6 mmol) dissolved in dichloromethane (1 ml) and the reaction stirred at room temperature for 6 hours during which a white solid precipitated. The reaction was filtered and the crude material purified by silica chromatography with hexane:ethyl acetate to give the title compound as a white solid (50 mg, 25%). NMR 1.87 (3H, s), 2.82 (1H, dd), 3.22 (1H, dd), 4.45-4.62 (1H, m), 5.38-5.5 (1H, m), 7.02-7.27 (4H, m), 7.1 (1H, s), 8.35 (1H, d), 8.59 (1H, d), 12.36 (1H, broad s); m/z 406.13/408.8 (M−H).

### Example #152

### 2,3-Dichloro-5-{N-[1-(methanesulphonamido)indan-2-yl]carbamoyl}-4H-thieno[3,2-b]pyrrole

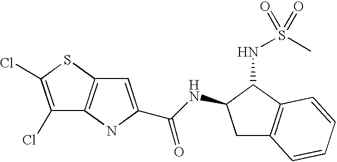

5-Carboxy-2,3-dichloro-4H-thieno[3,2-b]pyrrole (Method#9, 236 mg, 1.0 mmol), (1R,2R)-2-amino-1-methanesulphonamidoindan (Method #42, 226 mg, 1.0 mol), DIPEA (0.174 ml, 1.0 mmol), and HOBT (135 mg, 1.0 mmol) was stirred in dichloromethane (10 ml) for one minute. EDAC (240 mg, 1.25 mmol) was added and the mixture stirred at room temperature for 20 hours. The mixture was diluted with ethyl acetate, washed with water (2×25 ml), dried over magnesium sulphate and evaporated to give the title compound as a foam (400 mg, 90%). NMR 2.84 (1H, dd), 2.99 (3H, s), 3.22 (1H, dd), 4.44-4.64 (1H, m), 4.89-5.0 (1H, m), 7.14 (1H, s), 7.16-7.36 (4H, m), 7.84 (1H, d), 8.64 (1H, d), 12.43 (1H, broad s); m/z 442.2/444.0 (M−H).

### Example #153

### 2,3-Dichloro-5-{N-[1-(methylamino)indan-2-yl]carbamoyl}-4H-thieno[3,2-b]pyrrole

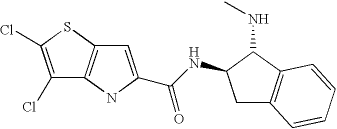

2,3-Dichloro-5-[N-(1-{N-[(1,1-dimethylethoxy)carbonyl]-N-methylamino} indan-2-yl)carbamoyl]-4H-thieno[3,2-b]pyrrole (Method #44, 900 mg, 1.87 mmol) in dichloromethane (20 ml) treated with trifluoroacetic acid (2 ml) at room temperature for 1 hour. Evaporation followed by co-evaporation with chloroform and drying gave the trifluoroacetic acid salt of the title compound as a pale brown foam (850 mg, 92%). NMR 2.75 (3H, s), 3.02 (1H, dd), 3.5 (1H, dd), 4.7-4.95 (2H, m), 7.15 (1H, s), 7.28-7.48 (3H, m), 7.6 (1H, d), 8.68 (1H, d), 9.1 (2H, broad s); m/z 380.4/382.4 (M+H).

### Example #154

### 2,3-Dichloro-5-{N-[1-(N-methylacetamido)indan-2-yl]carbamoyl}-4H-thieno[3,2-b]pyrrole

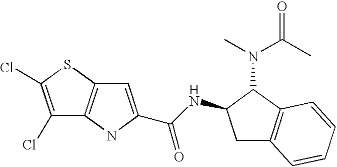

2,3-Dichloro-5-{N-[1-(methylamino)indan-2-yl]carbamoyl}-4H-thieno[3,2-b]pyrrole trifluoroacetic acid salt (Example #153, 390 mg, 0.79 mmol) in dichloromethane (5 ml) at 5° C. was treated with triethylamine (0.33 ml, 2.37 mmol) and acetyl chloride (68 mg, 0.86 mmol). After stirring at 5° C. for 15 minutes the reaction was allowed to warm to room temperature and stirred for a further 2 hours. The mixture was diluted with ethyl acetate (25 ml) and washed with saturated sodium bicarbonate and water. Drying over magnesium sulphate followed by evaporation gave the title compound as a pale brown foam (270 mg, 80%). NMR: Indicates an approximate 1:1 ratio of rotamers of the title compound; 2.05 (1.5H, s), 2.1 (1.5H, s), 2.6 (1.5H, s), 2.8 (1.5H, s), 2.9-3.08 (1H, m), 3.12-3.3 (1H, m), 4.7-4.9 (1H, m), 5.24 (0.5H, d), 6.14 (0.5H, d), 6.94-7.35 (5H, m), 8.6 (0.5H, d), 8.68 (0.5H, d), 12.38 (0.5H, broad s), 12.46 (0.5H, broad s); m/z 421.9/423.9 (M+H)

Preparation of Starting Materials

The starting materials for the Examples above are either commercially available or are readily prepared by standard methods from known materials. For example the following reactions are illustrations but not limitations of the preparation of some of the starting materials used in the above reactions.

Method #1

3-Chloro-5-methoxycarbonyl-4H-thieno[3,2-b]pyrrole

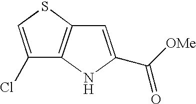

Methanolic sodium methoxide solution (28%) (5 ml, 25.9 mmol) was diluted with MeOH (5 ml) and was cooled to −25° C. under nitrogen. A solution of 4-chloro-2-thiophenecarboxaldehyde (J Heterocyclic Chem, 1976, 13, 393; 1.1 g, 7.5 mmol) and methyl azidoacetate (3.0 g, 26.1 mmol) in MeOH (20 ml) was added dropwise, maintaining the temperature at −25° C. On completion of addition the solution was allowed to warm to 5° C. over a period of approximately 16 hours. The solution was added to saturated aqueous ammonium chloride (250 ml) and the mixture was extracted using DCM. The combined organic layers were concentrated at 0° C. The residue was taken up in xylene (30 ml) and this solution was added dropwise to xylene (120 ml) under reflux. The solution was heated under reflux for 30 minutes before being cooled and concentrated. The title compound was purified by a mixture of crystallisation (EtOAc/isohexane) and chromatography on a Bond Elut column eluting with a graduated solvent of 5-50% EtOAc in isohexane (640 mg, 40%). NMR (CDCl₃) 9.1 (1H, br), 7.1 (2H, s), 3.9 (3H, s); m/z 214.3.

Methods #2-#4

The following compounds were made by the process of Method #1 using the appropriate starting materials

| Meth | Compound | NMR (CDCl₃) | M/z |
|---|---|---|---|
| #2 | 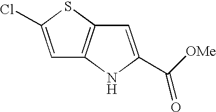 | 9.1(1H, br), 7.0(1H, s), 6.9(1H, s), 3.9(3H, s) | 214.2 |
| #3¹ | 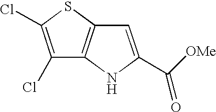 | 9.2(1H, br), 7.0(1H, s), 3.9(3H, s) | 248.2 |
| #4² | 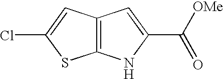 | 9.4-9.2(1H, br), 7.0(1H, s), 6.9(1H, s), 3.9(3H, s) | 214 |

¹Aldehyde: DE 2814798
²Aldehyde: Aldehyde ref. Gronowitz et al. Tetrahedron Vol. 32 1976 p. 1403

Method #5

N-Benzyl-2-(tert-butoxycarbonylamino)acetamide

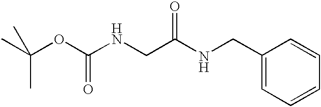

N-(tert-Butoxycarbonyl)glycine (875 mg, 5 mmol) was dissolved in DMF (7 ml) containing DIPEA (3.5 ml, 20 mmol) and benzylamine (536 mg, 5 mmol). The mixture was allowed to stand for one minute before addition of O-(7-Azabenzotriazol-1-yl)-N,N,N′,N′-tetramethyluronium hexafluorophosphate (HATU) (2.09 g, 5.5 mmol). The solution was allowed to stand for approximately 18 hours before being partitioned between ethyl acetate (50 ml) and water (50 ml). The layers were separated and the organic phase dried using magnesium sulphate, filtered, concentrated and purified using bond-elute silica column chromatography (eluent: dichloromethane-dichloromethane/methanol 5% gradient) to afford the title compound as an oil (1.32 g, quantitative).

NMR: (CDCl₃): 7.2 (5H, m), 6.3 (1H, br), 5.0 (1H, br), 4.4 (2H, d), 3.8 (2H, d), 1.4 (9H, s); m/z 265.4

Method #6

2-Amino-N-benzylacetamide

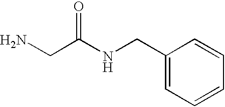

To a solution of N-benzyl-2-(tert-butoxycarbonylamino)acetamide (Method #5, 1.18 g, 4.47 mmol) in dichloromethane (6 ml) at 0° C. was added dropwise, trifluoroacetic acid (2.4 ml) and the resulting solution allowed to stir warming to room temperature overnight. The reaction mixture was neutralised by addition of saturated sodium bicarbonate solution and extracted with dichloromethane. The combined organic phases were dried over magnesium sulphate, filtered, concentrated and purified by bond-elute SCX column chromatography (eluent: methanol/dichloromethane (1:1) then methanol/dichloromethane (1:1)/ammonia 5%) to afford the title compound as an oil (215 mg, 29%).

NMR: (CDCl₃): 7.2 (6H, m), 4.4 (1.4H, d), 4.3 (0.6H, d), 3.4 (2H, br); m/z 165.17

Method #7

5-Carboxy-3-chloro-4H-thieno[3,2-b]pyrrole

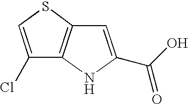

3-Chloro-5-methoxycarbonyl-4H-thieno[3,2-b]pyrrole(Method #1; 0.61 g, 2.83 mmol) was taken up in MeOH (10 ml) and was heated under reflux. Aqueous lithium hydroxide (2.0 M, 3.0 ml, 6.0 mmol) was added portionwise over 45 minutes. The mixture was heated under reflux for 30 minutes before being cooled and concentrated. Water (20 ml) was added and the solution was neutralised using aqueous hydrochloric acid (2.0 M, 3.0 ml). The solution was extracted using EtOAc, and the combined organic layers were concentrated to afford the title compound as a yellow solid (0.57 g, 100%). NMR: 12.4 (1H, br), 7.4 (1H, s), 7.0 (1H, s); m/z 200.3.

Methods #8-#10

The following compounds were made by the process of Method #7 using the appropriate starting materials.

| Meth | Compound | NMR | M/z | SM |
|---|---|---|---|---|
| #8 | 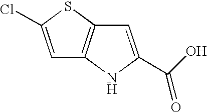 | 11.9(1H, br), 7.0(1H, s), 6.9(1H, s) | 200.1 | Method #2 |
| #9 | 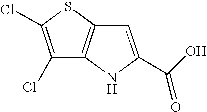 | 7.0(1H, s) | 234.2 | Method #3 |
| #10 | 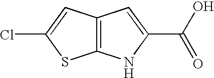 | NMR 12.6-12.7(1H, b), 12.0-12.1(1H, b), 7.15(1H, s), 6.9(1H, s) | 183 | Method #4 |

Method #11

2,3-Dichloro-5-[N-(ethoxycarbonylmethyl)carbamoyl]-4H-thieno[3,2-b]pyrrole

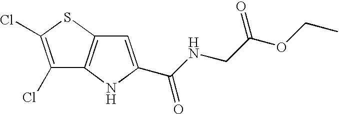

5-Carboxy-2,3-dichloro-4H-thieno[3,2-b]pyrrole (Method #9, 4.0 g 16.95 mmol) was added to a solution of glycine ethyl ester hydrochloride (2.60 g, 18.64 mmol) and DIPEA in dichloromethane (200 ml) followed by HOBT (20.29 g, 16.95 mmol). The solution was stirred under nitrogen for 15 minutes before the addition of EDAC (3.89 g, 22.03 mmol). The mixture was stirred at ambient temperature for approximately 16 hours. The resultant white precipitate was isolated by filtration, washed with water and ether and dried. (4.79 g, 88%).

NMR: 12.45 (1H, br), 8.75 (1H, t), 7.1 (1H, s), 4.1 (2H, q), 4.0 (2H, d), 1.2 (3H, t); m/z 321.2

Method #12

5-(N-Carboxymethylcarbamoyl)-2,3-dichloro-4H-thieno[3,2-b]pyrrole

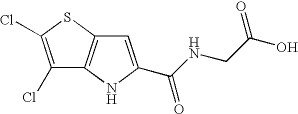

2N Sodium hydroxide solution (14.3 ml, 28.7 mmol) was added to a suspension of 2,3-dichloro-5-[N-(ethoxycarbonylmethyl)carbamoyl]-4H-thieno[3,2-b]pyrrole (Method #11, 4.60 g, 14.33 mmol) in tetrahydrofuran (THF) (100 ml). The resultant solution was stirred at room temperature for one hour. The reaction mixture was concentrated ‘in vacuo’, the residue diluted with water (250 ml), the solution adjusted to pH=2 by addition of 2N Hydrochloric acid, and then extracted with ethyl acetate (3×150 ml). The organic extracts were combined, dried over sodium sulphate then filtered and concentrated to give a white powder (3.34 g, 80%); NMR: 12.6 (1H, br), 12.4 (1H, br), 8.6 (1H, t), 7.1 (1H, s), 3.9 (2H, d); m/z 291.17

Method #13

N-(2-Cyanoethyl)-N-phenyl-2-(tert-butoxycarbonylamino)acetamide

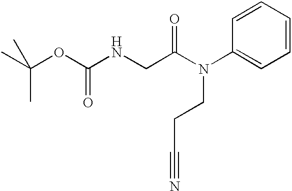

By a similar procedure to Method #5 the title compound was prepared using anilinopropionitrile to give a clear oil (598 mg, 39%); NMR: (CDCl₃): 7.4 (3H, m), 7.2 (2H, in), 5.2 (1H, br), 3.9 (2H, t), 3.6 (2H, d), 2.6 (2H, t), 1.3 (9H, s); m/z 304.51

Method #14

2-Amino-N-(2-cyanoethyl)-N-phenylacetamide

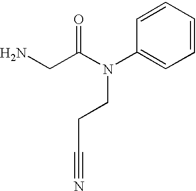

By a similar procedure to Method #6 the title compound was prepared using N-(2-cyanoethyl)-N-phenyl-2-(tert-butoxycarbonylamino)acetamide (Method #13) to give a clear oil (222 mg, 60%); NMR: (CDCl₃): 7.4 (3H, m), 7.2 (1H, d), 7.1 (1H, d), 3.9 (2H, t), 3.1 (1.33H, s), 3.0 (0.67H, s), 2.65 (1.33H, t), (0.67H, t); m/z 204.31

Method #15

N-[(2-Amino-2-(2-thiazolyl)]ethyl-N-methylmethanesulphonamide

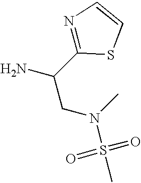

2-Bromothiazole (6.9 g, 42.0 mmol) was dissolved in dry diethyl ether (15 ml) and was added dropwise to butyl lithium (1.6 M, 29.7 ml, 47.5 mmol) in diethyl ether (40 ml) at −70° C. The mixture was stirred at −70° C. for 30 minutes before the addition of a cold solution of ethyl 2-(N-methylmethanesulphonamido)acetate (Ger Offen, 1976, 27 pp; 7.6 g, 39.0 mmol) in dry THF (70 ml). The solution was stirred for a further 30 minutes at −70° C. before being allowed to warm to ambient temperature. Aqueous ammonium chloride (10%, 200 ml) was added and the solution was extracted using diethyl ether. The aqueous layer was acidified and re-extracted using diethyl ether. The combined organic layers were dried, filtered and concentrated under reduced pressure. The residue (5.30 g, 22.6 mmol) was dissolved in ethanol (100 ml) containing pyridine (18 ml) and hydroxylamine hydrochloride (1.88 g, 27.1 mmol). The mixture was heated under reflux in an inert atmosphere for 2.5 hours before being cooled and concentrated under reduced pressure. The residue was suspended in water and cooled to 0° C. The mixture was acidified to pH 4 using aqueous hydrochloric acid (2.0 M). A solid precipitated out of solution and was filtered off and washed with water. The product was dried under reduced pressure in the presence of phosphorous pentoxide. The dry solid (4.34 g, 17.4 mmol) was dissolved in acetic acid and 5% rhodium on carbon (40% w/w, 1.7 g) was added. The solution was agitated under an atmosphere of hydrogen at 5 bar for 48 hours. The reaction vessel was flushed free of hydrogen using inert gas and the solution was filtered through celite. The filtrate was concentrated under reduced pressure. The residue was suspended in ethanol and cooled to 0° C. Aqueous hydrochloric acid (5.0 M) and ethanol (50 ml, 1:1) was added and the mixture was stirred for 30 minutes. A white precipitate was filtered off to afford the title compound (3.16 g) as the hydrochloride salt.

Method #16

Ethyl (S)-5-(□-aminophenethyl)-1,3,4-oxadiazole-2-carboxylate Trifluoroacetate

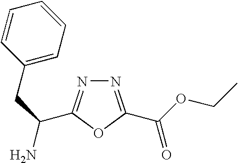

Ethyl (S)-5-[□-[(tert-butoxycarbonylamino)phenethyl]-1,3,4-oxadiazole-2-carboxylate, (Borg et al. J. Org. Chem. 1995, 60, 3112; 350 mg) was dissolved in trifluoroacetic acid (5 ml.) and allowed to stand at ambient temperature for 1 hour. The reaction mixture was concentrated and dried under vacuum to give a glassy solid (322 mg) NMR 1.75 (3H, t); 3.2-3.4 (2H, m); 4.4 (2H, q); 5.2 (1H, t); 7.1-7.4 (5H, m); 8.8-9.2 (3H, bs)

Method #17

2,3-Dichloro-5-{N-[(2-phenyl-1,3-dioxolan-2-yl)methyl]carbamoyl}-4H-thieno[3,2-b]pyrrole

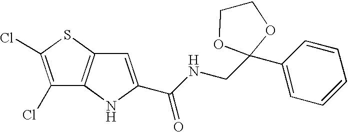

2-Phenyl-1,3-dioxolan-2-ylmethylamine hydrochloride (65 mg, 0.3 mmol) and 5-carboxy-2,3-dichloro-4H-thieno[3,2-b]pyrrole (Method #9; 71 mg, 0.3 mmol) were dissolved in DCM (10 ml) containing DIPEA (175 ml, 1.0 mmol) and HOBT (40 mg, 0.3 mmol). The mixture was stirred for one minute before the addition of EDAC (75 mg, 0.39 mmol). The solution was stirred at room temperature for approximately 18 hours. The mixture was concentrated and chromatographed on a Bond Elut column eluting with 20%-40% EtOAc in isohexane. The title compound was isolated as a white solid (100 mg). NMR: 12.4 (1H, s), 8.2 (1H, s), 7.2 (1H, s), 4.0 (2H, m), 3.7 (2H, m), 3.6 (2H, d); m/z 395.2.

Method #18

(S)-5-(□-Aminophenethyl)-3-phenyl-1,2,4-oxadiazole Trifluoroacetate

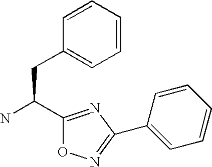

BOC-Phenylalanine (614 mg, 2.32 mmol) was dissolved in DCM (20 ml) cooled with ice/water and dicyclohexyl carbodiimide (239 mg, 1.16 mmol) added. After stirring at 0-5° C. for 1 hour the reaction mixture was filtered and concentrated in vacuo. Phenylamidoxime (104 mg, 0.77 mmol) and pyridine (10 ml) were added and the mixture heated to reflux for 2 hours. The reaction mixture was then evaporated to small volume, dissolved in ethyl acetate, washed with dilute citric acid, saturated sodium bicarbonate, water and brine, dried with magnesium sulphate and evaporated to give a crude product which was purified by chromatography on silica gel (eluted with Hexane/ethyl acetate 4:1) to (S)-5-[□-(tert-butoxycarbonylamino)phenethyl]-3-phenyl-1,2,4-oxadiazole (274 mg).

NMR 1.2-1.4 (9H, s), 3.1-3.3 (2H, m); 5.1-5.2 (1H, m); 7.2-7.3 (5H, m); 7.5-7.6 (3H, m); 7.8 (1H, d); 7.95-8.05 (2H, m); m/z 364

(S)-5-[□-(tert-Butoxycarbonylamino)phenethyl]-3-phenyl-1,2,4-oxadiazole (274 mg) was dissolved in trifluoroacetic acid (5 ml) and stirred at ambient temperature for 2 hours. After evaporation and drying under vacuum the title compound was obtained as a pale yellow solid (232 mg). NMR 3,2-3.5 (2H, m); 5.15 (1H, t); 7.1-7.3 (5H, m); 7.4-7.6 (3H, m); 7.9 (2H, d); 9.0 (3H, s); m/z 266

Method #19

3-(1H-Tetrazol-5-yl)propylamine

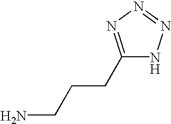

4-{[(Phenylacetyl)oxy]amino}butanamide (J. Biol. Chem., 1971, 246, 6683) (16 g) was dissolved in dry pyridine (150 ml) and the solution was cooled to −10° C. A solution of POCl₃ (8.1 ml) in DCM (16.5 ml) was added dropwise over 30 minutes. The mixture was stirred at ambient temperature for 1 hour before being diluted with water until the pH reached 5. The solution was extracted using EtOAc. The combined organic layers were washed with dilute aqueous hydrochloric acid and water before being dried, filtered and concentrated. The resulting solid (9.2 g, 42.8 mmol) was dissolved in dry DMF (30 ml) and the solution was heated on a steam bath for 2 hours. Ammonium chloride (2.22 g, 41.5 mmol) was added to the hot solution along with sodium azide (2.68 g, 56.3 mmol). The mixture was cooled and left to stir for over 48 hours. The solution was filtered and basified to pH 8 using aqueous potassium bicarbonate. The aqueous layer was washed using EtOAc before being acidified using dilute aqueous hydrochloric acid. A white solid was filtered off. This solid (3.0 g, 11.5 mmol) was dissolved in acetic acid (50 ml) and water 5 ml. Palladium on charcoal (5%, 400 mg) was added and the solution was shaken under an atmosphere of hydrogen for 6.5 hours. The suspension was filtered and washed with acetic acid. The filtrate was concentrated and dried. The residue was treated with iso-propanol and the title compound was filtered off as a white solid (1.37 g, 94%).

Method #20

2-Amino-N-(2-hydroxy-3-phenoxypropyl)acetamide

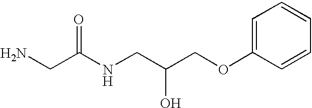

N-Benzyloxycarbonylglycine (2.09 g) was dissolved in toluene (40 ml) and DMF (5 drops). Oxalyl chloride (1.3 ml) was added and the mixture was stirred at ambient temperature for 2 hours. The solution was diluted with diethyl ether (25 ml) and was added dropwise to a solution of 1-amino-3-phenoxy-2-propanol (1.67 g) in diethyl ether (25 ml). Sodium hydroxide (0.4 g) was dissolved in water (1.5 ml) and was added to the mixture. The solution was stirred for greater than 48 hours before being filtered. The sticky solid isolated was added to saturated aqueous sodium bicarbonate (100 ml) and was stirred for 15 minutes before being filtered. The solid was recrystallized from an EtOAc/petrol mixture. This resulting compound (650 mg) was dissolved in MeOH (10 ml) and palladium on charcoal (5%, 50 mg) was added along with acetic acid (1 ml). The solution was stirred under an atmosphere of hydrogen for 4 hours before being filtered. The filtrate was concentrated to afford an oily residue. The residue was recrystallized from EtOH/diethyl ether mixture to afford the title compound (200 mg).

Method #21

2-Amino-N-(3-methylisothiazol-5-yl)acetamide

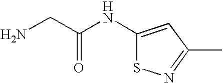

N-t-Butoxycarboylglycine (1.92 g, 1.1 mmol) was dissolved in dry EtOAc (20 ml) and the solution was cooled to −25° C. N-Methyl morpholine (1.1 g, 1.1 mmol) was added and the mixture was stirred for 2 minutes before the addition of ethyl chloroformate (1.08 g, 1.0 mmol). A white solid precipitated from solution. A suspension of 5-amino-3-methylisothiazole hydrochloride (1.5 g, 1.0 mmol) was added in triethylamine (1.01 g, 1.0 mmol) and dry EtOAc (10 ml). The mixture was stirred at ambient temperature for 17 hours before being filtered. The filtrate was concentrated and the residue was purified by flash column chromatography, using diethyl ether as eluent. The residue (271 mg, 0.1 mmol) was dissolved in EtOAc (3 ml). Ethanolic HCl (1 ml) was added and the mixture was stirred at ambient temperature for 2.5 hours. The title compound precipitated as a white solid and was isolated by filtration (220 mg, 92%).

Method #22

2-(Pyridazin-3-yloxy)ethylamine

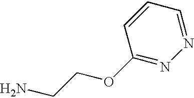

Ethanolamine (6.0 ml) was suspended in dry xylene (100 ml) and the mixture was stirred at ambient temperature with sodium hydride (3 g). After 20 minutes the solution was cooled to 0° C. 3,6-Dichloropyridazine (15.0 g) was added. The solution was warmed to ambient temperature and was stirred for 15 hours before being extracted with chloroform. The chloroform was concentrated and the residue was taken up in EtOAc before being filtered. Ethanolic HCl was added and a white solid was filtered off. This compound (550 mg) was dissolved in MeOH (130 ml) and palladium on charcoal (5%, 200 mg) was added. The suspension was stirred under an atmosphere of hydrogen for 6 hours before being filtered. The filtrate was concentrated and the residue was triturated using diethyl ether. The title compound was isolated as a white solid (340 mg).

Method #23

1-(2-Aminoethyl)imidazolidine-2,4-dione

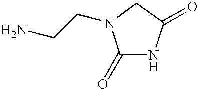

Ethylene diamine (30 ml) was dissolved in DCM (130 ml) with di-t-butyl dicarbonate (13.5 g). The mixture was stirred at ambient temperature for 30 minutes before the solvent was decanted off. The residue was washed with water and dried. The product (6.1 g) was dissolved in ethanol (15 ml). Chloroacetic acid (5.77 g) solution in aqueous sodium hydroxide (1 M, 61 ml) was added dropwise, followed by more aqueous sodium hydroxide (1 M, 31 ml). The mixture was stirred for 17 hours before being washed with diethyl ether. Benzyl chloroformate (5 g) was added to the aqueous phase and the mixture was stirred at ambient temperature for 4 hours. The solution was washed with diethyl ether and the aqueous layer was acidified using aqueous citric acid (30%). The solution was extracted using EtOAc. The combined organic layers were washed with brine, then water before being dried, filtered and concentrated. The residue was triturated using diethyl ether/petrol mixture and the required intermediate was isolated as a white solid. Following repeated procedure on a larger scale, this solid (28 g) was dissolved in ethanol (250 ml) containing palladium on charcoal (10%, 4.6 g). The suspension was stirred under an atmosphere of hydrogen until the reaction was complete. The mixture was filtered and the filtrate was concentrated. The residue (3 g, was treated with potassium cyanate (1.3 g) in water (30 ml) and the mixture was heated under reflux for 2 hours. An excess of concentrated hydrochloric acid was added and the mixture was heated for a short time before being concentrated. The residue was taken up in water (50 ml) and was loaded onto basic Amberlite® IRA resin. The resin was washed with water until the eluent was found to be neutral. Dilute aqueous hydrochloric acid was used to elute the crude product from the resin. The acidic fractions were concentrated and the residue was triturated with ethanol. The title compound was isolated as orange crystals (950 mg).

Method #24

N-(4-Aminobutylyl)methanesulphonaamide

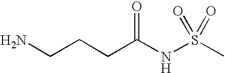

4-Phthalimidobutyryl chloride (J Am. Chem. Soc., 1981, 103, 6750) (1.27 g) was heated with methane sulphonamide (480 mg) at 100° C. for 15 min under argon. The mixture was cooled and left to stand for 17 hours. The residue was triturated with ethanol to afford the required intermediate. The solid (4 g) was treated with potassium hydroxide (75 g) in water (100 nil) at ambient temperature for 2 hours. Concentrated hydrochloric acid was added until the pH of the solution reached 9. The solution was extracted using EtOAc. The combined organic layers were concentrated and the residue was triturated using diethyl ether/petrol mixture and filtered. The residue (5.7 g) was taken up in water (150 ml) and dilute hydrochloric acid (1 M) was added until the pH reached 1. The mixture was heated on a steam bath for 1 hour before being cooled and extracted using EtOAc. The aqueous phase was concentrated and the residue was triturated using EtOAc/ethanol mixture to afford the title compound (1.5 g).

Method #25

2-[3-(2-Aminoethoxy)phenyl]acetamide

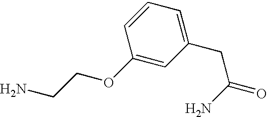

3-Hydroxyphenylacetic acid (30.4 ml) was dissolved in MeOH (160 ml). Concentrated sulphuric acid (1.6 ml) was added and the mixture was heated under reflux for 6 hours. The mixture was concentrated to low bulk before the addition of toluene (120 ml). The mixture was washed with water, saturated aqueous sodium bicarbonate and brine. The combined organic layers were reduced in volume by one half before being stirred with ammonia solution (180 ml) for 16 hours. The mixture was concentrated and filtered. The solid was washed with water and was dried. The residue (24 g) was dissolved in MeOH (400 ml) with 1,2-dibromoethane (20 ml) and sodium hydroxide (6 g). The solution was heated under reflux for 36 hours before being concentrated. The residue was partitioned between water and EtOAc. The organic phase was separated and concentrated. This residue was purified by medium pressure liquid chromatography, using 5% MeOH in DCM as eluent. The purified intermediate (7.5 g) was dissolved in ethanol (300 ml) with ammonia solution (500 ml). The reaction vessel was sealed and the mixture was stirred at ambient temperature for 6 hours before being allowed to stand for 2.5 days. The solution was concentrated and the residue was purified using medium pressure liquid chromatography, using 25% MeOH in DCM as eluent, to afford the title compound (4.1 g).

Method #26

(trans)-4-Amino-5-hydroxy-1,3,4,5-tetrahydro-2H-1-benzazepin-2-one

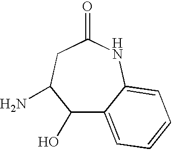

Anhydrous toluene (33 ml) and ethanol (2.2 ml) were heated with potassium (0.3 g) until all the metal has dissolved. 3,4-Dihydro-1H-1-benzazepine-2,5-dione (J. Org. Chem., 1972, 37, 208) (11.6 g) was added and the mixture was heated under reflux for 2 minutes before being cooled to ambient temperature. Butyl nitrite (1.7 ml) was added and the mixture was stirred for 4 hours before being left to stand for 3 days. The solvent was decanted and the residue was passed down a Fluorisil® column, using 5% MeOH in chloroform as eluent. The product (1.0 g) was dissolved in water (40 ml) and MeOH (14 ml) containing palladium on charcoal (5%, 500 mg). The mixture was stirred under an atmosphere of hydrogen for 1 hour. The mixture was filtered and the filtrate was concentrated. The residue was recrystallized using ethanol to afford the title compound.

Method #27

(2R,5S)-2-Aminomethyl-5-(1,3-benzodioxol-5-yl)-4-methylmorpholine

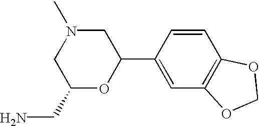

A solution of 1-(1,3-benzodioxol-5-yl)-2-(methylamino)ethanol (Synthesis, 1979, 423) (28.65 g) and oxirane-2-carboxamide (Bull. Chem. Soc. Jpn., 1989, 62, 3202) (14.0 g) in ethanol (500 ml) was heated under reflux for 1.5 hours. The solution was concentrated and the residue was triturated using diethyl ether. A white powder was filtered off. This product (3.0 g) was added portionwise to trifluoroacetic acid (30 ml) at ambient temperature. The solution was stirred for 45 minutes before being concentrated. The residue was basified using saturated aqueous sodium bicarbonate. The mixture was filtered and the filtrate was concentrated. The residual oil was purified by flash column chromatography, using a gradient of MeOH in EtOAc as eluent, to afford both cis- and trans-isomers of the required intermediate. The trans-isomer (1.6 g) was dissolved in dry diethyl ether (180 ml). The solution was heated under reflux using Soxhlet equipment with lithium aluminium hydride (2.0 g) for 16 hours. The solution was cooled and water (2 ml) was added with sodium hydroxide solution (3 M, 2 ml), followed by more water (6 ml). The metal residues were removed by filtration and the filtrate was extracted using DCM. The combined organic layers were dried, filtered and concentrated. The residue was triturated using ethanol to afford the title compound as a white solid.

Method #28

N-Methyl-N-phenyl-2-(tert-butoxycarbonylamino)acetamide

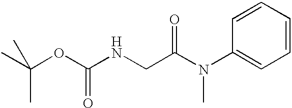

By a similar procedure to Method #5 the title compound was prepared using N-methylaniline to give a pale orange oil (771 mg, 58%); NMR: (CDCl₃): 7.4 (3H, m), 7.1 (2H, m), 5.3 (1H, br), 3.6 (2H, br), 3,2 (3H, s), 1.4 (9H, s); m/z 265.42

Method #29

N-Benzyl-N-phenyl-2-(tert-butoxycarbonylamino)acetamide

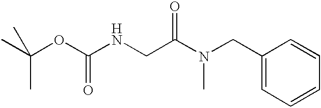

By a similar procedure to Method #5 the title compound was prepared using N-benzylmethylamine to give a yellow oil (670 mg, 48%); NMR: (CDCl₃): 7.2 (5H, m), 5.5 (1H, br), 4.6 (1.33H, s), 4.4 (0.67H, s), 3.9 (2H, br), 2.9 (1H, s), 2.8 (2H, s), 1.4 (9H, s); m/z 279.50

Method #30

2-Amino-N-methyl-N-phenylacetamide

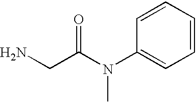

By a similar procedure to Method #6 using N-methyl-N-phenyl-2-(tert-butoxycarbonylamino)acetamide (Method #28) the title compound was prepared to give the title compound as a clear oil (231 mg, 56%); NMR: (CDCl₃): 7.3 (3H, m), 7.1 (2H, d), 3,2 (3H, s), (2H, br); m/z 165.17

Method #31

2-Amino-N-methyl-N-benzylacetamide

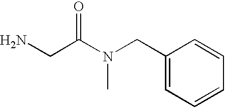

By a similar procedure to Method #6 using N-benzyl-N-phenyl-2-(tert-butoxycarbonylamino)acetamide (Method #29) the title compound was prepared to give the title compound as a clear oil (256 mg, 73%); NMR: (CDCl₃): 7.3 (5H, m), 4.6 (1.33H, s), 4.4 (0.67H, s), 3.4 (2H, br), 2.95 (1.33H, s), 2.8 (2.34H, s), (1.33H, s); m/z 179.23

Method #32

Methyl (S)-5-(□-aminophenethyl)oxazole-4-carboxylate Trifluoroacetate

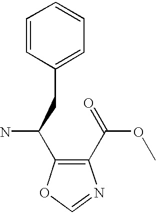

Methyl (S)-5-[□-(tert-butoxycarbonylamino)phenethyl]-oxazole-4-carboxylate, (Tert. Lett., 1982, 23, 235; 417 mg) was dissolved in trifluoroacetic acid (3 ml) and stood at ambient temperature for 1 hour and concentrated to give an oil. Trituration with diethyl ether gave the title compound as a white solid. (281 mg); NMR 3.1-3.25 (1H, m); 3.3-3.4 (1H, m); 3.7 (3H, s); 5.2-5.3 (1H, m); 7.05 (2H, d); 7.2-7.3 (3H, m); 8.7 (1H, s); 8.75-8.85 (3H, bs)

Method #33

3-Aminomethyl-4-phenylisoxazole

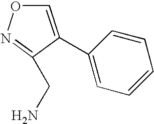

4-Phenylisoxazole-3-carboxylic acid ethyl ester (JOC 50 13 2372 1983; 404 mg, 1.86 mmol) dissolved in THF 10 ml under nitrogen was treated with 2M lithium borohydride in THF (1.86 ml, 3.72 mmol). The resultant mixture was stirred at 0C for 5 hours, then allowed to warm to room temperature overnight. 1M acetic acid was added dropwise till effervesence ceased, a further 20 ml of water was added and the resultant solution extracted with ethyl acetate (3×20 ml), the organic extracts were washed with saturated sodium bicarbonate solution (10 ml) and saturated brine (10 ml), dried with anhydrous magnesium sulphate and evaporated to dryness under reduced pressure to give 3-hydroxymethyl-4-phenylisoxazole (295 mg); NMR (CDCl₃): 4.9 (2H, d), 7.3-7.5 (5H, m), 8.5 (1H, s) m/z 176 (M+H)

3-Hydroxymethyl-4-phenylisoxazole (295 mg, 1.68 mmol) was dissolved in THF 5 ml under nitrogen together with phthalimide (292 mg, 1.68 mmol) and triphenylphosphine (440 mg, 1.68 mmol), the resultant stirred solution was cooled to 0C and treated with DIAD (330 ul, 1.68 mmol) dropwise over 30 mins, then allowed to warm to room temperature overnight. After evaporation to dryness the mixture was purified by chromatography on a 20 g Bond Elute silica column eluting with 1:1 DCM:hexane to give 4-phenyl-3-phthalimidomethylisoxazole (316 mg): m/z 305 (M+H). This was dissolved in methanol 3 ml, treated with hydrazine hydrate (114 ul, 2.4 mmol) and refluxed for 30 mins. The resultant suspension was filtered, the solid washed with ethanol (2×5 ml) and the filtrate then evaporated to dryness, this residue was taken up in 2N HCl and any insoluble material again removed by filtration. The filtrate was basified with saturated sodium bicarbonate and extracted with ethyl acetate (3×10 ml), the combined organic extracts were washed with water (5 ml) and saturated brine (5 ml), then dried over magnesium sulphate and evaporated under reduced pressure to give 3-aminomethyl-4-phenylisoxazole (130 mg); NMR (CDCl3): 4.1 (2H, s), 7.3-7.5 (5H, m), 8.4 (1H, s); m/z 175 (M+H)

Method 434

2-[2-(2-morpholinoethoxy)phenyl]ethylamine

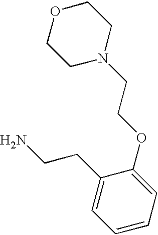

2-(2-Hydroxyphenyl)ethylamine (1.73 g, 10 mmol) dissolved in DCM 60 ml was treated with a solution of sodium bicarbonate (1.68 g, 20 mmol) in water, the emulsion was stirred vigorously in an ice bath and benzyl chloroformate (1.785 g, 10.5 mmol) was added dropwise over 10 mins and the mixture was stirred overnight at room temperature. The DCM phase was removed and the aqueous phase extracted with DCM (2×20 ml). The combined organics were then dried over magnesium sulphate and evaporated under reduced pressure, the product was purified by chromatography on 20 g silica bond elute, eluting with DCM to give N-(benzyloxycarbonyl)-2-(2-hydroxyphenyl)ethylamine (1.4 g); NMR (CDCl3): 2.8 (2H, q), 3.4 (2H, q), 5.1 (3H, m), 6.4 (1H, s), 6.8 (2H, m), 7.1 (2H, m), 7.3-7.4 (5H, m)

N-(Benzyloxycarbonyl)-2-(2-hydroxyphenyl)ethylamine (542 mg, 2 mmol) dissolved in DMF 5 ml was treated with potassium carbonate (325 mesh, 350 mg, 2.5 mmol) and 2-chloroethyl p-toluenesulphonate (484 ul, 2.6 mmol) and the mixture was stirred at 60C overnight. After cooling, water 10 ml was added and the mixture extracted with diethyl ether (3×20 ml) dried over magnesium sulphate and evaporated under reduced pressure, the product was purified by chromatography on 20 g silica bond elute, eluting with DCM to give N-(benzyloxycarbonyl)-2-[2-(2-chloroethoxy)phenyl]ethylamine(450 mg); NMR (CDCl3): 2.9 (2H, q), 3.5 (2H, m), 3.8 (2H, m), 4.2 (2H, m), 4.9 (1H, b), 5.1 (2H, s), 6.8 (1H, d), 6.9 (1H, t), 7.1 (2H, m), 7.3-7.4 (5H, m) HPLC Hichrome C18 column Acetonitrile/water/0.1%TFA 5-95% over 7.5 min Rt 4.98 min

N-(Benzyloxycarbonyl)-2-[2-(2-chloroethoxy)phenyl]ethylaamine (110 mg, 0.33 mmol) dissolved in NMP 3 ml was heated with morpholine (174 ul, 2 mmol) at 80C overnight. Water (5 ml) was added and the mixture extracted with ethyl acetate (30 ml), the organic phase was washed with water (2×5 ml) dried over magnesium sulphate and evaporated under reduced pressure to give N-(benzyloxycarbonyl)-2-[2-(2-morpholinoethoxy)phenyl]ethylamine (143 mg); NMR (CDCl3): 2.5 (4H, q), 2.8 (4H, m), 3.4 (2H, q), 3.7 (4H, q), 4.1 (2H, t), 5.0 (2H, s), 5.3 (1H, b), 6.8 (2H, m), 7.1 (1H, d), 7.2 (1H, t), 7.3-7.4 (5H, m) HPLC Hichrome C18 column Acetonitrile/water/0.1%TFA 5-95% over 7.5 min Rt 3.73 min m/z 385 (M+H)

N-(Benzyloxycarbonyl)-2-[2-(2-morpholinoethoxy)phenyl]ethylamine (143 mg) dissolved in methanol was treated with 10% palladium on carbon and hydrogenated for 2 hours. The solid was removed by filtration, washed with methanol (2×2 ml), the filtrate evaporated under reduced pressure and azeotroped twice with toluene to give the title compound (83 mg) m/z 251 (M+H), which was used directly in the next step.

Method #35

Methyl 2-[2-(2-aminoethyl)phenoxy]acetate

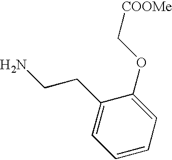

N-(Benzyloxycarbonyl)-2-(2-hydroxyphenyl)ethylamine (see Method #34: 718 mg, 2.65 mmol) dissolved in DMF 6 ml was treated with potassium carbonate (−325 mesh, 397 mg, 4.0 mmol) methyl bromoacetate (362 ul, 3.7 mmol) and the mixture was stirred overnight at room temperature. After cooling, water 10 ml was added and the mixture extracted with diethyl ether (3×20 ml) dried over magnesium sulphate and evaporated under reduced pressure to give methyl 2-{2-[2-(benzyloxycarbonyl(amino)ethyl)]phenoxy}acetate (847 mg); NMR (CDCl3): 2.8 (2H, q), 3.5 (2H, m), 3.8 (3H, s), 4.6 (2H, s), 5.1 (3H, m), 6.7 (1H, d), 6.9 (1H, t), 7.1-7.4 (7H, m)

Methyl 2-{2-[2-(benzyloxycarbonylamino)ethyl]phenoxy} acetate (343 mg, 1 mmol) dissolved in methanol together with p-toluenesulphonic acid (190 mg, 1 mmol) was treated with 10% palladium on carbon and hydrogenated for 2 hours. The solid was removed by filtration, washed with methanol (2×2 ml), the filtrate evaporated under reduced pressure and azeotroped twice with toluene to give methyl 2-[2-(2-aminoethyl)phenoxy]acetate(403 mg), m/z 210 (M+H) which was used without further purification.

Method #36

2-Aminoindan-1-ol

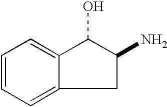

Isoamyl nitrite (15 ml, 108 mmol) was added to a solution of indan-1,2-dione (12 g, 90 mmol) in methanol (380 ml) at 45° C. followed by concentrated HCl (12 ml) dropwise over 5 minutes. The reaction mixture was stirred for 3 hours at room temperature. Excess isoamyl nitrite (1 ml) and concentrated HCl (1 ml) was added and the suspension stirred for a further 15 minutes. On cooling to room temperature a white precipitate formed. The precipitate was filtered off and washed with cold methanol (40 ml) followed by diethyl ether (40 ml) to afford indan-1,2-dione-2-oxime as a white solid (6.2 g, 43%).

NMR: 3.8 (2H, s), 7.4 (1H, t), 7.6 (1H, d), 7.7 (2H, t); m/z 162 (M+H)

A solution of indan-1,2-dione-2-oxime (6.2 g, 39 mmol) in ethanol (470 ml) and 4MHCl/Dioxane (36 ml) was hydrogenated at room temperature and 40 psi. The reaction mixture was filtered through celite, washed with ethanol (30 ml) and concentrated under reduced pressure to give 10 g of an off-white solid which was recrystallised from ethanol to give the title compound as a white solid (5 g, 86%). NMR: 2.8 (1H, dd), 3,2 (1H, dd), 3.7 (1H, q), 5.1 (1H, t), 6.0 (1H, d), 7.1-7.3 (4H, m), 8.6 (2H, s)

Method #37

2-Amino-N-methyl-N-(1-oxo-1,2,3,4-tetrahydronaphth-2-yl)methylacetamide

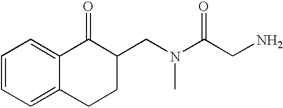

Tetralone (1 eq, 100 mmol, 15 g) and methylamine hydrochloride (2.5 eq, 250 mmol, 15 g) and paraformaldehyde (1 eq, 100 mmol, 3 g) in ethanol (100 ml) were heated under reflux for 16 hours. The reaction mixture was cooled and filtered to afford 2-(methylaminomethyl)-3,4,dihydro-1-(2H)-naphthalenone as a solid (14 g).

2-(Methylaminomethyl)-3,4,dihydro-1-(2H)-naphthalenone (1 eq, 20 mmol, 4.5 g), HOBT (1 eq, 20 mmol, 2.7 g), N-benzyloxycarbonylglycine (1 eq, 20 mmol, 4.18 g), triethylamine (1.25 eq, 25 mmol, 3 ml) and dicyclohexylcarbodi-imide (1 eq, 20 mmol, 4.12 g) were dissolved in dichloromethane (130 ml) and were stirred at ambient temperature for 18 hours before being cooled to 0° C. and filtered. The filtrate was concentrated and the residue was taken up in ethyl acetate before being washed with aqueous sodium bicarbonate, dried, filtered and concentrated. The residue was left to stand in a solution of hydrogen bromide in acetic acid for 30 minutes. The mixture was triturated with diethyl ether and the solvent was decanted off. The residue was treated with ammonia solution and this was extracted with chloroform. The chloroform was concentrated and the residue was recrystallised from ethanol to afford the title compound.

Method #38

2-Amino-6-fluoro-1-indanol

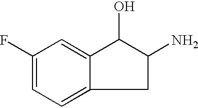

To a solution of sodium carbonate (0.8 g, 7.2 mmol) in water (13 ml) was added 4-fluorophenylalanine (1.3 g, 7.2 mmol) followed by a solution of N-ethoxycarbonylphthalamide (1.6 g, 7.2 mmol) in ethyl acetate (10 ml). The two-phase reaction mixture was stirred for 24 hours at room temperature. The organic phase was separated and discarded and the aqueous phase was acidified with concentrated HCl to pH 2. The aqueous phase was then extracted with ethyl acetate (3×20 ml) and the combined extracts dried over magnesium sulphate then concentrated under reduced pressure to give 3-(4-fluorophenyl)-2-phthalimidopropanoic acid as a white solid (2 g, 89%); NMR 3.3 (1H, dd), 3.5 (1H, dd), 5.1 (1H, dd), 6.3 (1H, brs), 7.0 (2H, t), 7.1 (2H, dd), 7.8 (4H, s).

To a solution of 3-(4-fluorophenyl)-2-phthalimidopropanoic acid (2.0 g, 6.4 mmol) in DCM (20 ml) was added thionylchloride (0.6 ml, 6.4 mmol) and 1 drop of DMF. The reaction mixture was stirred at room temperature for 1 hour. Aluminium chloride (2.6 g, 19.2 mmol) was added and the reaction mixture stirred for 3 hours at room temperature. The resulting mixture was poured into ice and concentrated HCl (20 ml) and stirred for 10 minutes. This mixture was extracted with DCM (3×20 ml) and the combined organic extracts were washed with water (2×20 ml), saturated sodium hydrogen carbonate (1×20 ml) and water (1×20 ml). The organic phase was then dried over magnesium sulphate and concentrated under reduced pressure to give 6-fluoro-2-phthalamido-indan-1-one as a white solid (1 g, 53%). NMR 3.3 (1H, dd), 3.5 (1H, dd), 5.1 (1H, dd), 6.8 (1H, t), 7.0 (1H, dd), 7.7-8.0 (5H, m); m/z 296 (M+H)

Sodium borohydride (512 mg, 14 mmol) was added to a solution 6-fluoro-2-phthalamido-indan-1-one (800 mg, 2.7 mmol) in isopropanol/water (6:1, 10.5 ml) and the solution stirred at room temperature for 24 hours. Excess acetic acid was then added to the reaction mixture and the resulting solution heated at 60° C. for 6 hours. The reaction mixture was then cooled to room temperature and concentrated under reduced pressure to give a white solid which was purified by ion-exchange chromatography using Dowex 50w×2 (water:methanol 1:1 containing 3% ammonia) to give the title compound as a white solid (141 mg, 31%); NMR 3.3 (1H, dd), 3.5 (1H, dd), 4.8 (1H, m), 4.9 (1H, d), 6.8-7.4 (3H, m); m/Z 168 (M+H)

Method #39

2-Amino-1,2,3,4-tetrahydronaphth-1-ol

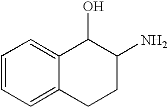

To a solution of sodium carbonate (0.8 g, 7.2 mmol) in water (13 ml) was added homophenylalanine (1.3 g, 7.2 mmol) followed by a solution of N-ethoxycarbonylphthalamide (1.6 g, 7.2 mmol) in ethyl acetate (10 ml). The two-phase reaction mixture was stirred for 24 hours at room temperature. The organic phase was separated and discarded and the aqueous phase was acidified with concentrated HCl to pH 2. The aqueous phase was then extracted with ethyl acetate (3×20 ml) and the combined extracts dried over magnesium sulphate then concentrated under reduced pressure to give 4-phenyl-2-phthalimidobutanoic acid as a white solid (1.5 g, 67%); m/z 308 (M−H).

To a solution of 4-phenyl-2-phthalimidobutanoic acid (1.5 g, 4.9 mmol) in DCM (20 ml) was added thionylchloride (0.4 ml, 4.9 mmol) and 1 drop of DMF. The reaction mixture was stirred at room temperature for 1 hour. Aluminium chloride (2.0 g, 14.7 mmol) was added and the reaction mixture stirred for 3 hours at room temperature. The resulting mixture was poured into ice and concentrated HCl (20 ml) and stirred for 10 minutes. This mixture was extracted with DCM (3×20 ml) and the combined organic extracts were washed with water (2×20 ml), saturated sodium hydrogen carbonate (1×20 ml) and water (1×20 ml). The organic phase was then dried over magnesium sulphate and concentrated under reduced pressure to give 2-phthalamido-3,4-dihydro-(2 h)-naphthalen-1-one as a white solid (880 mg, 62%). NMR 2.3 (1H, m), 2.7 (1H, m), 3.1 (1H, dt), 3.3 (1H, m), 5.2 (1H, dd), 7.3-8.0 (8H, m); m/Z 292 (M+H)

To a solution of 2-phthalamido-3,4-dihydro-(2H)-naphthalen-1-one (880 mg, 3 mmol) in isopropanol/water (6:1, 11.5 ml) was added sodium borohydride (567 mg, 15 mmol) and the solution stirred at room temperature for 24 hours. Excess acetic acid was then added to the reaction mixture and the resulting solution heated at 60° C. for 6 hours. The reaction mixture was then cooled to room temperature and concentrated under reduced pressure to give a white solid which was purified by ion-exchange chromatography using Dowex 50w×2 (water:methanol 1:1 containing 3% ammonia) to give the title compound as a white solid (150 mg, 30%). NMR 2.8 (1H, dd), 3.0 (1H, dd), 3.3 (1H, m), 3.5 (1H, m), 4.5 (1H, m), 4.8 (1H, d), 7.1-7.4 (4H, m); m/z 164 (M+H)

Method #40

(+/−) Trans-2-Amino-1-methoxyindan

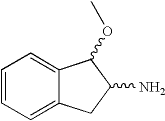

A solution of (+/−) trans-2-bromo-1-hydroxyindan (21.0 g, 0.1 mol) and potassium phthalimide (42.0 g, 0.22 mol) in dry DMF (120 ml) was heated at 100° C. for 5 hours. The reaction mixture was cooled and evaporated to an oil which was triturated with ethyl acetate and filtered. The filtrates were evaporated and purified by chromatography on silica with 2:1 iso-hexane:ethyl acetate as eluent to give (+/−)-trans-1-hydroxy-2-phthalimido indan as a pale yellow amorphous powder (17.7 g, 63%). NMR 2.8 (1H, dd), 3.15 (1Hdd), 4.8-5.0 (1H, m), 5.4 (1H, d), 5.45 (1H, d), 7.0-7.3 (4H, m), 7.75-8.95 (4H, m).

To a solution of (+/−) trans-1-hydroxy-2-phthalimidoindan (1.4 g, 5.0 mmol) in dry tetrahydrofuran (20 ml) was added 60% sodium hydride (300 mg, 7.5 mmol). The mixture was stirred at room temperature for 2 hours and then methyl iodide (0.62 ml, 10.0 mmol) added. The mixture was stirred for a further 2 hours and then 5% water in tetrahydrofuran (10 ml) and ethyl acetate (75 ml) added. The solution was washed with water, dried over magnesium sulphate and evaporated to give (+/−)-trans-1-methoxy-2-phthalimidoindan as a white solid (1.2 g, 82%). NMR 2.85 (1H, dd), 3.25 (3H, s), 3.5 (1H, dd), 4.5-4.65 (1H, m), 5.55 (1H, d), 7.05-7.3 (4H, m), 7.85 (4H, s).

A mixture of (+/−)-trans-1-methoxy-2-phthalimidoindan (850 mg, 2.9 mmol) and hydrazine hydrate (5 ml) in ethanol was stirred at room temperature for 24 hours. The mixture was evaporated and purified by ion exchange chromatography (Dowex 50W×2H⁺ form) and the title compound eluted with 50% aqueous methanol containing 3% ammonium hydroxide to give the title compound a pale yellow solid (400 mg, 85%). NMR 2.8 (1H, dd), 3.35 (3H, s), 3.38 (1H, dd),4.05-4.15 (1H, m), 4.5-4.6 (1H, m), 7.2-7.35 (3H, m), 7.45-7.55 (1H, m); m/z 164 (M+H).

Method #41

(1R,2S)-1-[(1,1-dimethylethoxy)carbonylamino]-2 hydroxyindan

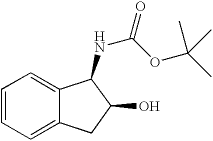

(1R,2S)-1-Amino-2-hydroxyindan (10.0 g, 67.1 mmol) was dissolved in dichloromethane (550 ml) and triethylamine (18.7 ml, 134.2 mmol). Di-tert-butyl dicarbonate (18.3 g, 83,9 mmol) in dichloromethane (50 ml) was added and the mixture stirred at room temperature for 20 hours then evaporated. Ethyl acetate (200 ml) was added, the solution washed with water, dried over magnesium sulphate and evaporated. The crude product was purified by chromatography on silica with 4:1 iso-hexane:ethyl acetate as eluent to give the title compound as a white solid (16.1 g, 96%); NMR 1.42 (9H, s), 2.78 (1H, dd), 3.0 (1H, dd), 4.3-4.42 (1H, m), 4.78-4.9 (1H, m), 4.9-5.0 (1H, m), 6.3 (1H, d), 7.0-7.25 (4H, m).

Method #42

(1R,2R)-2-Amino-1-methanesulphonamidoindan

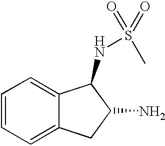

(1R,2S)-1-Amino-2-hydroxyindan (3.0 g, 20 mmol) was dissolved in dry tetrahydrofuran (40 ml) and triethylamine (8.4 ml, 60.0 mmol) at 10° C. Methane sulphonyl chloride (5.0 g, 44.0 mmol) dissolved in tetrahydrofuran (10 ml) was added at such a rate that the internal temperature remained below 15° C. Following the addition the mixture was stirred at room temperature for 20 hours and then evaporated. To the residue was added ethyl acetate (100 ml) and the mixture washed with saturated aqueous sodium bicarbonate and then water. The organic solution was dried over magnesium sulphate and evaporated to give (1R,2S)-1-methanesulphonamido-2-methylsulphonyloxyindan as a pale yellow solid (5.7 g, 93%).

NMR: 3.0-3.35 (2H, m), 3.1 (3H, s), 3.25 (3H, s), 5.05-5.2 (11H, m), 5.3-5.4 (1H, m), 7.2-7.4 (4H, m), 7.85-8.0 (1H, m).

m/z 304.2 (M−H).

(1R,2S)-1-Methanesulphonamido-2-methylsulphonyloxyindan (2.0 g, 6.56 mmol) was dissolved in dry dimethyl acetamide (20 ml). Sodium azide (1.7 g, 26.2 mmol) was added and the mixture heated to 90° C. for 1 hour. The reaction was cooled, diluted with ethyl acetate (100 ml), washed with water (6×50 ml), dried over magnesium sulphate and filtered. 10% Palladium on activated carbon was added and the mixture stirred under a hydrogen atmosphere for 3 hours. Filtration through celite followed by evaporation gave the title compound as a pale green solid (1.25 g, 83%). NMR (CDCl₃): 1.68 (2H, broad s), 2.67 (1H, dd), 3,2 (3H, s), 3.23 (1H, dd), 4.5-4.6 (1H, m), 4.6-4.8 (1H, m), 7.15-7.35 (4H, m); m/z 227.4 (M+H).

Method #43

(1R,2R)-2-Amino-1-[1-dimethylethoxy)carbonylamino]indan

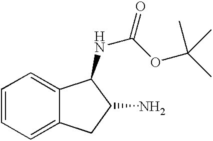

(1R,2S)-1-[(1,1-Dimethylethoxy)carbonylamino]-2-hydroxyindan (Method #41, 7.5 g, 30.1 mmol) was dissolved in dry tetrahydrofuran (90 ml) and triethylamine (6.3 ml, 45.0 mmol). Methanesulfonyl chloride (3.78 g, 33.0 mmol) dissolved in dry tetrahydrofuran (10 ml) was added and the mixture stirred at room temperature for 20 hours. The mixture was evaporated and ethyl acetate (250 ml) added. After washing with water and drying over magnesium sulphate the organic solution was evaporated to (1R,2S)-1-[(1,1-dimethylethoxy)carbonylamino]-2-methanesulphonyloxyindan as white solid (9.7 g, 98%).

NMR 1.45 (9H, s), 3.05-3.35 (2H, m), 3.18 (3H, s), 5.15-5.25 (1H, m), 5.28-5.38 (1H, m), 7.15-7.22 (4H, m), 7.45 (1H, d).

(1R,2S)-1-[(1,1-Dimethylethoxy)carbonylamino]-2-methanesulphonyloxyindan (3.5 g, 10.7 mmol) was dissolved in dry dimethyl acetamide (50 ml). Sodium azide (3.5 g, 53.9 mmol) was added and the mixture heated to 90° C. for 3 hours. The reaction was cooled, diluted with ethyl acetate (150 ml), washed with water (6×50 ml) and dried over magnesium sulphate. 10% Palladium on activated carbon was added and the mixture stirred under a hydrogen atmosphere for 4 hours. Filtration through celite followed by evaporation gave the title compound as a white solid (2.6 g, 98%). NMR: 1.45 (9H, s), 2.5 (1H, dd), 3.0 (1H, dd), 3,2-3.45 (3H, m), 4.5-4.6 (1H, m), 7.0-7.25 (5H, m).

Method #44

2,3-Dichloro-5-[N-(1-{N-[(1,1-dimethylethoxy)carbonyl]-N-methylamino}indan-2-yl)carbamoyl]-4H-thieno[3,2-b]pyrrole

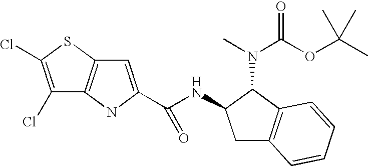

To a solution of (1R, 2S)-1-[(1,1-dimethylethoxy)carbonylamino]-2-hydroxyindan (Method #41, 7.0 g, 28.1 mmol) in dichloromethane (50 ml) was added 3,4-dihydro-2H-pyran (4.7 g, 56.2 mmol) and pyridinium toluene-4-sulphonate (100 mg). The mixture was stirred for 4 hours, diluted with ethyl acetate (200 ml), washed with water (2×50 ml), dried over magnesium sulphate and evaporated to give (1R,2S)-1-[(1,1-dimethylethoxy)carbonylamino]-2-[(tetrahydropyran-2-yl)oxy]indan as a white solid (8.9 g, 96%). NMR 1.25-1.85 (6H, m), 1.45 (9H, d), 2.85-3.1 (2H, m), 3.35-3.5 (1H, m), 3.68-3.9 (1H, m), 4.35-5.1 (3H, m), 6.8 (1H, dd), 7.1-7.3 (4H, m).

(1R,2S)-1-[(1,1-Dimethylethoxy)carbonylamino]-2-[(tetrahydropyran-2-yl)oxy]indan (4.0 g, 12.0 mmol) was dissolved in dry DMA (25 ml) at 101C and 60% sodium hydride (575 mg, 14.4 mmol) added. The mixture was stirred at room temperature for 30 minutes and then methyl iodide (2.0 g, 14.4 mmol) added after which the reaction was stirred for a further 3 hours at room temperature. The mixture was diluted with ethyl acetate (50 ml), washed with water (6×50 ml), dried over magnesium sulphate and evaporated to give (1R,2S)-1-{N-[(1,1-dimethylethoxy)]carbonyl-N-methyl-amino}-2-(tetrahydropyran-2-yl)oxyindan as an oil (4.1 g, 98%). NMR 1.4-1.9 (6H, m), 1.5 (9H, d), 2.7 (3H, dd), 2.85-3.3 (2H, m), 3.47-3.6 (1H, m), 3.72-4.0 (1H, m), 4.7-5.0 (2H, m), 5.5-5.84 (1H, m), 7.15-7.35 (4H, m); m/z 348.6 (M+H).

To a solution of (1R,2S)-1-{N-[(1,1-dimethylethoxy)carbonyl]-N-methylamino}-2-(tetrahydropyran-2-yl)oxyindan (4.0 g, 11.5 mmol) in methanol (50 ml) was added toluene-4-sulphonic acid (100 mg) and the mixture stirred at room temperature for 2 hours. Saturated aqueous sodium bicarbonate (50 ml) and water (10 ml) was added and the mixture extracted with ethyl acetate. The organic extract was washed with water, dried over magnesium sulphate and evaporated to give (1R,2S)-1-{N-[(1,1-dimethylethoxy)carbonyl]-N-methylamino}-2-hydroxyindan as an oil (3.0 g, 100%). NMR 1.45 (9H, s), 2.6 (3H, s), 2.75 (1H, dd), 3.05 (1H, dd), 4.4-4.57 (1H, m), 5.0-5.12 (1H, m), 5.34 (1H, dd), 7.03-7.33 (4H, m).

To a solution give (1R,2S)-1-{N-[(1,1-dimethylethoxy)carbonyl]-N-methylamino}-2-hydroxyindan (3.0 g, 11.4 mmol) in dry tetrahydrofuran (40 mmol) was added triethylamine (2.4 ml, 17.1 mmol) and methane sulphonyl chloride (1.44 g, 12.55 mmol). The mixture was stirred at room temperature for 1 hour, evaporated and diluted with ethyl acetate (100 ml). The organic solution was washed with saturated aqueous sodium bicarbonate and then water, dried over magnesium sulphate and evaporated to give a pale yellow syrup. The crude material was purified by silica chromatography with 4:1 iso-hexane:ethyl acetate as eluent to (1R, 2S)-1-{N-[(1,1-dimethylethoxy)carbonyl]-N-methyl-amino}-2-methanesulphonyloxyindan as a clear colourless syrup (3.1 g, 80%). NMR (CDCl₃): 1.54 (9H, s), 2.7 (3H, d), 3.0 (3H, s), 3.16-3.42 (2H, m), 5.35-5.51 (1H, m), 5.78 (1H, dd), 7.2-7.35 (4H, m); m/z 342.5 (M+H).

(1R,2S)-1-{N-[(1,1-Dimethylethoxy)carbonyl]-N-methyl-amino}-2-methanesulphonyloxyindan (3.0 g, 8.8 mmol) was dissolved in dry DMA (30 ml) and sodium azide (2.3 g, 35.2 mmol) added. The mixture was heated to 90° C. for 6 hours, cooled and diluted with ethyl acetate (100 ml). The solution was washed with water (6×50 ml), dried over magnesium sulphate and filtered. 10% Palladium on activated carbon was added and the mixture stirred under a hydrogen atmosphere for 4 hours. The mixture was filtered, evaporated and purified by silica chromatography with 10% methanol in dichloromethane to give (1R,2S)-2-amino-1-{N-[(1,1-dimethylethoxy)]carbonyl-N-methyl-amino} indan as an oil (1.2 g, 55%). NMR 1.36-1.56 (9H, m), 2.6 (3H, s), 2.7-2.87 (1H, m), 3,2-3.35 (1H, m), 4.37-4.54 (1H, m), 5.4-5.7 (1H, m), 6.93-7.1 (1H, m), 7.12-7.5 (4H, m); m/z 263.48 (M+H).

5-Carboxy-2,3-Dichloro-4H-thieno[3,2-b]pyrrole (Method #9, 472 mg, 2.0 mmol), (1R, 2S)-2-amino-1-{N-[(1,1-dimethylethoxy)]carbonyl-N-methyl-amino}indan (524 mg, 2.0 mmol), DIPEA (0.348 ml, 2.0 mmol) and HOBT (270 mg, 2.0 mmol) was stirred in dichloromethane (10 ml) at room temperature for 2 minutes. EDAC (480 mg, 2.5 mmol) was added and the mixture stirred at room temperature for 20 hours. The reaction was evaporated, ethyl acetate (50 ml) added and washed with water. The organic phase was dried over magnesium sulphate and evaporated to give the title compound as a pale brown foam (900 mg, 94%). NMR 1.2-1.45 (9H, m), 2.77 (3H, s), 2.9-3.26 (2H, m), 4.7-4.94 (1H, m), 5.5-5.8 (1H, m), 6.9-7.34 (5H, m), 8.55-8.73 (1H, m), 12.25 (1H, broad s); m/z 480.3/482.1 (M+H).

### Example 155

The following illustrate representative pharmaceutical dosage forms containing the compound of formula (I), or a pharmaceutically acceptable salt or in vivo hydrolysable ester thereof (hereafter compound X), for therapeutic or prophylactic use in humans:—

| (a): Tablet I | mg/tablet |
|---|---|
| Compound X | 100 |
| Lactose Ph.Eur | 182.75 |
| Croscarmellose sodium | 12.0 |
| Maize starch paste (5% w/v paste) | 2.25 |
| Magnesium stearate | 3.0 |

| (b): Tablet II | mg/tablet |
|---|---|
| Compound X | 50 |
| Lactose Ph.Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |

| (c): Tablet III | mg/tablet |
|---|---|
| Compound X | 1.0 |
| Lactose Ph.Eur | 93.25 |
| Croscarmellose sodium | 4.0 |
| Maize starch paste (5% w/v paste) | 0.75 |
| Magnesium stearate | 1.0 |

| (d): Capsule | mg/capsule |
|---|---|
| Compound X | 10 |
| Lactose Ph.Eur | 488.5 |
| Magnesium stearate | 1.5 |

| (e): Injection I | (50 mg/ml) |
|---|---|
| Compound X | 5.0% w/v |
| 1 M Sodium hydroxide solution | 15.0% v/v |
| 0.1 M Hydrochloric acid | (to adjust pH to 7.6) |
| Polyethylene glycol 400 | 4.5% w/v |
| Water for injection | to 100% |

| (f): Injection II | 10 mg/ml |
|---|---|
| Compound X | 1.0% w/v |
| Sodium phosphate BP | 3.6% w/v |
| 0.1 M Sodium hydroxide solution | 15.0% v/v |
| Water for injection | to 100% |

| (g): Injection III | (1 mg/ml, buffered to pH6) |
|---|---|
| Compound X | 0.1% w/v |
| Sodium phosphate BP | 2.26% w/v |
| Citric acid | 0.38% w/v |
| Polyethylene glycol 400 | 3.5% w/v |
| Water for injection | to 100% |

The above formulations may be obtained by conventional procedures well known in the pharmaceutical art. The tablets (a)-(c) may be enteric coated by conventional means, for example to provide a coating of cellulose acetate phthalate.

1. A compound of formula (I): 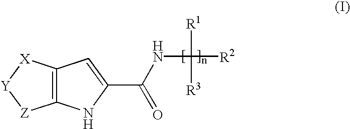
wherein:
—X-Y-Z- is selected from —S—CR⁴═CR⁵—, —CR⁴═CR⁵—S—, —O—CR⁴═CR⁵—, —CR⁴═CR⁵—O—, —N═CR⁴—S—, —S—CR⁴═N—, —NR⁶—CR⁴═CR⁵— and —CR⁴═CR⁵—NR⁶—;
wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;
R⁶ is hydrogen or C₁₋₆alkyl;
R¹ is selected from hydrogen, halo, nitro, cyano, hydroxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₄alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, C₃₋₈cycloalkylC₁₋₆alkyl, aryl, arylC₁₋₆alkyl, heterocyclic group and (heterocyclic group)C₁₋₆alkyl; wherein R¹ may be optionally substituted on carbon by one or more groups selected from P and wherein if said heterocyclic group contains an —NH— moiety that nitrogen may be optionally substituted by a group selected from R;
R² is selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₄alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, sulphamoylamino, N-(C₁₋₆alkyl)sulphamoylamino, N,N-(C₁₋₆alkyl)₂sulphamoylamino, C₁₋₆alkylsulphonylamino, C₁₋₆alkylsulphonylaminocarbonyl, C₁₋₆alkylsulphonyl-N-(C₁₋₆alkyl)amino and a group -E-F-G-H;
wherein E and G are independently selected from a direct bond, —O—, —S—, —SO—, —SO₂—, —OC(O)—, —C(O)O—, —C(O)—, —NR^{a}—, —NR^{a}C(O)—, —C(O)NR^{a}—, —SO₂NR^{a}—, —NR^{a}SO₂—, —NR^{a}C(O)NR^{b}—, —OC(O)NR^{a}—, —NR^{a}C(O)O—, —NR^{a}SO₂NR^{b}—, —SO₂NR^{a}C(O)— and —C(O)NR^{a}SO₂—; wherein R^{a} and R^{b} are independently selected from hydrogen or C₁₋₆alkyl which is optionally substituted by a group V;
F is C₁₋₆alkylene optionally substituted by one or more Q or a direct bond;
H is selected from aryl, C₃₋₈cycloalkyl and heterocyclic group; wherein H may be optionally substituted on carbon by one or more groups selected from S and wherein if said heterocyclic group contains an —NH— moiety that nitrogen may be optionally substituted by a group selected from T;
R³ is hydrogen or C₁₋₆alkyl;
n is selected from 0-4; wherein the values of R¹ may be the same or different; and wherein the values of R³ may be the same or different;
P, S and Q are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein P, S and Q may be optionally and independently substituted on carbon by one or more groups selected from V and wherein if said heterocyclic group contains an —NH— moiety that nitrogen may be optionally substituted by a group selected from U;
V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl, and 4-hydroxypiperidinocarbonyl;
R, T and U are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)carbamoyl, phenyl, benzyl, benzyloxycarbonyl, benzoyl and phenylsulphonyl wherein R, T and U may be optionally and independently substituted on carbon by one or more groups selected from V;
or a pharmaceutically acceptable salt or an in vivo hydrolysable ester thereof;
with the provisos:
i) when —X-Y-Z- is —S—CH═CH—, R²—(CR¹R³)ₙ— cannot be amino, 1-phenyl-5-methyl-1H-1,5-benzodiazepine-2,4 (3H,5H)dion-3-yl, 1-methyl-5-phenyl-2-oxo-2,3-dihydro-1H-benzo(E)(1,4)diazepin-3-yl, 2-(4-phenyl-1,2,5,6-tetrahydropyrid-1-yl)ethyl, 3-(4-phenyl-1,2,5,6-tetrahydropyrid-1-yl)propyl, 2-(4-phenylpiperazin-1-yl)ethyl, 2-(N-methylamino)ethyl, 2-morpholinoethyl or 2-(N-methyl-N-benzylamino)ethyl;
ii) when —X-Y-Z- is —CH═CH—S—, R²—(CR¹R³)ₙ— cannot be amino or 1-methyl-5-phenyl-2-oxo-2,3-dihydro-1H-benzo(E)(1,4)diazepin-3-yl;
iii) when —X-Y-Z- is —CH═C(SO₂NH₂)—S—, R²—(CR¹R³)ₙ— cannot be methyl or isobutyl; and
iv) when —X-Y-Z- is as initially defined, n is 1, R¹ is arylmethyl, substituted arylmethyl, (heterocyclic group)methyl and substituted (heterocyclic group)methyl and R³ is hydrogen then R² is not a group —C(═O)-A or a group —CH(OH)—C(═O)-A in which A is NR^{d}R^{d}, —NR^{a}CH₂CH₂OR^{a}, or 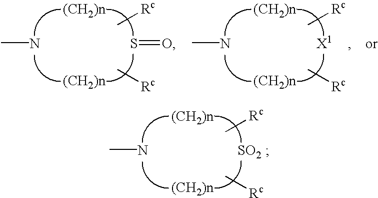
each R^{a} and R^{b} is independently hydrogen or C₁-C₈alkyl;
each R^{d} is independently hydrogen, C₁-C₈alkyl, C₁-C₈alkoxy, aryl, substituted aryl, heteroaryl, or substituted heteroaryl;
each R^{c} is independently hydrogen, —C(═O)OR^{a}, —OR^{a}, SR^{a}, or N^{a}R^{a}; and each n is independently 1-3, and
X¹ is NR^{a}, —CH₂—, O or S.

2. A compound of formula (I) according to claim 1 wherein:
—X-Y-Z- is selected from —S—CR⁴═CR⁵— or —CR⁴═CR⁵—S—;
wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;
n is 0;
R² is a group -E-F-G-H;
wherein E, F and G are each a direct bond;
H is a C₃₋₁₂cycloalkyl which is optionally fused to a benz ring wherein H may be optionally substituted on carbon by one or more groups S which are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic groups; wherein S may be optionally substituted on carbon by one or more groups selected from V;
V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl, and 4-hydroxypiperidinocarbonyl;
or a pharmaceutically acceptable salt thereof.

3. A compound of formula (I) as claimed in claim 1 wherein:
—X-Y-Z- is selected from —S—CR⁴═CR⁵— or —CR⁴═CR⁵—S—;
wherein R⁴ and R⁵ are independently selected from hydrogen, halo, nitro, cyano, hydroxy, fluoromethyl, difluoromethyl, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino and C₁₋₆alkylsulphonyl-N-(C₁₋₆alkyl)amino;
n is 0;
R² is a group -E-F-G-H;
wherein E, F and G are each a direct bond; and
H is a cyclic amide of formula 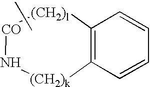
in which k is 0, 1, 2 or 3 and l is 0, 1, 2 or 3 such that the sum of k and l is 2 or 3 and wherein one of the carbon atoms governed by k or l may be replaced by sulphur and wherein H is optionally substituted on carbon by one or more groups selected from S and may be independently optionally substituted on nitrogen by a group selected from T;
S is selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein S may be optionally and independently substituted on carbon by one or more groups selected from V and wherein if said heterocyclic group contains an —NH— moiety that nitrogen may be optionally substituted by a group selected from U;
T and U are independently selected from C₁₋₄alkyl, C₁₋₄alkanoyl, C₁₋₄alkylsulphonyl, C₁₋₄alkoxycarbonyl, carbamoyl, N-(C₁₋₄alkyl)carbamoyl, N,N-(C₁₋₄alkyl)carbamoyl, phenyl, benzyl, benzyloxycarbonyl, benzoyl and phenylsulphonyl wherein R, T and U may be optionally and independently substituted on carbon by one or more groups selected from V;
V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl and 4-hydroxypiperidinocarbonyl;
or a pharmaceutically acceptable salt or an in vivo hydrolysable ester thereof.

4. A compound of formula (I) as claimed in claim 1 wherein:
—X-Y-Z- is selected from —S—CR⁴═CR⁵— or —CR⁴═CR⁵—S—;
wherein R⁴ and R⁵ are independently selected from hydrogen, halo or C₁₋₆alkyl.
n is 1;
R¹ is hydrogen or arylC₁₋₆alkyl;
R² is selected from a group -E-F-G-H;
wherein E, F and G are each a direct bond;
H is an unsaturated five membered heterocyclic group containing at least one nitrogen atom and one or two ring atoms selected from oxygen and sulphur and wherein H may be optionally substituted on carbon by one or more groups S which are independently selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O), wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl and aryl groups;
R³ is hydrogen or C₁₋₆alkyl;
or a pharmaceutically acceptable salt thereof.

5. A compound of formula (I) as claimed in claim 1 wherein:
—X-Y-Z- is selected from —S—CR⁴═CR⁵— or —CR⁴═CR⁵—S—;
wherein R⁴ and R⁵ are independently selected from hydrogen, halo or C₁₋₆alkyl.
n is 0;
R² is a group -E-F-G-H;
wherein E is a direct bond;
F is methylene;
wherein G is —C(O)NR^{a}—, wherein R^{a} is selected from hydrogen or C₁₋₆alkyl which is optionally substituted by a group V;
H is aryl which may be optionally substituted on carbon by one or more groups selected from S;
S is selected from halo, nitro, cyano, hydroxy, trifluoromethyl, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, N-(C₁₋₆alkyl)-N-(C₁₋₆alkoxy)carbamoyl, C₁₋₆alkylS(O)ₐ wherein a is 0 to 2, C₁₋₆alkoxycarbonyl, C₁₋₆alkoxycarbonylamino, N-(C₁₋₆alkyl)sulphamoyl, N,N-(C₁₋₆alkyl)₂sulphamoyl, C₁₋₆alkylsulphonylamino, C₁₋₆alkylsulphonyl-N-(C₁₋₆alkyl)amino, C₃₋₈cycloalkyl, aryl and heterocyclic group; wherein S may be optionally and independently substituted on carbon by one or more groups selected from V;
V is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, morpholino, morpholinocarbonyl, N-benzylcarbamoyl, and 4-hydroxypiperidinocarbonyl;
or a pharmaceutically acceptable salt thereof.

6. A compound selected from
2,3-dichloro-5-[N-(2-phenoxyethyl)carbamoyl]-4H-thieno[3,2-b]pyrrole;
2,3-dichloro-5-{N-[2-(2-thienyl)ethyl]carbamoyl}-4H-thieno[3,2-b]pyrrole;
2,3-dichloro-5-{N-[2-(2-methoxyphenyl)ethyl]carbamoyl}-4H-thieno[3,2-b]pyrrole;
2,3-dichloro-5-[N-(2-phenyl-1-cyclopropyl)carbamoyl]-4H-thieno[3,2-b]pyrrole;
2,3-dichloro-5-{N-[2-(4-fluorophenyl)ethyl]carbamoyl}-4H-thieno[3,2-b]pyrrole;
2,3-dichloro-5-[N-(N-phenylcarbamoylmethyl)carbamoyl]-4H-thieno[3,2-b]pyrrole;
2,3-dichloro-5-(N-{2-[(2-pyridyl)amino]ethyl}carbamoyl)-4H-thieno[3,2-b]pyrrole;
2,3-dichloro-5-{N-[2-(N-methylmethanesulphonamido)-1-(thiazol-2-yl)ethyl]carbamoyl}-4H-thieno[3,2-b]pyrrole;
2,3-dichloro-5-{N-[2-(thiomorpholino)ethyl]carbamoyl}-4H-thieno[3,2-b]pyrrole;
5-[N-(benzoylmethyl)carbamoyl]-2,3-dichloro-4H-thieno[3,2-b]pyrrole;
3-chloro-5-[N-(N-phenylcarbamoylmethyl)carbamoyl]-4H-thieno[3,2-b]pyrrole;
3-chloro-5-{N-[2-(thiomorpholino)ethyl]carbamoyl}-4H-thieno[3,2-b]pyrrole;
3-chloro-5-{N-[2-(N-methylmethanesulphonamido)-1-(thiazol-2-yl)ethyl]carbamoyl}-4H-thieno[3,2-b]pyrrole;
3-chloro-5-[N-(benzoylmethyl)carbamoyl]-4H-thieno[3,2-b]pyrrole;
3-chloro-5-{N-[2-(2-methoxyphenyl)ethyl]carbamoyl}-4H-thieno[3,2-b]pyrrole;
3-chloro-5-{N-[2-(2-thienyl)ethyl]carbamoyl}-4H-thieno[3,2-b]pyrrole;
3-chloro-5-[N-(2-phenyl-1-cyclopropyl)carbamoyl]-4H-thieno[3,2-b]pyrrole;
3-chloro-5-{N-[2-(4-fluorophenyl)ethyl]carbamoyl}-4H-thieno[3,2-b]pyrrole;
3-chloro-5-[N-(2-phenoxyethyl)carbamoyl]-4H-thieno[3,2-b]pyrrole;
3-chloro-5-{(N-[2-(1-phenylmethanesulphonamido)ethyl]carbamoyl}-4H-thieno[3,2-b]pyrrole;
3-chloro-5-[N-(4-oxo-2,3,4,5-tetrahydrobenz[1,5]thiazepin-3-yl)carbamoyl]-4H-thieno[3,2-b]pyrrole;
2-chloro-5-[N-(benzoylmethyl)carbamioyl]-4H-thieno[3,2-b]pyrrole;
2-chloro-5-[N-(2-phenyl-1-cyclopropyl)carbamoyl]-4H-thieno[3,2-b]pyrrole;
2-chloro-5-[N-(N-phenylcarbamoylmethyl)carbamoyl]-4H-thieno[3,2-b]pyrrole;
2-chloro-5-(N-{2-[(2-pyridyl)amino]ethyl}carbamoyl)-4H-thieno[3,2-b]pyrrole;
2-chloro-5-{N-[2-(2-methoxyphenyl)ethyl]carbamoyl}-4H-thieno[3,2-b]pyrrole;
2-chloro-5-[N-(2-phenoxyethyl)carbamoyl]-4H-thieno[3,2-b]pyrrole;
2-chloro-5-{N-[2-(2-thienyl)ethyl]carbamoyl}-4H-thieno[3,2-b]pyrrole;
2-chloro-5-{N-[2-(4-fluorophenyl)ethyl]carbamoyl}-4H-thieno[3,2-b]pyrrole;
2-chloro-5-{N-[2-(N-methylmethanesulphonamido)-1-(thiazol-2-yl)ethyl]carbamoyl}-4H-thieno[3,2-b]pyrrole;
2-chloro-5-{N-[2-(thiomorpholino)ethyl]carbamoyl}-4H-thieno[3,2-b]pyrrole;
2,3-dichloro-5-[N-(2,3-dimethyl-5-oxo-1-phenyl-2,5-dihydro-1H-pyrazol-4-yl)carbamoyl]-4H-thieno[3,2-b]pyrrole;
2,3-dichloro-5-[N-(4-sulphamoylphenylmethyl)carbamoyl]-4H-thieno[3,2-b]pyrrole;
2,3-dichloro-5-[N-(2-hydroxy-1-phenethyl)carbamoyl]-4H-thieno[3,2-b]pyrrole;
2,3-dichloro-5-{N-(2-[(3-trifluoromethylpyrid-2-yl)amino]ethyl)carbamoyl}-4H-thieno[3,2-b]pyrrole;
2,3-dichloro-5-{N-[3-(5-tetrazolyl)propyl]carbamoyl}-4H-thieno[3,2-b]pyrrole;
2,3-dichloro-5-[N-(5-oxo-3-phenyl-4,5-dihydroisoxazol-4-yl)carbamoyl]-4H-thieno[3,2-b]pyrrole;
2,3-dichloro-5-[N-(5-hydroxy-2-oxo-2,3,4,5-tetrahydro-1H-benz[b]azepin-4-yl)carbamoyl]-4H-thieno[3,2-b]pyrrole;
2-chloro-5-{N-[3-(benzyloxycarbonylamino)propyl]carbamoyl}-4H-thieno[3,2-b]pyrrole;
2,3-dichloro-5-{N-[(4-dimethylaminophenyl)methyl]carbanoyl}-4H-thieno[3,2-b]pyrrole;
5-[N-(1-benzyl-2-hydroxyethyl)carbamoyl]-2,3-dichloro-4H-thieno[3,2-b]pyrrole;
2,3-dichloro-5-{N-[2-(phenylamino)ethyl]carbamoyl}-4H-thieno[3,2-b]pyrrole;
2,3-dichloro-5-[N-(β-(R)-hydroxy-α-methylphenethyl)carbamoyl]-4H-thieno[3,2-b]pyrrole;
2,3-dichloro-5-[N-(β-hydroxyphenethyl)carbamoyl]-4H-thieno[3,2-b]pyrrole;
2,3-dichloro-5-{N-[2-(4-hydroxyphenyl)ethyl]carbamoyl}4H-thieno[3,2-b]pyrrole;
2,3-dichloro-5-{N-[(benzimidazol-2-yl)methyl]carbamoyl}-4H-thieno[3,2-b]pyrrole;
2,3-dichloro-5-{N-[2-(4-chlorophenyl)-2-hydroxy-1-(methoxycarbonyl)ethyl]carbamoyl}-4H-thieno[3,2-b]pyrrole;
2,3-dichloro-5-{N-(imidazo[1,2-a]pyrid-2-yl)carbamoyl}-4H-thieno[3,2-b]pyrrole;
5-{N-[(benzthiazol-2-yl)methyl]carbamoyl}-2,3-dichloro-4H-thieno[3,2-b]pyrrole;
2,3-dichloro-5-{N-[(6-trifluoromethylpyrid-3-yl)methyl]carbamoyl)-4H-thieno[3,2-b]pyrrole;
2,3-dichloro-5-{N-(2-[(2-pyridazinyl)methyl]carbamoyl}-4H-thieno[3,2-b]pyrrole;
2,3-dichloro-5-{N-[N-(2-hydroxy-3-phenoxypropyl)carbamoylmethyl] carbamoyl}-4H-thieno[3,2-b]pyrrole;
2,3-dichloro-5-{N-[N-(3-methylisothiazol-5-yl)carbamoylmethyl]carbamoyl}-4H-thieno[3,2-b]pyrrole;
2,3-dichloro-5-{N-[2-(pyridazin-3-yloxy)ethyl]carbamoyl}-4H-thieno[3,2-b]pyrrole;
2-chloro-5-(N-{2-[(3-trifluoromethylpyrid-2-yl)amino]ethyl}carbamoyl)-4H-thieno[3,2-b]pyrrole;
2,3-dichloro-5-{N-[2-(4-sulphamoylphenyl)ethyl]carbamoyl}-4H-thieno[3,2-b]pyrrole;
2,3-dichloro-5-{N-[2-(2-pyridyl)ethyl]carbamoyl}-4H-thieno[3,2-b]pyrrole;
2,3-dichloro-5-{N-(2-[1-hydroxymethyl-2-(4-imidazolyl)ethyl]carbamoyl}-4H-thieno[3,2-b]pyrrole;
2,3-dichloro-5-{N-(2-[(3-quinolyl)methyl]carbamoyl}-4H-thieno[3,2-b]pyrrole;
5-{N-[3-(4-acetamidophenoxy)-2-hydroxypropyl]carbamoyl}-2,3-dichloro-4H-thieno[3,2-b]pyrrole;
2,3-dichloro-5-{N-[3-(N-methylsulphonylcarbamoyl)propyl]carbamoyl}-4H-thieno[3,2-b]pyrrole;
2,3-dichloro-5-[N-(2-{[2-(guanidino)thiazol-4-yl]methylthio}ethyl)carbamoyl]-4H-thieno[3,2-b]pyrrole;
2,3-dichloro-5-{N-[2-(2,4-dioxoimidazolidin-1-yl)ethyl]carbamoyl}-4H-thieno[3,2-b]pyrrole;
5-N-[2-benzylthio-1-(hydroxymethyl)ethyl]carbamoyl}-2,3-dichloro-4H-thieno[3,2-b]pyrrole;
2,3-dichloro-5-{N-[2-(dimethyaminosulphonylamino)ethyl]carbamoyl}-4H-thieno[3,2-b]pyrrole;
2,3-dichloro-5-{N-[(6-methoxypyrid-3-yl)methyl]carbamoyl}-4H-thieno[3,2-b]pyrrole;
(S)-2,3-dichloro-5-{N-[(2-oxo-3-phenyl-2,3,4,5-tetrahydrooxazol-5-yl)methyl]carbamoyl}-4H-thieno[3,2-b]pyrrole;
2,3-dichloro-5-(N-{2-[3-(carbamoylmethyl)phenoxy]ethyl}carbamoyl)-4H-thieno[3,2-b]pyrrole;
5-(N-{[6-(benzo[1,3]dioxol-5-yl)-4-methylmorpholin-2-yl]methyl}carbamoyl)-2,3-dichloro-4H-thieno[3,2-b]pyrrole;
5-(N-benzylcarbamoyl)-2,3-dichloro-4H-thieno[3,2-b]pyrrole;
2,3-dichloro-5-(N-phenethylcarbamoyl)-4H-thieno[3,2-b]pyrrole;
2,3-dichloro-5-[N-(3-phenylpropyl)carbamoyl]-4H-thieno[3,2-b]pyrrole;
2,3-dichloro-5-[N-[2-(2-hydroxyphenyl)ethyl]carbamoyl}-4H-thieno[3,2-b]pyrrole;
2,3-dichloro-5-[N-(α,α-dimethylphenethyl)carbamoyl]-4H-thieno[3,2-b]pyrrole;
2,3-dichloro-5-[N-(1-phenylcyclobutyl)methyl)carbamoyl]-4H-thieno[3,2-b]pyrrole;
2,3-dichloro-5-[N-(β-methylphenethyl)carbamoyl]-4H-thieno[3,2-b]pyrrole;
2,3-dichloro-5-[N-(1,2,3,4-tetrahydronaphth-2-yl)carbamoyl]-4H-thieno[3,2-b]pyrrole;
5-[N-(N-benzylcarbamoylmethyl)carbamoyl]-2,3-dichloro-4H-thieno[3,2-b]pyrrole;
5-[N-(N-benzyl-N-methylcarbamoylmethyl)carbamoyl]-2,3-dichloro-4H-thieno[3,2-b]pyrrole;
2,3-dichloro-5-[N-(N-methyl-N-phenylcarbamoylmethyl)carbamoyl]-4H-thieno[3,2-b]pyrrole;
2,3-dichloro-5-{N-[N-(2-cyanoethyl)-N-phenylcarbamoylmethyl]carbamoyl}-4H-thieno[3,2-b]pyrrole;
2,3-dichloro-5-{N-[N-(4-methoxyphenyl)carbamoylmethyl]carbamoyl}-4H-thieno[3,2-b]pyrrole;
2,3-dichloro-5-{N-[N-(4-fluorophenyl)carbamoylmethyl]carbamoyl}-4H-thieno[3,2-b]pyrrole;
2,3-dichloro-5-{N-[N-(4-nitrophenyl)carbamoylmethyl]carbamoyl}-4H-thieno[3,2-b]pyrrole;
2,3-dichloro-5-{N-[N-(2,6-dimethylphenyl)carbamoylmethyl]carbamoyl}-4H-thieno[3,2-b]pyrrole;
2,3-dichloro-5-{N-[N-methyl-N-(4-methylphenyl)carbamoylmethyl]carbamoyl}-4H-thieno[3,2-b]pyrrole;
2,3-dichloro-5-{N-[N-methyl-N-(3-methylphenyl)carbamoylmethyl]carbamoyl}-4H-thieno[3,2-b]pyrrole;
2,3-dichloro-5-{N-[N-(3-chlorophenyl) N-methylcarbamoylmethyl]carbamoyl}-4H-thieno[3,2-b]pyrrole;
2,3-dichloro-5-{N-[N-(2-hydroxyethyl)-N-phenylcarbamoylmethyl]carbamoyl}-4H-thieno[3,2-b]pyrrole;
2,3-dichloro-5-{N-[N-(1,1-dimethyl-2-hydroxyethyl)carbamoylmethyl]carbamoyl}-4H-thieno[3,2-b]pyrrole;
2,3-dichloro-5-{N-[N-(2-hydroxyethyl)-N-methylcarbamoylmethyl]carbamoyl}-4H-thieno[3,2-b]pyrrole;
2,3-dichloro-5-{N-[N-(2-hydroxyethyl)carbamoylmethyl]carbamoyl}-4H-thieno[3,2-b]pyrrole;
2,3-dichloro-5-{N-[N-(3-hydroxypropyl)carbamoylmethyl]carbamoyl}-4H-thieno[3,2-b]pyrrole;
2,3-dichloro-5-{N-[N-(4-hydroxybutyl)carbamoylmethyl]carbamoyl}-4H-thieno[3,2-b]pyrrole;
2,3-dichloro-5-(N-{N-[bis(hydroxymethyl)methyl]carbamoylmethyl}carbamoyl)-4H-thieno[3,2-b]pyrrole;
2,3-dichloro-5-{N-[N-(2,3-dihydroxypropyl)carbamoylmethyl]carbamoyl}-4H-thieno[3,2-b]pyrrole;
2,3-dichloro-5-{N-[N-(4-hydroxymethylphenyl)carbamoylmethyl]carbamoyl}-4H-thieno[3,2-b]pyrrole;
2,3-dichloro-5-{N-[N-(5-isoquinolyl)carbamoylmethyl]carbamoyl}-4H-thieno[3,2-b]pyrrole;
2,3-dichloro-5-{N-[N-(3-hydroxymethy)lphenyl]carbamoylmethyl]carbamoyl}-4H-thieno[3,2-b]pyrrole;
2,3-dichloro-5-(N-{N-[4-(2-hydroxyethyl)phenyl]carbamoylmethyl}carbamoyl)-4H-thieno[3,2-b]pyrrole;
2,3-dichloro-5-{N-[N-(2,4-difluorophenyl)-N-methyl-carbamoylmethyl]carbamoyl}-4H-thieno[3,2-b]pyrrole;
2,3-dichloro-5-{N-[(1,2,3,4-tetrahydro-1-quinolyl)carbonylmethyl]carbamoyl}-4H-thieno[3,2-b]pyrrole;
2,3-dichloro-5-{N-[N-(2-cyanoethyl)-N-methylcarbamoylmethyl]carbamoyl}-4H-thieno[3,2-b]pyrrole;
2,3-dichloro-5-{N-[N-(4-hydroxypiperidino)carbamoylmethyl]carbamoyl}-4H-thieno[3,2-b]pyrrole;
2,3-dichloro-5-[N-(N-cyclopentylcarbamoylmethyl)carbamoyl]-4H-thieno[3,2-b]pyrrole;
2,3-dichloro-5-[N-(N-isopropylcarbamoylmethyl)carbalioyl]-4H-thieno[3,2-b]pyrrole;
2,3-dichloro-5-[N-(N-isopropyl-N-methylcarbamoylmethyl)carbamoyl]-4H-thieno[3,2-b]pyrrole;
2,3-dichloro-5-[N-(thiomorpholinocarbonylmethyl)carbamoyl]-4H-thieno[3,2-b]pyrrole;
2,3-dichloro-5-[N-(morpholinocarbonylmethyl)carbamoyl]-4H-thieno[3,2-b]pyrrole;
2,3-dichloro-5-{N-[(1,1-dioxothiomorpholino)carbonylmethyl]carbamoyl}-4H-thieno[3,2b]-pyrrole;
2,3-dichloro-5-{N-[(1-oxothiomorpholino)carbonylmethyl]carbamoyl}-4H-thieno[3,2-b]pyrrole;
2-chloro-5-[N-(2-indanyl)carbamoyl]-6H-thieno[2,3-b]pyrrole;
5-[N-(benz [1,2]oxazol-3-ylmethyl)carbamoyl]-2,3-dichloro-4H-thieno[3,2-b]pyrrole;
2,3-dichloro-5-(N-{2-[2-(hydroxymethyl)phenyl]ethyl}carbamoyl)-4H-thieno[3,2-b]pyrrole;
2,3-dichloro-5-[N-(4-phenylisoxazol-3-ylmethyl)carbamoyl]-4H-thieno[3,2-b]pyrrole;
2,3-dichloro-5-(N-{2-[2-(2-morpholinoethoxy)phenyl]ethyl}carbamoyl)-4H-thieno[3,2-b]pyrrole;
2,3-dichloro-5-(N-{2-[2-(methoxycarbonylmethoxy)phenyl]ethyl}carbamoyl)-4H-thieno[3,2-b]pyrrole;
5-(N-{2-[2-(carboxymethoxy)phenyl]ethyl}carbamoyl)-2,3-dichloro-4H-thieno[3,2-b]pyrrole;
2,3-dichloro-5-{N-[2-(3-methoxyphenyl)ethyl]carbamoyl}-4H-thieno[3,2-b]pyrrole;
2,3-dichloro-5-[N-(2-oxo-1,2,3,4-tetrahydroquinol-3-yl)carbamoyl]-4H-thieno[3,2-b]pyrrole;
2,3-dichloro-5-(N-{2-[2-(2-methoxyethoxy)phenyl]ethyl}carbamoyl)-4H-thieno[3,2-b]pyrrole;
5-(N-{2-[2-(carbamoylmethoxy)phenyl]ethyl}carbamoyl)-2,3-dichloro-4H-thieno[3,2-b]pyrrole;
2,3-dichloro-5-(N-{2-[2-(N-methylcarbamoylmethoxy)phenyl]ethyl}carbamoyl)-4H-thieno[3,2-b]pyrrole;
2,3-dichloro-5-(N-{2-[2-(N,N-dimethylcarbamoylmethoxy)phenyl]ethyl}carbamoyl)-4H-thieno[3,2-b]pyrrole;
2,3-dichloro-5-(N-{2-[2-(morpholinocarbonylmethoxy)phenyl]ethyl}carbamoyl)-4H-thieno[3,2-b]pyrrole;
5-(N-{2-[2-(N-benzylcarbamoylmethoxy)phenyl]ethyl}carbamoyl)-2,3-dichloro-4H-thieno[3,2-b]pyrrole;
2,3-dichloro-5-(N-{2-[[2-(4-hydroxypiperidinocarbonylmethoxy)phenyl]ethyl}carbamoyl)-4H-thieno[3,2-b]pyrrole;
(S)-2-chloro-5-{N-[α-(5-ethoxycarbonyl-1,3,4-oxadiazol-2-yl)phenethyl]carbamoyl}-6H-thieno[2,3-b]pyrrole;
(S)-2-chloro-5-{N-[α-(4-methoxycarbonyloxazol-5-yl)phenethyl]carbamoyl}-6H-thieno[2,3-b]pyrrole;
2-chloro-5-{N-[α-(3-pyridyl)phenethyl]carbamoyl}-6H-thieno[2,3-b]pyrrole;
2,3-dichloro-5-{N-[α-(3-pyridyl)phenethyl)]carbamoyl}-4H-thieno[3,2-b]pyrrole;
(S)-2-chloro-5-{N-[a-(3-phenyl-1,2,4-oxadiazol-5-yl)phenethyl]carbamoyl}-6H-thieno[2,3-b]pyrrole;
2,3-dichloro-5-[N-(1-hydroxyindan-2-yl)carbamoyl]-4H-thieno[3,2-b]pyrrole;
2,3-dichloro-5-[N-((1S, 2S)-1-hydroxyindan-2-yl)carbamoyl]-4H-thieno[3,2-b]pyrrole;
2,3-dichloro-5-[N-((1R, 2R)-1-hydroxyindan-2-yl)carbamoyl]-4H-thieno[3,2-b]pyrrole;
2-chloro-5-[N-(1-hydroxyindan-2-yl)carbamoyl]-6H-thieno[2,3-b]pyrrole;
2,3-dichloro-5-[N-(1-hydroxy-1,2,3,4-tetrahydronaphth-2-yl)carbamoyl]-4H-thieno[3,2-b]pyrrole;
2,3-dichloro-5-[N-(6-fluoro-1-hydroxyindan-2-yl)carbamoyl]-4H-thieno[3,2-b]pyrrole;
2,3-dichloro-5-[N-(7-methoxy-1-oxo-1,2,3,4-tetrahydronaphth-2-yl)carbamoyl]-4H-thieno[3,2-b]pyrrole;
2,3-dichloro-5-[N-(2-indanyl)carbamoyl]-4H-thieno[3,2-b]pyrrole;
2,3-dichloro-5-[N-(3-methylisoxazol-5-yl)methyl]carbamoyl]-4H-thieno[3,2-b]pyrrole;
2,3-dichloro-5-[N-(4-hydroxy-1,1-dioxotetrahydrothiophen-3-yl)carbamoyl]-4H-thieno[3,2-b]pyrrole;
2,3-dichloro-5-(N-{N-methyl-N-[(1-oxo-1,2,3,4-tetrahydronaphth-2-yl)methyl]carbamoylmethyl}carbamoyl)-4H-thieno[3,2-b]pyrrole;
2-chloro-5-[N-(2-oxo-1,2,3,4-tetrahydroquinol-3-yl)carbamoyl]-6H-thieno[2,3-b]pyrrole;
2-chloro-5-[N-(1,2,3,4-tetrahydroquinol-3-yl)carbamoyl]-6H-thieno[2,3-b]pyrrole;
2-chloro-5-[N-(1-methyl-2-oxo-1,2,3,4-tetrahydroquinol-3-yl)carbamoyl]-6H-thieno[2,3-b]pyrrole;
2-chloro-5-[N-(3-oxo-2,3,4,5-tetrahydro-1H-benz [2]azepin-4-yl)carbamoyl]-6H-thieno[2,3-b]pyrrole;
2,3-dichloro-5-[N-(1-methoxyindan-2-yl)carbamoyl]-4H-thieno[3,2-b]pyrrole;
2,3-dichloro-5-(N-{1-[N-(1,1-dimethylethoxy)carbonylamino]indan-2-yl}carbamoyl)-4H-thieno[3,2-b]pyrrole;
5-[N-(1-aminoindan-2-yl)carbamoyl]-2,3-dichloro-4H-thieno[3,2-b]pyrrole;
5-[N-(1-acetamidoindan-2-yl)carbamoyl]-2,3-dichloro-4H-thieno[3,2-b]pyrrole;
2,3-dichloro-5-{N-[1-(methanesulphonamido)indan-2-yl]carbamoyl}-4H-thieno[3,2-b]pyrrole;
2,3-dichloro-5-{N-[1-(methylamino)indan-2-yl]carbamoyl}-4H-thieno[3,2-b]pyrrole; and
2,3-dichloro-5-{N-[1-(N-methylacetamido)indan-2-yl]carbamoyl}-4H-thieno[3,2-b]pyrrole
or a pharmaceutically acceptable salt thereof.

7. A pharmaceutical composition which comprises a compound of the formula (I) as claimed in any one of claims 1-6, or a pharmaceutically acceptable salt or in vivo hydrolysable ester thereof, as defined hereinbefore in association with a pharmaceutically-acceptable diluent or carrier.

8. The use of a compound of the formula as claimed in any one of claims 1 to 6 or a pharmaceutically acceptable salt or in vivo hydrolysable ester thereof as a medicament.

9. The use of a compound of the formula as claimed in any one of claims 1 to 6 or a pharmaceutically acceptable salt or in vivo hydrolysable ester thereof, as defined hereinbefore in the manufacture of a medicament for use in the production of a glycogen phosphorylase inhibitory effect in a warm-blooded animal such as man.
